SEQUENCE LISTING (8 sequences)

SEQ ID NO: 1 - DNA, 20 bp, Artificial
  Sense primer B19-21
tggcagaccagtttcgtgaa

SEQ ID NO: 2 - DNA, 20 bp, Artificial
  Antisense primer B19-22
ccggcaaacttccttgaaaa

SEQ ID NO: 3 - DNA, 18 bp, Artificial
  Probe B19-V23, with a VIC tag in 5′
cagctgcccctgtggccc

SEQ ID NO: 4 - DNA, 19 bp, Artificial
  Sense primer actin-S
ggcacccagcacaatgaag

SEQ ID NO: 5 - DNA, 20 bp, Artificial
  Antisense primer actin-AS
gccgatccacacggagtact

SEQ ID NO: 6 - DNA, 26 bp, Artificial
  Probe actin-FAM, with a FAM tag in 5′
tcaagatcattgctcctcctgagcgc

SEQ ID NO: 7 - DNA, 2151 bp, Artificial
  Fucci Sensor nucleic acid sequence
atggtcagcaagggggaagaagacaatatggctatcatcaaagagtttatgcggttcaaggtgcacatggaaggaagcgtcaatgggcacgagttcgagatcgaaggagagggagagggcagaccctacgagggcacccagacagccaagctgaaggtgaccaagggaggaccactgcctttcgcctgggacatcctgagcccacagtttatgtacggctccaaggcctatgtgaagcaccccgccgacatccctgattatctgaagctgtccttccccgagggctttaagtgggagagagtgatgaattttgaggatggcggcgtggtgaccgtgacacaggacagctccctgcaggatggcgagttcatctacaaggtgaagctgaggggcaccaactttccatctgacggccccgtgatgcagaagaagacaatgggctgggaggcctctagcgagcgcatgtatcctgaggatggcgccctgaagggcgagatcaagcagcggctgaagctgaaggacggcggccactacgatgccgaggtgaagaccacatataaggccaagaagcctgtgcagctgccaggcgcctacaacgtgaatatcaagctggacatcaccagccacaacgaggattacacaatcgtggagcagtatgagagggcagagggccgccactccaccggaggcatggacgagctgtacaagggctatccatctcactggcggcccctggagccaagcccagcccggcctgccctgagagcaccagcaagcgccacaagcggctccagaaagagggccagaccccctgcagcacctggaagggatcaggcccgcccaccagcacggagaaggctgaggctgtccgtggacgaggtgtcctctccatctaccccagaggcacctgatatcccagcatgccctagcccaggccagaagatcaagaagtccacaccagcagcaggacagcctccacacctgacctccgcccaggaccaggatacaatccagctggagggaaggggctctctgctgacctgtggcgacgtggaggagaatcccggccctatggtgtccaagggcgaggagctgttcacaggcgtggtgcccatcctggtggagctggacggcgatgtgaacggccacaagttttctgtgagcggagagggagagggcgatgcaacctacggcaagctgacactgaagctgatctgcaccacaggcaagctgccagtgccctggcctaccctggtgaccacactgggctacggcctgcagtgtttcgcccggtatcctgaccacatgaagcagcacgatttctttaagagcgccatgccagagggctacgtgcaggagcggaccatcttctttaaggacgatggcaattataagaccagagccgaggtgaagttcgagggcgacacactggtgaacaggatcgagctgaagggcatcgactttaaggaggatggcaatatcctgggccacaagctggagtacaactataattcccacaacgtgtacatcaccgccgataagcagaagaacggcatcaaggccaatttcaagatcagacacaacatcgaggacggaggagtgcagctggcagatcactatcagcagaacacaccaatcggcgacggacccgtgctgctgcctgataatcactacctgtcctatcagtctaagctgagcaaggaccctaatgagaagcgcgatcacatggtgctgctggagtttgtgaccgcagcaggaatcacactgggaatggatgaactctacaaaggatatcccagccactggagacctcttgagatgaacccaagcatgaagcagaagcaggaggagatcaaggagaacatcaagaatagctccgtgccacgccggaccctgaagatgatccagccatccgcctctggcagcctggtgggaagggagaacgagctgtccgccggcctgtctaagcggaagcacagaaatgaccacctgacctctaccacatctagccccggcgtgatcgtgcctgagtcctctgagaacaagaatctgggcggcgtgacacaggagagctttgatctgatgatcaaggaaaacccatcaagccagtattggaaagaggtcgctgagaaaagaagaaaagccctgtaa

SEQ ID NO: 8 - protein, 716 aa, Artificial
  Fucci Sensor Protein sequence
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGYPSHWRPLEPSPARPALRAPASATSGSRKRARPPAAPGRDQARPPARRRLRLSVDEVSSPSTPEAPDIPACPSPGQKIKKSTPAAGQPPHLTSAQDQDTIQLEGRGSLLTCGDVEENPGPMVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSKLSKDPNEKRDHMVLLEFVTAAGITLGMDELYKGYPSHWRPLEMNPSMKQKQEEIKENIKNSSVPRRTLKMIQPSASGSLVGRENELSAGLSKRKHRNDHLTSTTSSPGVIVPESSENKNLGGVTQESFDLMIKENPSSQYWKEVAEKRRKAL

(19) United States
(12) Patent Application Publication
(10) Pub. No.: US 2024/0101958 A1
(43) Pub. Date: Mar. 28, 2024

(54) NOVEL HUMAN ERYTHROID PROGENITOR CELL LINE HIGHLY PERMISSIVE TO B19 INFECTION AND USES THEREOF

(71) Applicants: COMMISSARIAT A L'ENERGIE ATOMIQUE ET AUX ENERGIES ALTERNATIVES, PARIS (FR); LABORATOIRE FRANCAIS DU FRACTIONNEMENT ET DES BIOTECHNOLOGIES, Les Ulis (FR)

(72) Inventors: Zahra KADRI, Fontenay-aux-Roses (FR); Stany CHRETIEN, Fontenay-aux-Roses (FR); Emmanuel PAYEN, Fontenay-aux-Roses (FR); Bruno YOU, L'HAY LES ROSES (FR); Celine DUCLOUX, ANTONY (FR)

(21) Appl. No.: 18/256,801

(22) PCT Filed: Dec. 13, 2021
(86) PCT No.: PCT/EP2021/085535

§ 371 Date: Jun. 9, 2023

(30) Foreign Application Priority Data
Dec. 11, 2020 (EP) 20306551.1

(51) Int. Cl. C12N 5/078; C12N 5/00; C12N 7/00; G01N 33/50

(52) U.S. Cl. CPC C12N 5/0641; C12N 5/0018; C12N 7/00; G01N 33/5044; C12N 2501/14; C12N 2750/14051; G01N 2333/015

(57) ABSTRACT

The present invention concerns a novel human erythroid progenitor cell line, wherein at least 90% of the cells are CD36⁺ CD44⁻CD71⁺; and wherein the cells:—do not express the gene encoding the receptor of Granulocyte-macrophage colony-stimulating factor (GM-CSF-R gene) or express GM-CSF-R gene at a lower level than the cells of human UT-7/Epo-S1 cell line; and—express the gene encoding the receptor of erythropoietin (Epo-R gene). The present invention also concerns the uses thereof for producing, detecting, or quantifying parvovims B19. The present invention allows the use of the cell lines for 1) a highly sensitive B19 infectious particles detection, and, 2) the efficient production of infectious B19 particles.

### TECHNICAL FIELD OF THE INVENTION

The present invention is in the field of cell lines with improved properties, and more particularly of human erythroid progenitor cell lines with increased permissivity to parvovirus B19 infection. The present Inventors have developed novel stable cell lines able to efficiently produce infectious B19 particles in vitro and allowing efficient, reliable and highly sensitive B19 infectious particle detection systems. These cell lines are particularly useful for the rapid and stable production of parvovirus B19 (in particular infectious parvoviral B19 particles) as well as for the efficient, reliable and highly sensitive detection of parvovirus B19 (in particular infectious parvoviral B19 particles). Importantly, these cell lines are more permissive and more sensitive to B19 parvovirus than the cell lines and populations classically used for producing and/or detecting B19 parvovirus (Primary CD36⁺ erythroid progenitor cells, KU812 cells, UT-7 cells, UT-7/Epo cells, UT-7/Epo-S1 cells). The present invention thus relates to human erythroid progenitor cell lines, wherein at least 90% of the cells are CD36⁺CD44⁻CD71⁺; and wherein the cells:
do not express the gene encoding the receptor of Granulocyte-macrophage colony-stimulating factor (GM-CSF-R gene) or express GM-CSF-R gene at a lower level than the cells of human UT-7/Epo-S1 cell line; and
express the gene encoding the receptor of erythropoietin (Epo-R gene).

The present invention also concerns the uses thereof for producing, detecting, or quantifying parvovirus B19.

### BACKGROUND ART

Human Parvovirus B19 (B19V), a member of the genus Erythroparvovirus of the Parvoviridae family, is a widespread virus that is pathogenic to humans. The genome of B19V is a linear 5.6-kb single-stranded DNA, packaged into a 23-28 nm non-enveloped icosahedral capsid (1). Replication occurs in the nucleus of infected cells, via a double-stranded replicative intermediate and a rolling hairpin mechanism. B19V infection has been associated with a wide spectrum of diseases, ranging from erythema infectiosum during childhood (known as the “fifth disease” and characterized by a common “slapped-cheek” rash), to arthropathies, severe anaemia and systemic manifestations involving the central nervous system, heart and liver, depending on the immune competence of the host [reviewed in (2)].

Productive B19V is restricted to human erythroid progenitor cells, and its clinical manifestations are linked to the destruction of infected cells (3). Acute B19V infection can cause pure red-cell aplasia in patients with pre-existing hematologic disorders leading to high levels of erythrocyte turnover (e.g. sickle cell disease or thalassemia patients), and in immunocompromised (e.g. cancer, HIV, acquired immunosuppression, chemotherapy, viral or parasitic or bacterial infection, etc.) or transplanted patients (4). The virus is transmitted via respiratory secretions and foetomaternal blood transfers. During pregnancy, infection with B19V can cause non-immune foetal hydrops, congenital anaemia, myocarditis and terminal heart failure, leading to spontaneous abortion or stillbirth of the foetus (5).

Infectious B19 particles may be present in the blood and cells of infected patients, whether symptomatic or not. The high prevalence of B19V infection in the general population and the large number of blood donations used in the manufacture of plasma-derived factor concentrates favour high levels of contamination.

Reducing the risk of B19V infection is mandatory for suppliers of blood-derived products worldwide. The elimination or absence of viruses must be assessed in processes for the production of plasma-derived medical products. To date, detection of the B19 virus has been done by assaying genetic material (i.e. NAT: Nucleic Acid Testing; 6). However, NAT does not allow the presence of infectious particles to be assessed. In addition, B19V DNA quantification can be inadequate: viral DNA can persist in the serum for months after acute infection, and its levels are therefore not necessarily correlated with infectivity (7). In addition, DNA quantification does not allow to distinguish infectious from non-infectious B19 genome (since non-infectious particles as well as naked viral DNA may be detected). The use of titration-based B19V infectivity assays is therefore essential. Moreover, the last few decades have seen the development of regenerative therapies based on Hematopoietic or Mesenchymal Stem Cells (HSC or MSC) from bone marrow and synovium donors, respectively. According to the guidelines ensuring clinical grade of human stem cells, one of the major safety concerns is detecting latent viruses in cell sources (8). Stem cells seem to act as a latent reservoir for B19 infection. If viral contamination is overlooked at initial screening, then the virus may be amplified during culture before transplantation, through the reactivation of latent B19V (9). For all these reasons, a practical and sensitive in vitro method for assessing B19V infectivity is required. However, efforts to develop such methods have been hampered by the lack of suitable B19-sensitive cell lines. In addition, there is currently no easy and efficient system for the production of infectious B19 particles. Yet, antiviral research requires native B19 infectious particles. But B19V particles from viremic patients limit the feasibility of high-throughput screening against the available chemical libraries. Animal (dog or pig) parvoviruses (CPV, PPV) are currently used to evaluate industrial procedures for viral reduction or inactivation. However, the physicochemical characteristics and behaviours of parvoviruses are not identical between species.

B19V displays a marked tropism for erythroid progenitor cells (EPC), but there is still no well-established cell line for B19V infection. Since the discovery that B19V inhibits erythroid colony formation in bone marrow cultures by inducing the premature apoptosis of erythroid progenitor cells, numerous approaches and studies attempt to find a method of virus culture in vitro. Primary (10) or immortalized (11 (WO2007/140011)) CD36+ erythroid progenitor cells (EPC) derived from hematopoietic stem cells were the most permissive cell models for B19V infection. CD36+ EPCs reflect the natural etiologic B19V cell host, but the main problem with the use of this model is the difficulty obtaining a continuously homogeneous cell line, with respect to differentiation stage, proliferation rate and metabolic activity. Moreover, the effectiveness of detection is limited by their low sensitivity. In addition, the reagents and cytokines required for cell culture (SCF, Il-3, Il-6, Epo) preclude the use of CD36+ EPCs for routine B19V cell-based detection methods. To counteract this lack of suitability, cancer cell lines constitute a sound, practical, cost-effective alternative model, overcoming these difficulties. During past years, many cancer cell lines have been tested. For example, the TF-1 cell line, a cell line derived from the bone marrow aspirate of an erythroleukemic patient (12) display marked erythroid morphological and cytochemical features common to CD36+ EPCs. The constitutive expression of globin genes highlights the commitment of the cells to the erythroid lineage (13). However, Gallinella et al. showed that TF-1 cells allow only B19V entry, with impaired viral genome replication and transcription, as shown by the presence of single-stranded DNA, and the absence of double-stranded DNA and RNA in B19V-infected TF-1 cells (14).

Only a few erythroid leukemic (KU812) (15) or megakaryoblastoid cell lines (UT-7) with erythroid characteristics support B19V replication. However, the percentage of B19V-positive cells was low (<1% immunofluorescent B19V+ cells (16).

Other studies have used another approach based on the amplification of erythroid progenitor cells (CD36⁺ EPC) from peripheral blood without mobilization or preselection. However, such cells have several drawbacks impacting reproducibility of the data, such as high heterogeneity between cells within the same cell culture and the need for human donors. In addition, primary cells are usually derived from different donors in order to obtain sufficient cell material, resulting in a high heterogeneity between different cell cultures. Moreover, CD36⁺ EPC expansion requires CD34⁺ purification, then expansion for at least a week, and finally differentiation to the erythroid lineage. All these steps prior to the use for assay or production of B19 are very technical and require expensive media and cytokines. Another constraint is that the CD36⁺ EPC cells are instable. Indeed, they cannot be maintained for prolonged time in the EPC stage, as they will continue to differentiate.

The UT7/Epo-S1 cell line (16), an erythropoietin (Epo)-dependent subclone derived from the megakaryoblastoid cell line UT-7 (17), is the most widely used cell model, because of its high sensitivity to B19V replication and transcription. However, B19V infection is limited to a small number of cells (1 to 9%, versus 30-40% for primary or immortalized erythroid progenitor cells) (18).

Yet, the production of native or recombinant infectious viral particles would make it possible to envisage their use for efficient and reliable detection of B19 in biological samples (in particular donated blood, plasma, tissues and organs), for diagnosing B19 infections, for antiviral research (e.g. identification of antiviral agents and compounds), to test industrial processes for viral reduction, and/or to envisage their use as a therapeutic vector in gene therapy. Therefore, there is a pressing need to provide novel and stable cell lines, having a high permissivity and sensitivity to B19 parvovirus infection.

The present invention fulfils this need. Indeed, the present Inventors have developed original and stable cell lines allowing 1) an efficient, reliable and more sensitive B19 particle detection system, 2) the stable and efficient production of infectious B19 particles in vitro.

The present invention thus provides novel, sensitive and efficient tools for detecting and amplifying infectious parvoviral B19 particles.

### DETAILED DESCRIPTION OF THE INVENTION

### Definitions

Unless specifically defined herein, all technical and scientific terms used herein have the same meaning as commonly understood by a skilled artisan in chemistry, biochemistry, cellular biology, molecular biology, and medical sciences.

As used herein, when used to define products, compositions, cell lines, uses and methods, the term “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are open-ended and do not exclude additional, unrecited elements or method steps. “Consisting of” means excluding any other components or steps “Consisting essentially of” means excluding other components or steps of any essential significance (however, other minor/insignificant components or steps are not excluded). In the present disclosure, the terms “comprising”, “consisting of” and “consisting essentially of” may be replaced with each other, if required.

The terms “virus” and “viral vector” are used interchangeably and are to be understood broadly as meaning a vehicle comprising at least one element of a wild-type virus genome that may be packaged into a viral particle or to the viral particle itself. These terms include viral vector (e.g. DNA viral vector) as well as viral particles generated thereof. Usually, a virus comprises a DNA or RNA viral genome packaged into a viral capsid and, in the case of an enveloped virus, lipids and other components (e.g. host cell membranes, etc). The terms “virus” and “viral vector” encompass wild-type and engineered viruses.

As used herein, “parvovirus” refers to a virus belonging to the Parvoviridae family of small, rugged, genetically-compact DNA viruses. There are currently more than 100 species in the family, divided among 23 genera in three subfamilies. Parvoviruses are linear, non-segmented, single-stranded DNA viruses, with an average genome size of 4-6 kilo base pairs (kbp). Parvoviruses are among the smallest viruses and are −20-30 nm in diameter. Parvovirus particles (virions) have a durable non-enveloped protein capsid in diameter that contains a single copy of the linear single-stranded DNA genome. The viral capsid of a parvovirus is made up of 60 copies of two or more size variants of a single protein sequence, called VP1, VP2 etc., which form a resilient structure with T=1 icosahedral symmetry. The linear single-stranded DNA genome in the capsid terminates in small imperfect palindromes that fold into dynamic hairpin telomeres. These terminal hairpins are hallmarks of the family, giving rise to the viral origins of DNA replication and mediating multiple steps in the viral life cycle including genome amplification, packaging, and the establishment of transcription complexes. However, they are often refractory to detection by PCR amplification strategies since they tend to induce polymerase strand-switching. Many parvoviruses are exceptionally resistant to inactivation, remaining infectious for months or years after release into the environment.

Parvoviruses encode at least two major gene complexes: the non-structural (or rep) gene that encodes the replication initiator protein (called NS1 or Rep), and the VP (or cap) gene, which encodes a nested set of −2-6 size variants derived from the C-terminus of the single VP protein sequence. Members of the Parvovirinae also encode a few (1-4) small genus-specific ancillary proteins that are variably distributed throughout the genome, show little sequence homology to each other, and appear to serve an array of different functions in each genus.

Parvoviruses are classified as group II viruses in the Baltimore classification of viruses. Parvoviruses can infect and may cause disease in many animals, from arthropods such as insects and shrimp, to echinoderms such as starfish, and to mammals including humans.

Parvoviruses that infect vertebrate hosts make up the subfamily Parvovirinae, while those that infect invertabrates (currently only known to infect insects, crustacea, and echinoderms) make up the subfamily Densovirinae. Prior to 2014, the name parvovirus was also applied to a genus within subfamily Parvovirinae, but this genus name has been amended to Protoparvovirus to avoid confusion between taxonomic levels.

Humans can be infected by viruses from five of the eight genera in the subfamily Parvovirinae: i) Bocaparvovirus (e.g. human bocavirus (HboV) 1-4), ii) Dependoparvovirus (e.g. adeno-associated virus (AAV) 1-5), iii) Erythroparvovirus (e.g. parvovirus B19 (B19V)), iv) Protoparvovirus (e.g. bufavirus (BuV) 1-3 (e.g. bufavirus 1a); cutavirus (CuV)), and v) Tetraparvovirus (e.g. human parvovirus 4 G1-3 (PARV4 G1-3). As of 2018, no known human viruses were in the remaining three recognized genera: vi) Amdoparvovirus (e.g. Aleutian mink disease virus), vii) Aveparvovirus (e.g. chicken parvovirus), and viii) Copiparvovirus (e.g. bovine parvovirus 2).

“Human Parvovirus B19” or “B19V” or “B19” or “parvovirus B19” or “Primate erythroparvovirus 1” or “erythrovirus B19” (those terms are herein synonymous) herein refers to a virus which is a member of the genus Erythroparvovirus of the Parvoviridae family. It is a widespread virus that is pathogenic to humans. B19 is a non-enveloped, icosahedral virus that contains a single-stranded linear DNA genome of approximately 5,600 base pairs in length. The infectious particles may contain either positive or negative strands of DNA. The icosahedral capsid consists of 60 capsomeres, consisting of two structural proteins, VP1 (83 kDa) and VP2 (58 kDa), which are identical except for 227 amino acids at the amino-terminal of the VP1-protein, the so-called VP1-unique region. VP2 is the major capsid protein, and comprises approximately 95% of the total virus particle. VP1-proteins are incorporated into the capsid structure in a non-stoichiometrical relation. the VP1-unique region is assumed to be exposed at the surface of the virus particle. At each end of the DNA molecule there are palindromic sequences which form “hairpin” loops. The hairpin at the 3′ end serves as a primer for the DNA polymerase. B19 is classified as an erythrovirus because of its capability to invade red blood cell precursors in the bone marrow. Three genotypes (1, 2 and 3) are recognised.

Parvovirus B19 is most known for causing disease in the pediatric population; however, it can also affect adults. It is the cause of the childhood rash called fifth disease or erythema infectiosum, or “slapped cheek syndrome”. B19V infection has been associated with a wide spectrum of other diseases, ranging from arthropathies (arthritis and arthralgias), severe and/or chronic anaemia, aplastic crisis, hydrops fetalis, and systemic manifestations involving the central nervous system, heart and liver, depending on the immune competence of the host.

Productive B19V is restricted to human erythroid progenitor cells (8), and its clinical manifestations are linked to the destruction of infected cells.

«Parvoviral B19 particle» or «B19 particle» herein means a complete parvovirus B19 particle (also known as a virion), and comprises at least (or consist essentially of, or consist of) nucleic acid surrounded by a protective coat of protein called a capsid.

«Infectious Parvoviral B19 particle» or «Infectious B19 particle» or «Infectious B19» herein refers to a B19 particle able to infect and enter into a host cell or subject. Advantageously, an infectious B19 is capable of completing an infectious cycle. An infectious cycle of parvovirus B19 involves (or consists essentially of, or consist of) binding to host cell receptors (e.g. human erythroid progenitor cell receptors), internalization, translocation of the genome to the host nucleus, DNA replication, RNA transcription, assembly of capsids and packaging of the genome.

The term “naturally occurring” or “native” or “wild type” is used to describe a biological molecule or organism that can be found in nature as distinct from being artificially produced by human. For example, a naturally occurring, native or wild-type virus refers to a virus (in particular parvovirus B19) which can be isolated from a source in nature (infected subject or infected tissue/cells from an infected subject) or which has previously been isolated from a source in nature and can now be obtained from specific collections (e.g. ECCAC, ATCC, CNCM, etc) in which it has been deposited. A biological molecule or an organism which has been intentionally modified by human intervention in the laboratory is not naturally occurring. Representative examples of “non-naturally occurring viruses” include, among many others, recombinant viruses.

As used herein, “recombinant parvovirus B19” means a parvovirus B19 which has been modified by the insertion of one or more nucleic acid(s) of interest (preferably a foreign nucleic acid, i.e. a nucleic acid originating from another species, also called recombinant nucleic acid, e.g. a recombinant gene) in B19 genome and/or a modified (e.g. defective) parvovirus B19 resulting from one or more modification(s) in the viral genome (e.g. total or partial deletion of a viral nucleic acid sequence (e.g. a viral gene), total or partial substitution of a viral nucleic acid sequence (e.g. a viral gene), or inactivation of a viral nucleic acid sequence (e.g. a viral gene) by one or more point substitution(s), insertion(s), and/or deletion(s)).The “foreign nucleic acid” that is inserted in the B19 genome is not found in, or expressed by, a naturally-occurring B19 genome. Nevertheless, the foreign nucleic acid can be homologous or heterologous to the cell or subject into which the recombinant B19 is introduced. More specifically, it can be of human origin or not (e.g. of animal, bacterial, yeast, or viral origin except B19). Advantageously, said foreign nucleic acid encodes a polypeptide or is a nucleic acid sequence capable of binding at least partially (by hybridization) to a complementary cellular nucleic acid (e.g., DNA, RNA, miRNA) present in a diseased cell with the aim of inhibiting a gene involved in said disease. A polypeptide is understood to be any translational product of a polynucleotide regardless of size, and whether glycosylated or not, and includes peptides and proteins. Such a foreign nucleic acid may be a native gene or portion(s) thereof (e.g. cDNA), or any variant thereof obtained by mutation, deletion, substitution and/or addition of one or more nucleotides.

“Recombinant parvoviral B19 particle” herein refers to a particle of a recombinant parvovirus B19.

In contrast, “native parvovirus B19” herein means an unmodified, wild-type, naturally occurring parvovirus B19. The native B19 is not recombinant. “Native parvoviral B19 particle” herein refers to an unmodified, wild-type, naturally occurring, not recombinant, parvoviral B19 particle.

The term “infection” refers to the transfer of the viral nucleic acid into a cell. Preferably, the viral nucleic acid is replicated and/or viral proteins are synthesized. More preferably, new viral particles are assembled.

As used herein, the terms “cell line” refer to a population of cells with a theoretically unlimited capacity for division, and stable after successive mitosis. The term “stable” used in the context of a cell or a cell line means that the cell/cell line is stable in culture (i.e. that the characteristics are maintained in the cells even after new generations have been produced (e.g. by mitosis), in particular the phenotypic characteristics).

The cells of a cell line have theoretically an unlimited capacity for division. The cells of a cell line can be cancer cells taken from a patient (such as HeLa cells) or cells derived thereof. They can also be artificially transformed by an oncogene (e.g. an immortalizing gene such as T from SV40 or artificially mutated for genes involved in the regulation of the cell cycle (such as the p53 protein): the cells are thus referred to as being immortalized cells. The immortalized cells can therefore be grown for prolonged periods in vitro.

The methods for generating immortalised cell lines are well-known to the person skilled in the art and do not need to be described in detail. They include, but are not limited to:
Artificial expression and/or introduction of a viral gene that partially deregulates the cell cycle (e.g., the adenovirus type 5 E1 gene, simian virus 40 large T antigen (SV40 large T), papillomaviruses E6 and E7, adenovirus E1A, Epstein-Barr virus, human T-cell leukaemia virus, herpesvirus saimiri, etc.);
Artificial expression and/or introduction of genes encoding key proteins involved in immortality (for example telomerase which prevents degradation of chromosome ends during DNA replication in eukaryotes, telomerase reverse transcriptase (e.g. hTERT)), of oncogenes, of mutant p53 gene, etc.

Artificial expression and/or introduction of viral gene(s), gene(s) encoding proteins involved in immortality, oncogene(s), mutant p53 gene(s) (etc), can be achieved by any technique known from the skilled person. Such techniques include, but are not limiting to, transforming, transfecting, transducing, infecting, or any combination thereof, the cell or the cell line, with a nucleic acid molecule (advantageously a vector (such as a plasmid, a viral vector, a lentiviral vector, etc.) comprising the viral gene(s), gene(s) encoding proteins involved in immortality, oncogene(s), mutant p53 gene(s) (etc) of interest.

As used herein, the terms “clonal cell line” or “clone” refers to a homogeneous population of cells descended from a single cell, containing the same genetic makeup, with a theoretically unlimited capacity for division, and stable after successive mitosis.

The terms “derived from” or “obtained from” or “originating” or “originate from” are used as synonyms to identify the original source of a component (e.g. a polypeptide, nucleic acid molecule, virus, etc) but is not meant to limit the method by which the component is made which can be, for example, by chemical synthesis, homologous recombination, recombinant means or any other means.

As used herein, “a cell permissive/sensitive to parvovirus B19 infection” refers to a cell allowing B19V infection and capable of hosting the entire infectious cycle of parvovirus B19 (i.e. until neo-production of infectious virions). The terms “permissive” and “sensitive” are herein synonymous. Cells permissive to B19 infection include erythroid progenitor cells found in bone marrow, blood or foetal liver, UT7/Epo cells, UT7/Epo-S1 cells, KU812Ep6 cells, the cell lines of the present disclosure, etc. A cell/cell line may alternatively be “semi-permissive to B19V”. In this case, the cell/cell line allows B19V infection and hosts a partial viral life cycle that allows production of, for example, only DNA, empty and/or defective particles.

As used herein, “permissivity to parvovirus B19 infection” or “sensitivity to parvovirus B19 infection” or “permissivity of a cell/cell line to parvovirus B19 infection” refers to the capacity of a cell to allow B19V infection and to host entire infectious cycle of parvovirus B19 (i.e. until neo-production of infectious virions). Similarly, “semi-permissivity to parvovirus B19 infection” or “semi-permissivity of a cell/cell line to parvovirus B19 infection” refers to the ability of a cell to allow B19V infection and to host a partial viral life cycle that allows production of, for example, only DNA, empty and/or defective particles.

Methods of assessing permissivity/sensitivity include, but are not limited to:
methods of visualization of particles entering the host cell (for example by immunofluorescence or fluorescence activated cell sorting (FACS) using specific antibodies directed against the capsid, or any method to detect and/or quantify the capsid proteins after short post-inoculation times);
methods of visualization of the inoculated B19 DNA in the host cell after short post-inoculation times, using any technique allowing to detect and/or quantify nucleic acids (in particular DNA);
methods allowing the detection and/or quantification of the neosynthesized B19 DNA in the host cell using any technique allowing to detect and/or quantify nucleic acids (in particular DNA; in this case, the quantity of B19 DNA measured after the step of cell infection is preferably compared to the quantity of B19 DNA measured before the step of cell infection and/or at the beginning of the infection);
methods allowing evaluation of the transcription of the B19V genome, using any technique allowing to detect and/or quantify nucleic acids (in particular transcripts (mRNAs and/or regulatory RNAs) and/or cDNA);
methods allowing evaluation of viral protein synthesis (NS or VP) within the host cell using any technique allowing to detect and/or quantify proteins;
methods allowing evaluation of post-infection cell lysis in susceptible cells (e.g. by counting the colony number before and after infection and counting the lysis plaque number);
methods allowing evaluation of neosynthesized infectious virions (e.g. Nucleic Acid Testing (NAT; such as analysis by NAT of culture supernatant (with or without a step cell lysis; DNA), analysis by NAT of infected cells (DNA), analysis by NAT after DNASE (enveloped DNA), analysis by NAT after infection of new cells, etc.) and any technique listed below); and
any combination thereof.

NAT is a technique that is routinely used by the skilled person and therefore there is no need to detail here. This technique is for instance described in detail in reference 6.

Methods for detecting and/or quantifying capsid proteins and techniques allowing to detect and/or quantify nucleic acids (such as DNA, cDNA, or RNA) and proteins are described below.

As used herein, “quantifying parvovirus B19” or “measuring/evaluating/determining the quantity/level/value of parvovirus B19” means the act/process of evaluating the quantity (also called a value or a level) of parvovirus B19 (in particular, based on its relationship to a quantity of the same species, taken as a unit and as a reference), from a value or quantity or signal measured and/or detected using a measuring (or quantifying or detecting) device and/or technique. Quantification of parvovirus B19 may comprise (consist essentially of, or consist of) detecting and quantifying parvovirus B19 nucleic acids (e.g. B19 genome, B19 cDNA, B19 transcripts (e.g. B19 mRNA and/or regulatory RNA), parvovirus B19 viral protein(s), parvovirus B19 viral particles (in particular infectious B19 viral particles), and any combination thereof. The quantity (or value, or level) can be a relative or an absolute quantity (or value, or level). For example, where the absolute quantity of a specific B19 protein or nucleic acid, or of a B19 particle, is to be determined, the reference unit may be one B19 protein molecule or one B19 nucleic acid molecule, or one B19 particle, respectively. For example, where the relative quantity of a specific B19 protein or nucleic acid, or of a B19 particle, is to be determined, the reference unit may be the quantity/value/level of a control/reference protein or nucleic acid (e.g. a B19 reference protein or nucleic acid, or a host cell reference protein or nucleic acid), or a control/reference cell, respectively.

The techniques and/or devices that may be used for detecting and measuring/quantifying B19 proteins, nucleic acids, or particles notably include well known analytical technologies.

At the protein level, B19 quantity can be measured, for example, by flow cytometry (in particular fluorescence activated cell sorting (FACS)), western blot, enzyme-linked immunosorbent assay, ELISA, ELISPOT, antibodies microarrays, immunoprecipitation, immunohistology, cell membrane staining using biotinylation or other equivalent techniques followed by immunoprecipitation with specific antibodies, dot blot, protein microarray, tissue microarray, antibody microarray, nucleic acid microarray, immunohistochemistry. Other suitable techniques include FRET or BRET, single cell microscopic or histochemistry methods using single or multiple excitation wavelength and applying any of the adapted optical methods, electrochemical methods (voltametry and amperometry techniques), atomic force microscopy, radio frequency methods (e.g. multipolar resonance spectroscopy), confocal and non-confocal microscopy, detection of fluorescence, luminescence, chemiluminescence, absorbance, reflectance, transmittance, and birefringence or refractive index (e.g., surface plasmon resonance, ellipsometry, resonant mirror methods, grating coupler waveguide methods, interferometry, etc.), cell ELISA, radioisotopic, magnetic resonance imaging, analysis by polyacrylamide gel electrophoresis (SDS-PAGE), HPLC, Mass Spectroscopy, Spectrometry, Chromatography coupled with (e.g. Liquid Chromatography/Mass Spectrometry/Mass Spectrometry (LC-MS/MS)), nucleic acid detecting/quantifying techniques.

The B19 quantity can be measured, for example, at the nucleotide level, by measuring the amount of B19 DNA and/or B19 transcripts (mRNA or regulatory RNA) and/or B19 cDNA. In such case, any technology usually used by the skilled person can be implemented. These technologies include well-known methods such as PCR (Polymerase Chain Reaction, if starting from DNA), RT-PCR (Reverse Transcription-PCR, if starting from RNA), quantitative RT-PCR (RT-qPCR), Nucleic Acid-Based Sensors, nucleic acid microarrays (including DNA chips and oligonucleotide chips), transcriptome analysis, RNA-seq analysis (including 3′RNASeq, 5′RNA-seq, 3′scRNA-Seq, 5′scRNA-Seq). Exemplary embodiments of RT-quantitative PCR are described in the experimental section. It is also possible to use hybridization with a labeled nucleic acid probe, such as northern blot (for mRNA) or Southern blot (for cDNA) or fluorescence in situ hybridization (FISH), but also by techniques such as the serial gene expression analysis method (SAGE) and its derivatives, such as LongSAGE, SuperSAGE, DeepSAGE, etc. It is also possible to use tissue chips (also known as TMAs: “tissue microarrays”). The tests usually used with tissue chips include immunohistochemistry and fluorescent in situ hybridization. For mRNA analysis, tissue chips can be coupled with fluorescent in situ hybridization. Finally, it is possible to use massive sequencing in parallel to determine the amount of mRNA in the sample (RNA-Seq or “Whole Transcriptome Shotgun Sequencing”).

At the level of B19 particles, B19 quantity can be measured, for example, by measuring the amount of B19 particles (infectious particles and/or non-infectious particles), using any of the techniques listed below for measuring infectious viral particles. More particularly, “quantifying infectious parvoviral BI 9 particles” means the act/process of evaluating the quantity (also called a value or a level) of infectious parvovirus B19. Any technique well-known in the art for detecting/quantifying infectious viral particles may be used. Such techniques include, but are not limited to: titration techniques (e.g. counting the number of plaques following infection of permissive cells), immunostaining (e.g. using anti-virus antibodies), spectrophotometry, quantitative immunofluorescence, quantification of encapsidated DNA by quantifying B19 DNA before and after DNAse treatment (using any technique for quantifying nucleic acids (in particular DNA) mentioned above), quantification of B19V positive cells after infection by quantifying viral protein, RNA or DNA in the host cells (using any technique for quantifying proteins or nucleic acids mentioned above; preferably after a step of cell purification, for example after removing the cell culture and washing the cells using an appropriate media) etc.

In addition, any of the above cited technologies can be used for detecting B19 (at the nucleic acid, protein and/or particle level) and infectious B19 particles, respectively.

It is possible to use a combination of one or more of any of the techniques listed above.

When the B19 is a recombinant B19, it is also possible to detect and/or measure/quantify B19 (at the nucleic acid, protein and/or particle level) and infectious B19 particles by detecting and/or measuring/quantifying any recombinant nucleic acid and/or protein expressed by the recombinant B19, using one or more of any of the techniques listed above.

All the above-listed technologies are routinely used by the skilled person and therefore there is no need to detail them here.

As used herein, “flow cytometry” or “FCM” is a technique used to detect and measure physical and chemical characteristics of a population of cells or particles. FCM is a useful tool for simultaneously measuring multiple physical properties of individual particles (such as cells, biomarkers, proteins, protein complexes, etc.). Cells pass single-file through a laser beam. As each cell passes through the laser beam, the cytometer records how the cell or particle scatters incident laser light and emits fluorescence. Using a flow cytometric analysis protocol, one can perform a simultaneous analysis of surface molecules at the single-cell level. The use of fluorescent agents or fluorochromes linked or attached to an antibody or antiserum able to specifically recognize a molecule or particle (such as a cell surface molecule (e.g. CD3, CD20, CD33, CD34, CD36, CD44, CD71, etc.) attached to a cell or portion thereof, a biomarker, a protein, a protein complex (e.g. calprotectin), etc., advantageously enables the flow cytometer to sort the molecules/particles on the basis of size, granularity and fluorescent light. Thus, the amount of information obtained from a single sample can be further expanded by using multiple fluorescent reagents. The information gathered by the flow cytometer can be displayed as any combination of parameters selected by the skilled person. For example, the flow cytometer can be configured to provide information about the relative size (forward scatter or “FSC”), granularity or internal complexity (side scatter or “SSC”), and relative fluorescent intensity of the cell sample.

The terms “selecting” or “cloning” herein refer to the action/process of producing individuals with essentially identical genome and/or DNA (preferably identical genome), either naturally or artificially. More particularly, selecting or cloning a cell or a cell line herein refers to the process of isolating one cell (or a population of cells having essentially identical genomes and/or DNA (preferably identical genomes)). The isolated cell can be expanded (i.e. grown in an appropriate medium, to allow cell division), until an appropriate number of cells is obtained (e.g. to obtain a cell line).

As used herein, the terms “erythroid progenitor cell” refer to committed self-renewing stem cells that give rise to erythrocytes (red blood cells).

As used herein, the terms “UT-7 cells” or “UT-7 cell line” refer to a pluripotent cell line established in 1990 from the bone marrow of a 64-year-old patient with acute megakaryoblastic leukemia (AML M7) by Dr. Komatsu's team (19). UT-7 cells growth and survival are strictly dependent on Il-3, GM-CSF, CFS, Epo or IL-6 (17). The cells of the UT-7 population are blocked by cancerous transformation to pluripotent hematopoietic progenitors and have markers of immature hematopoietic cells (CD34 and HLA-DR) on their surface. These cells have little differentiation and, depending on the cytokine used, develop differentiation markers specific to a given haematopoietic lineage). The UT-7 cells are commercially available from the catalogue of Leibniz Institute (DSMZ: Deutsche Sammlung von Mikroorganismen and Zellkulturen GmbH) under number DSMZ ACC-137. The UT-7 cell line is also referenced on Cellosaurus (https://web.expasy.org/cellosaurus/) under accession number CVCL_2233. From UT-7 cells, many cell lines have been developed (such cell lines may be referred to as sublines), based on their selection by exclusive growing under one specific hormone (such as UT-7/Epo, UT-7/GM, UT-7/Tpo, after selection under, respectively, Epo, GM-CSF and thrombopoietin (Tpo)). Specific cell lines, such as UT-7/Epo-S1, were also produced after isolation of a single clone (clonal cell lines)(16).

The terms “UT-7/Epo cells” or “UT-7/Epo cell line” refer to a cell line derived from the UT-7 cell line, wherein the cells have lost their capacity to differentiate under Epo, and are strictly dependent on Epo for growth. In contrast, the growth of UT-7/Epo is not supported by GM-CSF or IL-3. UT7/Epo cell line has been derived from the UT-7 cell line by maintenance of the UT-7 cell line in culture for more than 6 months in the presence of Epo. The UT-7/Epo cell line is referenced on Cellosaurus (https://web.expasy.org/cellosaurus/) under accession number CVCL_5202.

The terms “UT-7/Epo-S1 cells”, “UT-7/Epo-S1 cell line” or “UT-7/Epo-S1 clone” refer to a cell line derived from the UT-7/Epo cell line. UT-7/Epo-S1 was generated from a clone of UT-7/Epo, selected based on its high permissivity to B19V infection (16). This cell line is characterized by a high level of expression of GM-CSF-R (see FIG. 9B, showing higher expression than the UT-7/Epo-STI and UT-7/Epo-FUCCI cell lines and the E2 clone), a high level of phosphorylation of STAT5 when contacted with GM-CSF (see FIG. 9C, showing higher phosphorylation level than the UT-7/Epo-STI and UT-7/Epo-FUCCI cell lines), and low expression of Epo-R (see FIG. 9A showing lower expression than the UT-7/Epo-STI and UT-7/Epo-FUCCI cell lines and the E2 clone). This cell line shows some permissivity to B19V infection but much lower than the UT-7/Epo-STI and UT-7/Epo-FUCCI cell lines and the E2 clone (see FIG. 1B, FIG. 3 and FIG. 6A).

The terms “UT-7/GM cells” or “UT-7/GM cell line” refer to a cell line derived from the UT-7 cell line are dependent on GM-CSF for their growth. UT-7/GM cells have lost their capacity to proliferate under Epo, but can differentiate into erythroid cells after treatment with Epo (20). The UT-7/GM cell line is referenced on Cellosaurus (https://web.expasy.org/cellosaurus/) under accession number CVCL_5203.

The terms “UT-7/Epo-STI cells” or “UT-7/Epo-STI cell line” refer to a cell line obtained from UT-7/GM cells after a one-year period of maintenance and passage of UT-7/GM cells under erythropoietin (Epo) alone. These cells are strictly dependent on erythropoietin (Epo) for their proliferation and, unlike UT-7 and UT-7/GM cells, do not differentiate in its presence. These cells have a strong erythroid character and a cell morphology close to the pro-erythroblast. They express surface markers specific for erythroid differentiation (Epo receptor, CD36, CD71, CD117, Band3 . . . ) and erythroid-specific transcription factors (GATA-1, FOG-1). A basic cell bank (Master Bank) was established in 2002 in Port-Royal, and in 2008 at the CEA in Fontenay-Aux-Roses. Since then, working banks (Working Bank or WB) have been established on a regular basis according to needs. The characteristics of the cells are checked at each WB establishment: 1) Test for the presence of mycoplasma, 2) strict dependence on Epo for their growth, 3) absence of spontaneous haemoglobinisation under Epo, 4) haemoglobinisation and morphological changes under chemical induction. The WB used for parvovirus infection studies is WB2015 (noted UT7/Epo-STI). This cell line is characterized by a low level of expression of GM-CSF-R (see FIG. 9B, showing lower expression than the UT-7/Epo-S1 cell line), a low level of phosphorylation of STAT5 when contacted with GM-CSF (see FIG. 9C, showing lower phosphorylation level than the UT-7/Epo-S1 cell line), and higher expression of Epo-R compared to the UT-7/Epo-S1 cell line (see FIG. 9A). This cell line shows higher permissivity to B19V infection than the prior art UT-7/Epo-S1 cell line (see FIG. 1B and FIG. 3 showing permissivity to B19V about 9-10 times higher than the UT-7/Epo-S1 cell line). The UT-7/Epo-STI cell line has been deposited under the provisions of Budapest treaty, at the Collection Nationale de Cultures de Microorganismes (CNCM, having the address: CNCM, Institut Pasteur, 25 rue du Docteur Roux, F-75724 Paris Cedex 15), on 5 Oct. 2020, under the deposit number CNCM I-5599.

“Erythropoietin” or “haematopoietin” or “haemopoietin” or “Epo” is a glycoprotein cytokine secreted mainly by the kidney in response to cellular hypoxia. Epo is highly glycosylated (40% of total molecular weight). It stimulates red blood cell production (erythropoiesis) in the bone marrow. Erythropoietin is the primary erythropoietic factor that cooperates with various other growth factors (e.g., IL-3, IL-6, glucocorticoids, and SCF) involved in the development of erythroid lineage from multipotent progenitors. The amino acid sequence of human Epo is available under NCBI accession numbers AAI43226.1 or NP_000790.2 or EAW76494.1 or EAW76493.1.

As used herein, the terms “erythropoietin receptor” or “receptor of erythropoietin” or “Epo receptor” or “Epo-R” refer to the receptor for Epo. Epo-R is a 52kDa peptide with a single carbohydrate chain resulting in a n approximately 56-57 kDa protein found on the surface of Epo responding cells. It is a member of the cytokine receptor family. Epo-R pre-exists as dimers which upon binding of a 30 kDa ligand erythropoietin (Epo), changes its homodimerized state. The amino acid sequence of human Epo-R is for example available under NCBI accession number AAB23271.1.

“Granulocyte-macrophage colony-stimulating factor” or “GM-CSF” or “colony-stimulating factor 2” or “CSF2” is a monomeric glycoprotein secreted by macrophages, T cells, that functions as a cytokine. It is a white blood cell growth factor. GM-CSF stimulates stem cells to produce granulocytes (neutrophils, eosinophils, and basophils) and monocytes. GM-CSF signals via Signal Transducer and Activator of Transcription 5 (STAT5). The amino acid sequence of human GM-CSF is available under NCBI accession numbers AAA52578.1 or P04141.1.

As used herein, the terms “Granulocyte-macrophage colony-stimulating factor receptor” or “receptor of GM-CSF” or “GM-CSF receptor” or “GM-CSF-R” or “CD116” refer to the receptor for GM-CSF. GM-CSF-R is a heterodimer composed of at least two different subunits; an α chain, and a β chain. The a subunit contains a binding site for granulocyte macrophage colony-stimulating factor. The β chain is involved in signal transduction. Association of the α and β subunits results in receptor activation. The amino acid sequence of the α chain of human GM-CSF-R is for example available under NCBI accession numbers AAH71835.1 or AAH02635.1 or XP_011544467.1 or NP_001366085.1. The amino acid sequence of the β chain of human GM-CSF-R is for example available under NCBI accession numbers P32927.2 or NP_000386.1.

“Signal Transducer and Activator of Transcription 5” or “STAT-5” refers to two highly related proteins, STAT5A and STAT5B, which are part of the seven-membered STAT family of proteins. Though STAT5A and STAT5B are encoded by separate genes, the proteins are 90% identical at the amino acid level. STAT5 proteins are involved in cytosolic signaling and in mediating the expression of specific genes. Aberrant STAT5 activity has been shown to be closely connected to a wide range of human cancers, and silencing this aberrant activity is an area of active research in medicinal chemistry. The STAT5 signaling pathway is activated by fixation of Epo on Epo receptor or of GM-CSF on GM-CSF receptor. In order to be functional, STAT5 proteins must first be activated by phosphorylation. This activation is carried out by kinases associated with transmembrane receptors. pSTAT5 (phosphorylated STAT5) has been shown to facilitate B19 DNA replication in erythroid progenitors. The amino acid sequence of human Stat5A is available under NCBI accession numbers AAB06589.1 or NP_001275649.1 or NP_001275648.1 or NP_001275647.1. The amino acid sequence of human Stat5B is available under NCBI accession numbers AAH20868.1 or NP_036580.2 or P51692.2 or EAW60817.1.

As used herein, “CD3” or “cluster of differentiation 3” is a protein complex and T cell co-receptor that is involved in activating both the cytotoxic T cell (CD8+ naive T cells) and T helper cells (CD4+ naive T cells). It is composed of four distinct chains. In mammals, the complex contains a CD3γ chain, a CD3δ chain, and two CD3ε chains. These chains associate with the T-cell receptor (TCR) and the ζ-chain (zeta-chain) to generate an activation signal in T lymphocytes. The TCR, ζ-chain, and CD3 molecules together constitute the TCR complex. The amino acid sequences of human CD3γ chain, CD3δ chain, CD3ε chain are respectively available under NCBI accession numbers NP_000064.1 (CD3γ), NP_000723.1 (or NP_001035741.1; CD3δ), 1XIW_E (or 1XIW_A; CD3ε).

As used herein, “CD20” or “cluster of differentiation 20” or “B-lymphocyte antigen CD20” is a member of the membrane-spanning 4A protein family. Members of this protein family are characterized by common structural features and similar intron/exon splice boundaries and display unique expression patterns among hematopoietic cells and nonlymphoid tissues. CD20 is a B-lymphocyte surface molecule that plays a role in the development and differentiation of B-cells into plasma cells. The amino acid sequences of human CD20 is available under NCBI accession numbers NP_068769.2 or NP_690606.1 or NP_690605.1.

As used herein, “CD33” or “cluster of differentiation 33” or “Siglec-3” or “sialic acid binding Ig-like lectin 3” or “gp67” or “p67” is a transmembrane receptor expressed on cells of myeloid lineage. It binds sialic acids, therefore is a member of the SIGLEC family of lectins. The extracellular portion of this receptor contains two immunoglobulin domains (one IgV and one IgC2 domain). The intracellular portion of CD33 contains immunoreceptor tyrosine-based inhibitory motifs (ITIMs) that are implicated in inhibition of cellular activity. CD33 can be stimulated by any molecule with sialic acid residues such as glycoproteins or glycolipids. Upon binding, the immunoreceptor tyrosine-based inhibition motif (ITIM) of CD33, present on the cytosolic portion of the protein, is phosphorylated and acts as a docking site for Src homology 2 (SH2) domain-containing proteins like SHP phosphatases. This results in a cascade that inhibits phagocytosis in the cell. The amino acid sequences of human CD33 is available under NCBI accession numbers NP_001171079.1 or NP_001763.3 or NP_001076087.1.

As used herein, “CD34” or “cluster of differentiation 34” is a transmembrane phosphoglycoprotein protein that functions as a cell-cell adhesion factor. It may also mediate the attachment of hematopoietic stem cells to bone marrow extracellular matrix or directly to stromal cells. The CD34 protein is a member of a family of single-pass transmembrane sialomucin proteins that show expression on early hematopoietic and vascular-associated tissue. The amino acid sequences of human CD34 is available under NCBI accession numbers AAB25223.1 or AAB25222.1 or AAA03181.1.

As used herein, “CD36” or “cluster of differentiation 36” or “platelet glycoprotein 4” or “fatty acid translocase (FAT)” or “scavenger receptor class B member 3 (SCARB3)” or “glycoproteins 88 (GP88), IIIb (GPIIIB), or IV (GPIV)” is an integral membrane protein found on the surface of many cell types in vertebrate animals (such as platelets, erythrocytes, monocytes, differentiated adipocytes, skeletal muscle, mammary epithelial cells, spleen cells and some skin microdermal endothelial cells). It imports fatty acids inside cells and is a member of the class B scavenger receptor family of cell surface proteins. The amino acid sequences of human CD36 is available under NCBI accession number CAA83662.1.

As used herein, “CD44” or “cluster of differentiation 44” is a cell-surface glycoprotein involved in cell-cell interactions, cell adhesion and migration. CD44 has been referred to as HCAM (homing cell adhesion molecule), Pgp-1 (phagocytic glycoprotein-1), Hermes antigen, lymphocyte homing receptor, ECM-III, and HUTCH-1. CD44 participates in a wide variety of cellular functions including lymphocyte activation, recirculation and homing, haematopoiesis, and tutor metastasis. CD44 is a receptor for hyaluronic acid and can also interact with other ligands, such as osteopontin, collagens, and matrix metalloproteinases (MMPs). CD44 function is controlled by its posttranslational modifications. The amino acid sequences of human CD44 is available under NCBI accession number AC146596.1 or NP_000601.3 or NP_001001389.1.

As used herein, “CD71” or “cluster of differentiation 71” or “Transferrin receptor protein 1” or “TfR1” is a transmembrane glycoprotein composed of two disulfide-linked monomers joined by two disulfide bonds. Each monomer binds one holo-transferrin molecule creating an iron-Tf-TfR complex which enters the cell by endocytosis. The amino acid sequences of human CD71 is available under NCBI accession number NP_003225.2 or NP_001300894.1 or NP_001300895.1.

As used herein, “Integrin-α5” or “Integrin alpha-5” or “ITGA5” or “CD49e” or “cluster of differentiation 49e” is a protein belonging to the integrin alpha chain family. Integrins are heterodimeric integral membrane proteins composed of an alpha chain and a beta chain. Alpha chain 5 undergoes post-translational cleavage in the extracellular domain to yield disulfide-linked light and heavy chains that join with beta 1 to form a fibronectin receptor. In addition to adhesion, integrins are known to participate in cell-surface mediated signaling. The amino acid sequences of human Integrin-α5 (CD49e) is available under NCBI accession number NP_002196.4, or EAW96781.1, or EAW96780.1, or AAH08786.1.

A “CD3⁺ cell” is a cell that expresses CD3 at the cell surface (e.g. a cell wherein CD3 can be detected at the cell surface using any suitable analytical technology including FACS, immunofluorescence, immunohistochemistry, etc.). Similarly, a CD20⁺ cell, a CD33⁺ cell, a CD34⁺ cell, a CD36⁺ cell, a CD71⁺ cell, or a CD49e⁺ cell, is a cell that expresses at the cell surface CD20, CD33, CD34, CD36, CD71, or CD49e, respectively (e.g. a cell wherein CD20, CD33, CD34, CD36, CD71, or CD49e, respectively, can be detected at the cell surface using any suitable analytical technology mentioned above). In contrast, a “CD44⁻ cell” is a cell that does not expresses CD44 at the cell surface (e.g. a cell wherein CD44 cannot be detected at the cell surface using any suitable analytical technology mentioned above, etc).

As used herein, “cell cycle” or “cell-division cycle” refers to the series of events that take place in a cell that cause it to divide into two daughter cells. These events include the duplication of its DNA (DNA replication) and some of its organelles, and subsequently the partitioning of its cytoplasm and other components into two daughter cells in a process called cell division. In cells with nuclei (eukaryotes), (i.e., animal, plant, fungal, and protist cells), the cell cycle is divided into two main stages: the interphase and the mitotic (M) phase (including mitosis and cytokinesis). During interphase, the cell grows, accumulating nutrients needed for mitosis, and replicates its DNA and some of its organelles. During the mitotic phase, the replicated chromosomes, organelles, and cytoplasm separate into two new daughter cells. To ensure the proper replication of cellular components and division, there are control mechanisms known as cell cycle checkpoints after each of the key steps of the cycle that determine if the cell can progress to the next phase. Each phase of the cell cycle has a distinct set of specialized biochemical processes that prepare the cell for initiation of the cell division.

The eukaryotic cell cycle consists of four distinct phases: G1 phase (or Gap 1 phase), S phase (or Synthesis phase), G2 phase (or Gap 2 phase, also known as interphase) and M phase (or Mitosis phase, includes mitosis and cytokinesis). M phase is itself composed of two tightly coupled processes: mitosis, in which the cell's nucleus divides, and cytokinesis, in which the cell's cytoplasm divides forming two daughter cells. Activation of each phase is dependent on the proper progression and completion of the previous one.

Cells that have temporarily or reversibly stopped dividing are said to have entered a state of quiescence called GO phase (or Gap 0 phase).

In “G1 phase”, cells increase in size. The G1 checkpoint control mechanism ensures that everything is ready for DNA synthesis. It is the first phase within interphase, from the end of the previous M phase until the beginning of DNA synthesis. It is also called the growth phase. During this phase, the biosynthetic activities of the cell, which are considerably slowed down during M phase, resume at a high rate. The duration of G1 is highly variable, even among different cells of the same species. It is usually the longest phase. It can be divided into early G1 (eG1) and late G1. In G1 phase, the cell increases its supply of proteins, increases the number of organelles (such as mitochondria, ribosomes), and grows in size. In G1 phase, a cell has three options: (i) Continue cell cycle and enter S phase; (ii) Stop cell cycle and enter GO phase for undergoing differentiation; (iii) Become arrested in G1 phase hence it may enter GO phase or re-enter cell cycle. The deciding point is called check point (Restriction point). This check point is called the restriction point or START and is regulated by G1/S cyclins, which cause transition from G1 to S phase. Passage through the G1 check point commits the cell to division.

In “S phase”, DNA replication occurs. The S phase starts when DNA synthesis commences. When it is complete, all of the chromosomes have been replicated, i.e., each chromosome consists of two sister chromatids. Thus, during this phase, the amount of DNA in the cell has doubled, though the ploidy and number of chromosomes are unchanged. Rates of RNA transcription and protein synthesis are very low during this phase. An exception to this is histone production, most of which occurs during the S phase.

In “G2 phase”, the cell will continue to grow. The G2 checkpoint control mechanism ensures that everything is ready to enter the M (mitosis) phase and divide. G2 phase occurs after DNA replication and is a period of protein synthesis and rapid cell growth to prepare the cell for mitosis. During this phase microtubules begin to reorganize to form a spindle (preprophase). Before proceeding to mitotic phase, cells must be checked at the G2 checkpoint for any DNA damage within the chromosomes. The G2 checkpoint is mainly regulated by the tumour protein p53.

In “M phase”, cell growth stops and cellular energy is focused on the orderly division into two daughter cells. The mitotic phase of M phase consists of mitosis and nuclear division (karyokinesis). It is a relatively short period of the cell cycle, yet complex and highly regulated. A checkpoint in the middle of mitosis (Metaphase Checkpoint) ensures that the cell is ready to complete cell division. Mitosis is the process by which a eukaryotic cell separates the chromosomes in its cell nucleus into two identical sets in two nuclei. During the process of mitosis, the pairs of chromosomes condense and attach to microtubules that pull the sister chromatids to opposite sides of the cell. The sequence of mitosis events is divided into phases, sequentially known as prophase, prometaphase, metaphase, anaphase, and telophase. The mitotic phase is immediately followed by cytokinesis, which divides the nuclei, cytoplasm, organelles and cell membrane into two cells containing roughly equal shares of these cellular components.

A “cell cycle indicator” as used herein is a substance and/or a molecule allowing to detect at least one phase of the cell cycle in a living cell, cell line or cell population. Examples of cell cycle indicators include analogues of nucleotides that incorporate into DNA and are revealed by immunostaining (BrDU, EDU), chemical fluorescent DNA dye (Hoechst, Pyronine Y) and fluorescent cell cycle indicators. Examples of fluorescent cell cycle indicators include chemical fluorescent dye (Hoechst for example), Fluorescent Ubiquitination Cell Cycle Indicator (FUCCI). A fluorescent cell cycle indicator can be a fluorescent protein, encoded by a reporter gene which is expressed only during one, two, or three of the 4 main phases (G1, S, G2, M) of the cell cycle (for example by placing the gene under the control of promoter(s) activated only during either G1 or S or G2 or M phases or by introducing a cell cycle-dependent suppressing sequence within the nucleic acid of the reporter gene or corresponding transcript and/or the amino acid sequence of the protein encoded by the reporter gene). It is possible to combine more than one indicator (e.g. two, three, four or more indicators for the same or for distinct cell cycle phases). It is also possible to use an indicator allowing to visualise more than one cell cycle phase (e.g. an indicator of all S, G2 and M phases).

As used herein, “Fluorescent Ubiquitination Cell Cycle Indicator” or “Fluorescent Ubiquitination-based Cell Cycle Indicator” or “FUCCI” or “FUCCI system” is a set of fluorescent probes which enables the visualization of cell cycle progression in living cells. FUCCI may notably use the phase-dependent nature of various cell proteins, such as replication licensing factors. In this case, FUCCI uses the highly selective, rapid degradation of the replication licensing factors mediated by the ubiquitin proteasome system to give excellent visualizations of the cell cycle. Examples of replication licensing factors include Cdt1 and Geminin. To allow detection, the replication licensing factor or fragment thereof can be fused to a fluorescent protein or probe. For example, a fusion protein of a fragment of Cdt1 (amino acids 30-120) with a fluorescent protein may serve as an indicator of G1 phase. For example, a fusion protein of a fragment of Geminin (amino acids 1-110 or 1-60) with a fluorescent protein (provided the fluorescent protein is distinct from and its fluorescence may be distinguished from that of the fluorescent protein fused to Cdt1, when Cdt1 and Geminin fusion proteins are both used) may visualize the S, G2 and M phase. Examples of a fluorescent proteins include Green Fluorescent Protein (GFP), Yellow Fluorescent Protein (YFP), Red Fluorescent Protein (RFP), Cyan Fluorescent Protein (CFP), mCherry, mVenus, mApple, Renilla, monomeric Kusabira-Orange 2 (mKO2), monomeric Azami-Green 1 (mAG1), mKate2, mTurquoise, etc.

“Cdt1” or “Cdc10 dependent transcript 1” or “Chromatin licensing and DNA replication factor 1” is a conserved replication factor required for licensing the chromosome for a single course of DNA synthesis. It is a key licensing factor in the assembly of pre-replication complexes (pre-RC), which occurs during the G1 phase of the cell cycle. From the onset of S phase through mitosis, Cdt1 is inhibited by several pathways to prevent relicensing, thus ensuring that DNA is only replicated once per cell cycle. For example, during the S, G2 and M phases, Cdt1 is inhibited by the protein Geminin through ubiquitination of Cdt1 by the ubiquitin ligase complex SCFskp2 and degradation by the proteasome. A cell expressing a fluorescent protein fused to CdT1 or fragments thereof (in particular amino acids 30-120 of Cdt1) will thus be fluorescent only during G1 phase. In human, Cdt1 has the NCBI reference protein sequence BAB61878.1 or P_112190.2.

“Geminin” or “DNA replication inhibitor” is a nuclear protein made up of about 200 amino acids, with a molecular weight of approximately 25 kDa. It contains an atypical leucine-zipper coiled-coil domain. Geminin is absent during G1 phase and accumulates through S, G2 phase and M phases of the cell cycle. At the start of the S-phase until late mitosis, geminin inhibits the replication factor Cdt1, preventing the assembly of the pre-replicative complex. In early G1, the APC/C complex triggers Geminin destruction through ubiquitination. A cell expressing a fluorescent protein fused to Geminin or fragments thereof (in particular amino acids 1-110 or 1-60 of Geminin) will thus be fluorescent during all of S, G2 and M phases. The amino acid sequence of human Geminin is available under NCBI accession numbers NP_056979.1 or NP_001238919.1 or NP_001238918.1 or NP_001238920.1.

The terms “UT7/Epo-FUCCI cells” or “UT-7/Epo-FUCCI cell line” refer to a cell line obtained after stable expression of FUCCI system by transduction of UT-7/Epo-STI cells with FUCCI lentiviral particles. This cell line is characterized by absence of expression of GM-CSF-R (see FIG. 9B), absence of phosphorylation of STAT5 when contacted with GM-CSF (see FIG. 9C), and higher expression of Epo-R compared to the UT-7/Epo-S1 cell line (see FIG. 9A). This cell line shows higher permissivity to B19V infection than the prior art UT-7/Epo-S1 cell line (see FIG. 6A showing permissivity to B19V about 5 times higher than the UT-7/Epo-S1 cell line). It is further characterized by the fact that a majority of the cells are in the S/G2/M phases of the cell cycle (see FIG. 5 and FIG. 6B).

The terms “clone E2 obtained from UT-7/Epo-FUCCI cell line” or “UT7/Epo-STI-derived clone E2” or “clone E2” or “UT-7/Epo-E2 cells” or “UT-7/Epo-E2 cell line” refer to a cell line obtained from the clone E2, which has been isolated from the UT-7/Epo-FUCCI cell line and selected for its high permissivity to B19V infection (see FIG. 6A showing permissivity to B19V more than 30 times higher than the UT-7/Epo-S1 cell line). This clone is further characterized by absence of expression of GM-CSF-R (see FIG. 9B), higher expression of Epo-R compared to the UT-7/Epo-S1 cell line (see FIG. 9A), and a high percentage of cells in the S/G2/M phases of the cell cycle (see FIG. 5 and FIG. 6B).

The clone E2 has been deposited under the provisions of Budapest treaty, at the Collection Nationale de Cultures de Microorganismes (CNCM, having the address: CNCM, Institut Pasteur, 25 rue du Docteur Roux, F-75724 Paris Cedex 15), on 5 Oct. 2020, under the deposit number CNCM I-5600.

As used herein, a “viral reduction process” is a process aiming at reducing (preferably eliminating) the quantity or infectivity of a virus (preferably a parvovirus B19) in a sample (such as a biological sample). Viral reduction processes include “viral elimination processes” in which at least part of the virus is removed (such as nanofiltration techniques) and “viral inactivation processes” in which at least part of the virus is inactivated, i.e. its infectivity is reduced or destroyed (such as pasteurisation, dry heating, or solvent-detergent treatment in the case of enveloped viruses).

As used herein, the term “screening” refers to a process for testing and selecting compounds/active agents for a specific effect/activity on a molecule, a virus, a parasite, a bacterium, a cell, a tissue, an organ, an organism (human beings, human embryos and human embryonic stem cells excluded). For example, the compounds/active agents may be tested for an antiviral activity/effect, such as an anti-parvovirus B19 activity/effect.

As used herein, a “subject” or an “individual” is an animal, preferably a mammal, including, but not limited to, human, dog, cat, cattle, goat, pig, swine, sheep and monkey. More preferably, the subject is a human subject. A human subject can be known as a patient. As used herein, “subject of interest” refers to any one of:
a subject that may be infected with, or that is susceptible of being infected with, or that is suspected of being infected with, or that has been diagnosed as being infected with, parvovirus B19;
a subject suffering from haematological deficiency, such as immune depression (cancer, HIV, acquired or induced immunosuppression (i.e. before transplantation or chemotherapy)) or failure to produce red blood cells of constitutive origin (red-cell aplasia, thalassemia, sickle cell disease, constitutional or acquired mutations . . . ) or external origin (viral, parasitic or bacterial) origin, a pregnant subject;
a subject that may need, or is susceptible to need, or is suspected of needing, a transplant;
a donor subject (donating a biological sample, such as an organ, a tissue, cells, blood, plasma, serum, labile blood products (such as platelet concentrate or red blood cell concentrate), synovium, bone marrow), in particular before administration/transplant to another subject in need of administration/transplant of the donated biological sample or part thereof).

A “healthy subject” refers to a subject, that is not infected with, or that is not susceptible of being infected with, or that is not suspected of being infected with, or that has not been diagnosed as being infected with, parvovirus B19. In particular, the “healthy subject” does not suffer from any disease, or has not been diagnosed with any disease.

A “control subject” or a “reference subject” is a subject that may either be a healthy subject or a subject suffering from a disease (preferably a parvovirus B19 infection) at a specific stage (e.g. an asymptomatic, or a benign, or a mild, or an acute/severe parvovirus infection), which is used as a positive or negative control/reference in any test/assay.

As used herein, the terms “biological sample” or “sample” refer to an entire organ or tissue or fluid (e.g. blood, serum, plasma, milk, etc.) of one or more subject(s), or cells or cell components thereof (e.g. organelles, nucleic acids, proteins, etc.), or a fraction of tissue, organ, fluid, or cell, or a homogenate, a lysate or a crude or purified extract prepared from an entire organ or tissue or fluid of one or more subject(s), or cells or cell components thereof, or a fraction of tissue, organ, fluid, or cell. In particular, a “biological sample” or “sample” may be any tissue or fluid which may contain B19 parvovirus including, but not limited to, a blood, plasma, serum, labile blood products (such as platelet concentrate or red blood cell concentrate), synovium, bone marrow, and any combination thereof. In particular, the biological sample may be from a subject of interest.

As used herein, “diagnosis” or “identifying a subject having”, or “diagnosing” refers to a process of determining if a subject is afflicted with a disease, condition or ailment (in particular a B19 infection). “Diagnosis” means the identification/determination of the presence or absence of a disease (or condition, or ailment) in a subject. Diagnosis includes, for example, the investigation of the causes (etiology) and effects (symptoms) of the disease, in particular on the basis of observations and/or measurements, carried out using various tools.

More specifically, “diagnosing B19 infection” refers to the process of determining whether a subject is infected with B19.

### Cell Lines of the Invention

The present Inventors have developed novel and stable cell lines that are highly permissive to parvovirus B19 infection (see FIG. 1B, FIG. 3 and FIG. 6A). These cell lines are derived from a human erythroid progenitor cell line (UT-7/GM cell line, itself derived from the UT-7 cell line). The Inventors have shown that these cell lines allow an efficient, reliable and more sensitive B19 particle detection system, as well as a stable and efficient production of infectious B19 particles in vitro. More precisely, comparative data surprisingly showed that these novel cell lines are significantly more permissive to B19 infection than the cell populations/lines classically used for detecting and/or producing B19 (see FIG. 1B, FIG. 3 and FIG. 6A). In addition, these cell lines are homogeneous and stable, contrary to primary erythroid progenitor cells. Importantly, the Inventors have shown that these cell lines are characterized by absence of expression or low expression the GM-CSF-R (see FIG. 9B, resulting in low or absent phosphorylation of STAT5 when contacted with GM-CSF, see FIG. 9C) and high expression of Epo-R (see FIG. 9A), strict dependence on Epo for their growth, providing new selection criteria for the isolation of further cell lines with high permissivity to B19. The inventors have also demonstrated for the first time a direct correlation between infectivity and the response to the cytokine GM-CSF, which further provides another new selection criterium for the isolation of further cell lines with high permissivity to B19.

Accordingly, the present invention relates to a human erythroid progenitor cell line, wherein at least 90% of the cells are CD36⁺CD44⁻CD71⁺; and wherein the cells:
do not express the gene encoding the Receptor of Granulocyte-Macrophage Colony-Stimulating Factor (GM-CSF-R gene), or express low levels of GM-CSF-R gene; and
express the gene encoding the Receptor of Erythropoietin (Epo-R gene).

In addition, the present invention relates to a human erythroid progenitor cell line, wherein at least 90% of the cells are CD36⁺CD44⁻CD71⁺; and wherein the cells:
do not express the gene encoding the Receptor of Granulocyte-macrophage colony-stimulating factor (GM-CSF-R gene) or express GM-CSF-R gene at a lower level than the cells of human UT-7/Epo-S1 cell line; and
express the gene encoding the Receptor of Erythropoietin (Epo-R gene).

Indeed, the data obtained by the Inventors show that the cells of the cell line of the invention express significantly low levels of GM-CSF-R gene (in particular the cells express GM-CSF-R gene at a lower level than the cells of human UT-7/Epo-S1 cell line (as UT-7/Epo-STI, see FIG. 9B)), or do not express GM-CSF-R gene (as clone E2, see FIG. 9B). The data demonstrate that this low expression or this absence of expression of GM-CSF-R is correlated with an increased permissivity to B19V infection.

In addition, the level of response of the cell line of the invention to cytokine GM-CSF is very low (or even null in some embodiments, see FIG. 9C), at least due to this low expression or this absence of expression of GM-CSF-R. The Inventors have in particular demonstrated for the first time that an increased permissivity to B19V is correlated to an absence of response of the cells to the cytokine GM-CSF. Thus, the present invention also relates to a human erythroid progenitor cell line, wherein at least 90% of the cells are CD36⁺CD44⁻CD71⁺; wherein the cells are not responsive to the Granulocyte-Macrophage Colony-Stimulating Factor (GM-CSF) cytokine (in particular they do not phosphorylate STAT5 or phosphorylate STAT5 at a low level, lower than the UT-7/Epo-S1 cell line, when contacted with GM-CSF); and wherein the cells express the gene encoding the Receptor of Erythropoietin (Epo-R gene).

In an advantageous embodiment, at least 91% of the cells of the cell line of the invention are CD36⁺CD44⁻CD71⁺, more preferably at least 92%, more preferably at least 93%, more preferably at least 94%, more preferably at least 95%, more preferably at least 96%, more preferably at least 97%, more preferably at least 98%, more preferably at least 99% of the cells are CD36⁺CD44⁻CD71⁺, even more preferably the cells are CD36⁺CD44⁻CD71⁺.

The cells of the cell line of the invention preferably express high levels of the gene encoding the Receptor of Erythropoietin (Epo-R gene), in particular at a higher level than the cells of human UT-7/Epo-S1 cell line. Indeed, the data demonstrate that the cells express the gene encoding Epo Receptor (Epo-R), and at a significantly higher level than the cells of human UT-7/Epo-S1 cell line (see FIG. 9A).

In a preferred embodiment, the level of expression of GM-CSF-R gene and/or Epo-R gene is(are) detected and/or quantified by conventional methods, such as Reverse Transcription Polymerase Chain Reaction (RT-PCR), RT quantitative PCR (RT-qPCR), western blot, and any combination thereof. Indeed, the data show that, by using such conventional methods, the cells of the cell line of the invention express low levels of GM-CSF-R gene (in particular the cells express GM-CSF-R gene at a lower level than the cells of human UT-7/Epo-S1 cell line (as UT-7/Epo-STI, see FIG. 9B)), or do not express GM-CSF-R gene (as clone E2, see FIG. 9B). In addition, the data show that, by using such conventional methods, the cells of the cell line of the invention express significantly high levels of Epo-R gene, in particular at a higher level than the cells of human UT-7/Epo-S1 cell line (as UT-7/Epo-STI and clone E2, see FIG. 9A).

In one embodiment, the level of expression of GM-CSF-R gene and/or Epo-R gene in human UT-7/Epo-S1 cell line is(are) detected and/or quantified using the technique used for detecting and/or quantifying the level of expression of GM-CSF-R gene and/or Epo-R gene in the cell line of the invention, and is compared to the level of expression of GM-CSF-R gene and/or Epo-R gene detected and/or quantified for the cell line of the invention.

Advantageously, in the cell line of the invention, Signal Transducer and Activator of Transcription 5 (STAT-5) is not phosphorylated or phosphorylated at a lower level than human UT7/Epo-S1 cell line, when contacted with GM-CSF. Indeed, the Inventors have shown that, after being contacted with GM-CSF, the level of phosphorylated STAT5 in the cell line of the invention is significantly low, and in particular significantly lower than in the human UT7/Epo-S1 cell line. This is consistent with the low levels of expression or absence of expression of GM-CSF-R in the cell line of the invention. Indeed, the absence of activation or the very low activation of the STAT5 pathway by GM-CSF shows that the cells of the cell line of the invention express little or no GM-CSF receptor on their surface, whereas this pathway is still activated in UT-7/Epo-S1 cell line. Interestingly, this reduction or loss of GM-CSF signaling in the cell line of the invention is a marker of a more pronounced commitment to the erythroid lineage, compared to the UT-7/Epo-S1 cell line.

The cells of the cell line of the invention preferably express high levels of the gene encoding the Receptor of B19V, i.e. Integrin-α5 (also called CD49e), in particular at a higher level than the cells of human UT-7/Epo-S1 cell line. Indeed, the data demonstrate that the cells of the cell line of the invention express the gene encoding Integrin-α5, and at a significantly higher level than the cells of human UT-7/Epo-S1 cell line (see FIG. 16).

In a preferred embodiment, the level of expression of Integrin-α5 gene is detected and/or quantified by conventional methods, such as Reverse Transcription Polymerase Chain Reaction (RT-PCR), RT quantitative PCR (RT-qPCR), western blot, and any combination thereof. Indeed, the data show that, by using such conventional methods, the cells of the cell line of the invention express significantly high levels of Integrin-α5 gene, in particular at a higher level than the cells of human UT-7/Epo-S1 cell line (as UT-7/Epo-STI and clone E2, see FIG. 16).

In one embodiment, the level of expression of Integrin-α5 gene in human UT-7/Epo-S1 cell line is detected and/or quantified using the technique used for detecting and/or quantifying the level of expression of Integrin-α5 gene in the cell line of the invention, and is compared to the level of expression of Integrin-α5 gene detected and/or quantified for the cell line of the invention.

In one embodiment, the cells of the cell line of the invention preferably express (preferably at high levels) at least one gene highly expressed in cells of the erythroid lineage in a healthy subject, in particular at a higher level than the cells of human UT-7/Epo-S1 cell line.

In one embodiment, the cells of the cell line of the invention preferably express high levels of at least one gene selected from the following group of genes:
CACHD1 (Von Willebrand Factor Type A And Cache Domain Containing 1);
EIF1AY (Eukaryotic Translation Initiation Factor 1A Y-Linked);
KDM5D (Lysine Demethylase 5D)
TXLNGY (Taxilin Gamma Pseudogene, Y-Linked);
CTSZ (Cathepsin Z)
WNT5B (Wingless-Type MMTV Integration Site Family, Member 5B);
HBE1 (Hemoglobin Subunit Epsilon 1)
NT5C3B (5′-Nucleotidase, Cytosolic IIIB);
MEST (Mesoderm Specific Transcript);
IAH1 (Isoamyl Acetate Hydrolyzing Esterase 1);
MAGEA6 (Melanoma-Associated Antigen 6);
WDR35 (WD Repeat-Containing Protein 35);
HCLS1 (Hernatopoietic Cell-Specific Lyn Substrate 1);
GAL (Galanin And GMAP Prepropeptide);
ZBTB10 (Zinc Finger And BTB Domain-Containing Protein 10);
HBZ (Hemoglobin Subunit Zeta)
PTP4A3 (Protein Tyrosine Phosphatase 4A3);
RGL3 (Ral Guanine Nucleotide Dissociation Stimulator Like 3);
MYEF2 (Myelin Expression Factor 2);
DGKH (Diglyceride Kinase Eta);

In particular at a higher level than the cells of human UT-7/Epo-S1 cell line.

Indeed, the data demonstrate that the cells of the cell line of the invention express these genes, and at a significantly higher level than the cells of human UT-7/Epo-S1 cell line (see FIG. 15).

In one embodiment, the cells of the cell line of the invention preferably express high levels of at least one gene highly expressed in cells of the erythroid lineage, in a healthy subject.

In one embodiment, the cells of the cell line of the invention preferably express low levels of at least one gene selected from the following group of genes:
ARHGEF10 (Rho Guanine Nucleotide Exchange Factor 10);
FAM105A (Family With Sequence Similarity 105, Member A);
PSD3 (Pleckstrin And Sec7 Domain Containing 3);
S1PR4 (Sphingosine-1-Phosphate Receptor 4)
ZNF711 (Zinc Finger Protein 711);
SCML2 (Sex Comb On Midleg-Like Protein 2);
CPVL (Carboxypeptidase Vitellogenic Like);
JAKMIP1 (Janus Kinase And Microtubule Interacting Protein 1);
IGFBP2 (Insulin Like Growth Factor Binding Protein 2);
ARHGAP32 (Rho GTPase Activating Protein 32);
KDM4C (Lysine Demethylase 4C);
KRT79 (Keratin 79)
KIF21A (Kinesin Family Member 21A);
MCF2 (MCF.2 Cell Line Derived Transforming Sequence);
PLS3 (Plastin 3)
CD44 (Hematopoietic Cell E- And L-Selectin Ligand, Homing Function And Indian Blood Group System);
SLC38A1 (Sodium-Coupled Neutral Amino Acid Transporter 1);
RELN (Reelin)
BTNL9 (Butyrophilin Like 9);
TDRG1 (Testis Development Related 1);

In particular at a lower level than the cells of human UT-7/Epo-S1 cell line.

Indeed, the data demonstrate that the cells of the cell line of the invention express these genes at a low level, and at a significantly lower level than the cells of human UT-7/Epo-S1 cell line, or do not express these genes (see FIG. 15).

In one embodiment, the cell line of the invention is strictly dependent on Erythropoietin (Epo) for growth. Indeed, the data demonstrate that the cells require Epo for growth.

In one embodiment, the cell line of the invention is UT7/Epo-STI (deposited under the provisions of Budapest treaty, at the Collection Nationale de Cultures de Microorganismes (CNCM, having the address: CNCM, Institut Pasteur, 25 rue du Docteur Roux, F-75724 Paris Cedex 15), on 5 Oct. 2020, under the deposit number CNCM I-5599).

The Inventors have also demonstrated for the first time a direct correlation between permissivity to B19V infection and the S/G2/M cell cycle phases, which further provides a new, additional, selection criterium for the isolation of further cell lines with high permissivity to B19. Indeed, the Inventors have shown that a majority of the cells of the cell line of the invention are in one of the S/G2/M phases of the cell cycle. Accordingly, in a preferred embodiment of the cell line of the invention, a majority of the cells are in one of the S/G2/M phases of the cell cycle.

Thus, the present invention preferably relates to a human erythroid progenitor cell line, wherein at least 90% of the cells are CD36⁺CD44⁻CD71⁺; wherein the cells:
do not express the gene encoding the Receptor of Granulocyte-macrophage colony-stimulating factor (GM-CSF-R gene), or express GM-CSF-R gene at a lower level than the cells of human UT-7/Epo-S1 cell line; and
express the gene encoding the Receptor of Erythropoietin (Epo-R gene); and
   wherein a majority (i.e. more than 50%) of the cells are in one of the S/G2/M phases of the cell cycle.

Advantageously, at least 70% of the cells are in one of the S/G2/M phases of the cell cycle, preferably at least 71% of the cells are in one of the S/G2/M phases of the cell cycle, preferably 72% cells or more, preferably 73% cells or more, preferably 74% cells or more, more preferably 75% cells or more, more preferably 76% cells or more, more preferably 77% cells or more, more preferably 78% cells or more, more preferably 79% cells or more, more preferably 80% cells or more, more preferably 81% cells or more, more preferably 82% cells or more are in one of the S/G2/M phases of the cell cycle.

In particular, the Inventors have shown that a cell cycle indicator (in particular a fluorescent cell cycle indicator), can be successfully used to discriminate cell cycle phases in human erythroid progenitor cells, and thus for selecting new cell lines with high permissivity to B19. Thus, in a preferred embodiment, the cell line according to the invention expresses at least one gene encoding a cell cycle indicator, wherein the cell cycle indicator is preferably a fluorescent cell cycle indicator, such as Fluorescent Ubiquitination Cell Cycle Indicator (FUCCI).

Accordingly, in one advantageous embodiment, the cell line of the invention further expresses at least one gene encoding a cell cycle indicator, wherein the cell cycle indicator is preferably a fluorescent cell cycle Indicator, such as Fluorescent Ubiquitination Cell Cycle Indicator (FUCCI).

At least one cell cycle indicator may be used for each of the G1, S, G2 and M phases. However, in view of the demonstration by the Inventors that highly B19 permissive cells are in one of the S/G2/M phases, it may be more advantageous to distinguish the G1 phase directly from the group of S, G2 and M phases. In this preferred embodiment, it is possible to use one or more (preferably one) indicator for the G1 phase in combination with one or more (preferably one) indicator for the S, G2 and M phases (also referred to as the S/G2/M phases). Thus, in a particularly preferred embodiment, the cell cycle indicator is a FUCCI comprising (or consisting essentially of, or consisting of) one or more (preferably one) indicator for the G1 phase in combination with one or more (preferably one) indicator for the S/G2/M phases. Thus, FUCCI preferably comprises a gene encoding Cdt1 or fragment(s) thereof (in particular amino acids 30-120 of Cdt1) and/or a gene encoding Geminin or fragment(s) thereof (in particular amino acids 1-110 or 1-60 of Geminin). In a particularly preferred embodiment, FUCCI comprises a gene encoding Cdt1 or fragment(s) thereof (in particular amino acids 30-120 of Cdt1) and a gene encoding Geminin or fragment(s) thereof (in particular amino acids 1-110 or 1-60 of Geminin). In this case, the indicator for the G1 phase is a fluorescent protein (such as Green Fluorescent Protein (GFP), Yellow Fluorescent Protein (YFP), Red Fluorescent Protein (RFP), Cyan Fluorescent Protein (CFP), mCherry, mVenus, mApple, Renilla, monomeric Kusabira-Orange 2 (mKO2), monomeric Azami-Green 1 (mAG1), mKate2, or mTurquoise, preferably mCherry) fused to CdT1 or fragments thereof (in particular amino acids 30-120 of Cdt1), and the indicator for the S/G2/M phases is a fluorescent protein fused (such as Green Fluorescent Protein (GFP), Yellow Fluorescent Protein (YFP), Red Fluorescent Protein (RFP), Cyan Fluorescent Protein (CFP), mCherry, mVenus, mApple, Renilla, monomeric Kusabira-Orange 2 (mKO2), monomeric Azami-Green 1 (mAG1), mKate2, or mTurquoise, provided that the fluorescent protein is distinct from and its fluorescence may be distinguished from that of the fluorescent protein fused to Cdt1 or Cdt1 fragment. The fluorescent protein fused to Geminin or fragment thereof ids preferably mVenus) to Geminin or fragments thereof (in particular amino acids 1-110 or 1-60 of Geminin).

In a preferred embodiment, the cell cycle phases are detected using the FUCCI system. Advantageously, in order to determine the percentage of cells in one of the S/G2/M phases, the cell cycle phases are detected using the FUCCI system. In a preferred embodiment, the FUCCI system is used/allows to detect/visualize the cell cycle phases (advantageously, in order to determine the percentage of cells in one of the S/G2/M phases). In a preferred embodiment, the cell cycle phases are visualized through the FUCCI system (advantageously, in order to determine the percentage of cells in one of the S/G2/M phases).

In a preferred embodiment, the cells of the cell line of the invention do not express the gene encoding GM-CSF-R. In a more preferred embodiment, the cell line of the invention further expresses at least one gene encoding a cell cycle indicator and do not express the gene encoding GM-CSF-R. The level of expression of GM-CSF-R gene is preferably detected by conventional methods, such as Reverse Transcription Polymerase Chain Reaction (RT-PCR), RT quantitative PCR (RT-qPCR), western blot, and any combination thereof. Indeed, the data show that, by using such conventional methods, the cells of the cell line of the invention further expressing at least one gene encoding a cell cycle indicator, do not express GM-CSF-R gene (as clone E2, see FIG. 9B).

In a preferred embodiment of the cell line of the invention further expressing at least one gene encoding a cell cycle indicator, the cell line is UT7/Epo-STI-derived clone E2 (deposited under the provisions of Budapest treaty, at the Collection Nationale de Cultures de Microorganismes (CNCM, having the address: CNCM, Institut Pasteur, 25 rue du Docteur Roux, F-75724 Paris Cedex 15), on 5 Oct. 2020, under the deposit number CNCM I-5600).

The Inventors have surprisingly demonstrated that the novel cell lines of the invention are significantly more permissive to B19 infection than the cell populations classically used for detecting and/or producing B19 (see FIG. 1B, FIG. 3 and FIG. 6A). In particular, comparative data surprisingly show that the permissivity/sensitivity of the cell lines of the invention for human parvovirus B19 infection is at least 5 times higher compared to human UT7/Epo-S1 cell line (i.e. the cell line known by the person skilled in the art to be among the cell line/population the most permissive to B19 infection). Accordingly, in a preferred embodiment, the cell line of the invention has a high permissivity/sensitivity for human parvovirus B19 infection, in particular wherein the permissivity/sensitivity of the cell line of the invention for human parvovirus B19 infection is higher than the permissivity/sensitivity of human UT7/Epo-S1 cell line for human parvovirus B19 infection. Preferably, the permissivity/sensitivity of the cell line of the invention for human parvovirus B19 infection is at least 5 times higher compared to human UT7/Epo-S1 cell line. In particularly preferred embodiments, the permissivity/sensitivity of the cell line of the invention for human parvovirus B19 infection is at least 6 times higher compared to human UT7/Epo-S1 cell line, more preferably 7 times higher, more preferably 8 times higher, more preferably 9 times higher, more preferably 10 times higher compared to human UT7/Epo-S1 cell line. In even more preferred embodiments, the permissivity/sensitivity of the cell line of the invention for human parvovirus B19 infection is at least 11 times higher compared to human UT7/Epo-S1 cell line, more preferably at least 12 times higher compared to human UT7/Epo-S1 cell line, more preferably 13 times higher, more preferably 14 times higher, more preferably 15 times higher, more preferably 16 times higher, more preferably 17 times higher, more preferably 18 times higher, more preferably 19 times higher, more preferably 20 times higher, more preferably 25 times higher, more preferably 30 times higher. Indeed, the data show that when the cell line of the invention further expresses at least one gene encoding a cell cycle indicator and does not express the gene encoding GM-CSF-R, the permissivity/sensitivity for human parvovirus B19 infection is at least 20 times higher compared to human UT7/Epo-S1 cell line (in particular more than 30 times higher for clone E2, see FIG. 6A).

The permissivity/sensitivity of the cell line for B19 may be detected and/or quantified by any appropriate technique. In particular, the permissivity/sensitivity of the cell line is detected and/or quantified at the RNA level (i.e. by detecting and/or quantifying B19 RNAs, such B19 mRNAs and/or B19 regulatory RNAs; and/or by detecting any recombinant RNA expressed by B19), preferably by RT-PCR, RT-qPCR, FISH, northern-blot, southern-blot, Nucleic Acid-Based Sensors, sequencing, and any combination thereof. In one embodiment, the permissivity/sensitivity of human UT7/Epo-S1 cell line is detected and/or quantified using the technique used for detecting and/or quantifying the permissivity/sensitivity of the cell line on the invention, and is compared to the permissivity/sensitivity detected and/or quantified for the cell line of the invention.

The cell line of the invention also provides several other advantages, of utmost importance for industrial production as well as for reliability in industrial uses. Indeed, the cell line of the invention is an immortalized cell line. In addition, the cell line of the invention is an homogeneous and stable cell line, requires low-maintenance, and gives reproducible results. In contrast, primary CD36⁺ EPC present major disadvantages for industrial uses and production, such as a high heterogeneity of the cells, the need for donors to obtain CD34⁺ cells, the technical expertise required and the cost related to cell purification and maintenance (such as expensive media and cytokines), and instability.

Thus, the cell line of the invention is preferably derived directly or indirectly from a human megakaryoblastoid cell line. The cell line of the invention is more preferably derived directly or indirectly from human UT-7 cell line (commercially available from the catalogue of Leibniz Institute (DSMZ: Deutsche Sammlung von Mikroorganismen and Zellkulturen GmbH) under number DSMZ ACC-137). The cell line is more preferably directly or indirectly derived from a human UT-7/GM cell line.

Preferably, the cell line is obtainable (or obtained, or directly obtained) by a process comprising (or consisting essentially of, or consisting of) a) growing and passaging a human erythroid progenitor cell line (preferably human UT-7 cell line (DSMZ ACC-137) or human UT-7/GM cell line), for at least 6 months in an appropriate culture medium containing Epo, and under appropriate conditions; and b) selecting/cloning one or more cell(s) that:
express the gene encoding the Receptor of Epo (Epo-R gene) and
do not express the gene encoding the Receptor of Granulocyte-macrophage colony-stimulating factor (GM-CSF-R gene) or express GM-CSF-R gene at a low level (preferably at a lower level than the cells of human UT-7/Epo-S1 cell line).

Said process may further comprise the steps of c) transfecting or transducing the cell line of step b) with a vector (e.g. a lentiviral vector) or a viral particle (e.g. a lentiviral particle) expressing one or more cell cycle indicator(s), followed by d) selecting/cloning one or more cell(s) that are in one of the S/G2/M cycle phases.

In another aspect, the invention concerns a human erythroid progenitor cell line, wherein at least 90% of the cells are CD36⁺CD44⁻CD71⁺; and wherein a majority of the cells are in one of the S/G2/M phases of the cell cycle.

The invention also concerns a human erythroid progenitor cell line, wherein at least 90% of the cells are CD36⁺CD44⁻CD71⁺; wherein a majority of the cells (e.g. more than 50%) are in one of the S/G2/M phases of the cell cycle; and wherein the cells express the gene encoding the Receptor of Epo (Epo-R gene).

Advantageously, at least 70% of the cells are in one of the S/G2/M phases of the cell cycle, preferably at least 71% of the cells are in one of the S/G2/M phases of the cell cycle, preferably 72% cells or more, preferably 73% cells or more, preferably 74% cells or more, more preferably 75% cells or more, more preferably 76% cells or more, more preferably 77% cells or more, more preferably 78% cells or more, more preferably 79% cells or more, more preferably 80% cells or more, more preferably 81% cells or more, more preferably 82% cells or more are in one of the S/G2/M phases of the cell cycle.

### Process of Generating a Cell Line Permissive for B19 Infection

Accordingly, the present invention also relates to a novel process of generating a cell line permissive for B19 infection, comprising the steps of:
a) growing and passaging a human erythroid progenitor cell line (preferably human UT-7 cell line (DSMZ ACC-137) or human UT-7/GM cell line), for at least 6 months in an appropriate culture medium containing Epo, and under appropriate conditions;
b) selecting/cloning one or more cell(s) from the cultured cell line of step a) that:
   express the gene encoding the Receptor of Epo (Epo-R gene) and
   do not express the gene encoding the Receptor of Granulocyte-macrophage colony-stimulating factor (GM-CSF-R gene) or express GM-CSF-R gene at a low level (preferably at a lower level than the cells of human UT-7/Epo-S1 cell line).

Appropriate culture medium containing Epo, and appropriate culture conditions are for example described in the section below (“Uses of the cell lines of the invention”).

In a preferred embodiment, Epo is present/added in the appropriate culture medium of step a) at a concentration ranging from 0.5 U/mL to 10 U/mL, preferably 0.7 U/mL to 9 U/mL, preferably 1 U/mL to 8 U/mL, preferably 1.2 U/mL to 7 U/mL, preferably 1.5 U/mL to 6 U/mL, preferably 1.7 U/mL to 5 U/mL, preferably 1.8 U/mL to 4 U/mL, preferably 1.9 U/mL to 3 U/mL, preferably 2 U/mL to 2.8 U/mL, preferably 2 U/mL to 2.5 U/mL, preferably 2.1 U/mL to 2.4 U/mL, preferably 2.2 U/mL to 2.3 U/mL, more preferably 2 U/mL.

In step b), one or more of the cells that express the gene encoding the Receptor of Granulocyte-macrophage colony-stimulating factor (GM-CSF-R gene) at a low level (preferably at a lower level than the cells of human UT-7/Epo-S1 cell line), or that do not express GM-CSF-R, can be selected/cloned. For this purpose, the cells of step a) are screened based on their expression level of GM-CSF-R gene. In addition, one or more of the cells that express the gene encoding the Receptor of Epo (Epo-R gene) (preferably at a high level, more preferably at a higher level than the cells of human UT-7/Epo-S1 cell line), can be selected/cloned. For this purpose, the cells of step a) are also screened based on their expression level of Epo-R gene.

The level of expression of GM-CSF-R gene and/or Epo-R gene may be detected by conventional methods, such as Reverse Transcription Polymerase Chain Reaction (RT-PCR), RT quantitative PCR (RT-qPCR), western blot, and any combination thereof.

In one embodiment, the level of expression of GM-CSF-R gene and/or Epo-R gene in human UT7/Epo-S1 cell line is detected and/or quantified using the technique used for detecting and/or quantifying the level of expression of GM-CSF-R gene and/or Epo-R gene in the cell line on the invention, and is compared to the level of expression of GM-CSF-R gene and/or Epo-R gene detected and/or quantified for the cell line of the invention.

In one embodiment, the process further comprises the steps of:
c) transfecting or transducing the cell line of step b) with a vector or a viral particle (e.g. a lentiviral particle) expressing one or more cell cycle indicator(s) (preferably under conditions allowing expression of the cell cycle indicator(s)), preferably one or more fluorescent cell cycle indicator(s), more preferably a FUCCI system); and
d) selecting/cloning one or more cell(s) from the cell line of step c) that are in one of the S/G2/M cycle phases.

The transfection or transduction (and/or the expression of the cell cycle indicator) of step c) can be either stable or transient, and may be performed using any conventional method known in the art. Those include but are not limited to lentiviral or retroviral transfer, lipofection, nucleofection, etc.

In step d), one or more of the cells that are in one of the S/G2/M cycle phases can be then selected/cloned. For this purpose, the transduced/transfected cells of step c) are then screened based on their cell cycle phase using the cell cycle indicator(s), and one or more cell(s) that is(are) in one of the S/G2/M cycle phases is(are) kept. Separation of cells in the G1 phase from cells in one of the S/G2/M cycle phases may be performed using any conventional method known in the art. In particular, when one or more fluorescent cell cycle indicator(s) is(are) used, in particular a FUCCI system, fluorescence activated cell sorting (FACS) may be used for the separation. One or more single cell(s) in one of the S/G2/M cycle phases may then or concomitantly be isolated (for instance using FACS when one or more fluorescent cell cycle indicator(s) is(are) used, in particular a FUCCI system), resulting in cloning of a new cell line permissive for B19.

In another aspect, the present invention also relates to a novel process of generating a cell line permissive for B19 infection, comprising the steps of:
a) transfecting or transducing a progenitor erythroid cell or cell line with a vector or a viral particle (e.g. a lentiviral particle) expressing one or more cell cycle indicator(s) (preferably under conditions allowing expression of the cell cycle indicator(s), preferably one or more fluorescent cell cycle indicator(s), more preferably a FUCCI system); and
b) selecting/cloning one or more cell(s) that is(are) in one of the S/G2/M cycle phases based on the cell cycle indicator.

### Uses of the Cell Lines of the Invention

The novel cell lines developed by the Inventors are highly permissive to parvovirus B19 infection. Importantly, the Inventors have shown that these cell lines allow an efficient, reliable and more sensitive B19 particle in vitro detection system, as well as a stable and efficient production of infectious B19 particles in vitro.

Accordingly, in a first aspect of uses of the cell line according to the invention, the present invention is directed to the use of the cell line according to the invention for producing parvovirus B19 in vitro, preferably infectious parvoviral B19 particles.

The use for producing parvovirus B19 in vitro preferably comprises introducing a parvovirus B19 genome into a cell line according to the invention and culturing the cell line under conditions allowing replication of parvovirus B19 genome.

The use for producing parvovirus B19 more preferably comprises infecting a cell line according to the invention with parvovirus B19, culturing the infected cell line under conditions suitable for producing B19 parvovirus; and harvesting the produced parvovirus B19. Infectious parvovirus B19 used for the infection step may be obtained in vitro from the supernatant of permissive cells infected with B19 and/or from permissive cells infected with B19 (e.g. following cell lysis). Parvovirus B19 may also be obtained from a biological sample previously taken from a B19 infected subject.

Advantageously, the use for producing parvovirus B19 comprises the steps of:
a) infecting cells of a cell line according to the invention with B19,
b) culturing said infected cells under conditions suitable for producing B19 parvovirus,
c) recovering the B19 particles produced from the culture supernatant and/or the cultured cells, and
d) optionally, purifying the recovered B19 particles.

In step a), cells of a cell line according to the invention are infected with B19 under conventional conditions for infecting permissive cells by B19. Such conditions include incubating cells of a cell line of the invention with infectious B19 (preferably using 50 to 10000 genome equivalent (ge) of B19 per cell (50-10000 ge/cell)), at a temperature ranging from 36° C. to 38° C., preferably a temperature of 37° C., for 1 h to 4 h, preferably for 1 h to 3 h, preferably for 1 h to 2 h, preferably for 1 h.

In a preferred embodiment of step a), 100 to 100000 genome equivalent (ge) of B19 is used per cell (100-100000 ge/cell), preferably 100 to 50000 ge/cell), preferably 100 to 10000 ge/cell), more preferably 100 to 9000 genome equivalent (ge) of B19 is used per cell (100-9000 ge/cell), preferably 200-8000 ge/cell, preferably 300-7000 ge/cell, preferably 400-6000 ge/cell, preferably 500-5000 ge/cell, preferably 600-4000 ge/cell, preferably 700-3000 ge/cell, preferably 800-2000 ge/cell, preferably 900-1000 ge/cell, more preferably about 500 ge/cell.

Advantageously, 10⁵ to 10⁶ cells are used (preferably at a density of 10⁶ to 10⁷ cells/mL). Advantageously, 5.10⁷ to 5.10⁸ ge of B19 are used (e.g. 500 ge/cells for 100 000 cells).

The medium used in step a) is preferably a medium essentially free of foetal calf serum and of human erythropoietin (h-Epo).

In a particular embodiment, step a) comprises the sub-steps of:
i) incubating the cell line as defined above with infectious B19 (preferably using cell and B19 quantity as defined above for step a) and medium as defined above for step a)), at a temperature ranging from 3° C. to 5° C. (preferably at a temperature of 4° C.), for 1 h to 4 h, preferably for 1 h to 3 h, preferably for 1 h to 2 h, preferably for 2 h to obtain a mixture of cells and B19;
ii) incubating the mixture of cells and B19 of step i) at a temperature ranging from 36° C. to 38° C., preferably a temperature of 37° C., for 1h to 4h, preferably for 1 h to 3 h, preferably for 1 h to 2 h, preferably for 1 h, preferably in the same medium as step i) (preferably under an atmosphere containing 5% CO₂), to obtain cells infected with B19.

The sub-step i) may be advantageously added to promote the interaction of B19 viruses with their receptors on the cell surface, as demonstrated by the Inventors.

In step b), infected cells are cultured under conditions suitable for producing B19 parvovirus. Such conditions include incubating the cells infected with B19 of step a) for at least 72 h (preferably at least 96 h), at a temperature ranging from 36° C. to 38° C., preferably a temperature of 37° C. (preferably under an atmosphere containing 5% CO₂). The medium used in step b) is preferably a complete medium. For example a complete medium comprises, or consists essentially of, or consists of, the following components: alpha minimum essential medium (αMEM) supplemented with 10% foetal calf serum (FCS), L-glutamine (e.g. at a concentration of 2 mM), penicillin (e.g. at a concentration of 100 U/mL), streptomycin (e.g. at a concentration of 100 μg/mL), and recombinant human Erythropoietin (e.g. at a concentration of 2 U/mL).

In a preferred embodiment, Epo is present/added in the medium of step b) at a concentration ranging from 0.5 U/mL to 10 U/mL, preferably 0.7 U/mL to 9 U/mL, preferably 1 U/mL to 8 U/mL, preferably 1.2 U/mL to 7 U/mL, preferably 1.5 U/mL to 6 U/mL, preferably 1.7 U/mL to 5 U/mL, preferably 1.8 U/mL to 4 U/mL, preferably 1.9 U/mL to 3 U/mL, preferably 2 U/mL to 2.8 U/mL, preferably 2 U/mL to 2.5 U/mL, preferably 2.1 U/mL to 2.4 U/mL, preferably 2.2 U/mL to 2.3 U/mL, more preferably 2 U/mL.

Step b) of culturing the infected cells under conditions suitable for producing B19 parvovirus may comprise the use of chloroquine (e.g. chloroquine is added to the culture medium). Chloroquine is a compound having the chemical formula C₁₈H₂₆ClN₃ and the IUPAC (International Union of Pure and Applied Chemistry) name (RS)-N-(7-chloroquinolin-4-yl)-N,N-diéthyl-pentane-1,4-diamine. Chloroquine is advantageously added to the culture medium to boost virus entry and prevent the degradation of incoming viruses through a blockade of lysosome transfer. In one embodiment, chloroquine is added to the medium of step b) (preferably to a final concentration ranging from 10 to 50 μM, preferably 15 to 45 μM, preferably 20 to 40 μM, preferably 25 to 35 μM, preferably 20 to 30 μM, more preferably 25 μM of chloroquine).

In step c), recovering the B19 particles produced from the cultured cells may comprise collection of supernatant and/or lysis of the recovered cultured cell, when the recovery of B19 is made at least partially from cultured cells. The lysis may be partial or total and may be performed using any conventional method known in the art, including chemical lysis (e.g. osmotic shock, enzymatic or detergent lysis), or physical lysis (e.g. ultrasounds lysis, freeze-thaw lysis or homogenization lysis).

In optional step d), the recovered B19 particles are purified using conventional methods. Such methods include precipitation, centrifugation, ultracentrifugation, chromatography, gradient, and any combination thereof.

Advantageously, the produced parvovirus B19 is infectious (infectious parvoviral B19 particles are thus obtained).

The produced parvovirus B19 may be a native parvovirus B19 or a recombinant parvovirus B19.

In another aspect of uses of the cell line according to the invention, the present invention also relates to the use of the cell line according to the invention for detecting parvovirus B19 (preferably infectious parvoviral B19 particles) in a biological sample in vitro. Advantageously, the use for detecting parvovirus B19 in vitro preferably comprises contacting the cell line according to the invention with a biological sample susceptible to comprise parvovirus B19 and culturing the cell line under conditions allowing replication of parvovirus B19 genome.

The use for detecting parvovirus B19 more preferably contacting the cell line according to the invention with a biological sample susceptible to comprise parvovirus B19, culturing the cell line under conditions suitable for producing and/or detecting B19 parvovirus; and optionally harvesting the produced parvovirus B19.

Advantageously, the use for detecting parvovirus B19 comprises the steps of:
a) contacting the cell line according to the invention with a biological sample susceptible to comprise parvovirus B19,
b) culturing said infected cells under conditions suitable for producing B19 parvovirus,
c) detecting B19 parvovirus (preferably B19 particles) produced in the culture supernatant and/or the cultured cells of step b).

In a preferred embodiment, steps a) and b) of the use for detecting parvovirus B19 are as steps a) and b), respectively, for producing parvovirus B19 (as defined above), except that, in step b) of the use for detecting parvovirus B19, the cells are incubated (cultured) for at least 48 h.

Step c) may comprise an optional step of recovering the produced parvovirus B19, performed before detection. In a preferred embodiment, this optional step of the use for detecting parvovirus B19 is as step c) for producing parvovirus B19 (as defined above),

In step c), the B19 particles may be detected at the nucleic acid level, at the protein level, at the particle level, and any combination thereof.

The preferred techniques that can be used for detecting B19 at the nucleic acid level (e.g. B19 genome, B19 cDNA, B19 transcripts (e.g. B19 mRNA and/or regulatory RNA), DNA, cDNA and transcripts encoding recombinant proteins inserted in B19 genome, and any combination thereof) include PCR, qPCR, RT-PCR, RT-qPCR, FISH, northern-blot, southern-blot, Nucleic Acid-Based Sensors, sequencing, and any combination thereof.

The preferred techniques that can be used for detecting B19 at the protein level (e.g. B19V proteins and/or recombinant proteins inserted in B19 genome) include FACS, immunostaining, immunohistochemistry, western-blot, dot-blot, mass spectrometry, chromatography, ELISA, and any combination thereof.

The preferred techniques that can be used for detecting B19 at the particle level include Immunostaining, immunohistochemistry, PCR after DNAse treatment, Nucleic Acid-Based Sensors, mass spectrometry, infection test, ELISA, and any combination thereof.

It is possible to quantify parvovirus B19 in a biological sample, using the cell line of the invention. Thus, in one embodiment, the use for detecting parvovirus B19 is for quantifying parvovirus B19 (preferably infectious parvoviral B19 particles) in vitro in a biological sample, in particular for the purpose of evaluating the efficiency of a viral reduction process on parvovirus B19 and/or for diagnosing a parvovirus B19 infection in a subject. The above-mentioned techniques may also be used for quantifying parvovirus B19.

To quantify B19 in a sample, it may be advantageous to calculate/determine the Tissue Culture Infectious Dose 50 (TCID50). TCID50 is a measure of infectious virus titre. This endpoint dilution assay quantifies the amount of B19 virus required to infect 50% of the host cells. To quantify B19 in a sample, cells of the cell lines of the invention are for example plated and serial dilutions of the sample (the sample may be e.g. B19 parvovirus produced in the culture supernatant and/or the cultured cells, as in step b) described above) are added. After incubation, the percentage of infected cells is determined/quantified.

Thus, in one embodiment, step c) of detecting B19 parvovirus (preferably B19 particles) produced in the culture supernatant and/or the cultured cells of step b) further comprises calculating/determining the Tissue Culture Infectious Dose 50 (TCID50), especially when B19 quantification is desired.

The present invention thus also relates to the use of the cell line according to the invention for quantifying parvovirus B19 (preferably infectious parvoviral B19 particles) in vitro in a biological sample, in particular for evaluating the efficiency of a viral reduction process on parvovirus B19 and/or for diagnosing a parvovirus B19 infection in a subject.

The preferred techniques that can be used for quantifying B19 at the nucleic acid level (e.g. B19 genome, B19 cDNA, B19 transcripts (e.g. B19 mRNA and/or regulatory RNA)) include PCR, qPCR, RT-PCR, RT-qPCR, FISH, northern-blot, southern-blot, Nucleic Acid-Based Sensors, sequencing, and any combination thereof.

The preferred techniques that can be used for quantifying B19 at the protein level include FACS, immunostaining, immunohistochemistry, western-blot, dot-blot, mass spectrometry, chromatography, ELISA, and any combination thereof.

The preferred techniques that can be used for quantifying B19 at the particle level include Immunostaining, immunohistochemistry, PCR after DNAse treatment, Nucleic Acid-Based Sensors, mass spectrometry, infection test, ELISA, and any combination thereof.

The present invention also relates to a use of the cell line according to the invention for evaluating in vitro the efficiency of a viral reduction process (such as a viral elimination step, a viral inactivation step, or any combination of one or more viral elimination step(s) and one or more viral inactivation step(s)) on parvovirus B19.

Evaluating in vitro the efficiency of a viral reduction process on parvovirus B19 may comprise quantifying B19 in a biological sample taken before and after one or more step(s) of viral reduction (including one or more viral elimination step(s) and/or one or more viral inactivation step(s)), e.g. in order to confirm/verify that B19 quantity is lower after the step(s) of viral reduction than before the step(s) of viral reduction, and even preferably to assess the level of parvovirus B19 reduction obtained using the viral reduction process. Such level of reduction is generally expressed in decimal logarithm (log 10 or log). The use for evaluating the efficiency of a viral reduction process on parvovirus B19 may notably include checking whether or not the viral reduction process permits to reduce B19 levels by at least 4 logs, since such reduction level is often required by health authorities for processes of preparation of blood-derived products.

The present invention also relates to the use of the cell line according to the invention for detecting/diagnosing in vitro a parvovirus B19 infection in a subject of interest from a biological sample of said subject. In this case, B19 is detected in a biological sample of the subject to be diagnosed, using the cell line of the invention.

Diagnosing in vitro a parvovirus B19 infection in a subject of interest may comprise detecting B19 in a biological sample of the subject, using the cell line of the invention. In this case, if B19 is detected, the subject of interest is diagnosed as being infected with B19. In one embodiment, B19 is also detected in a biological sample of a reference subject, used as a positive or negative control, using the cell line of the invention. It may also be useful to quantify B19 in the biological sample of the subject of interest and/or in the biological sample of the reference subject. Thus, in one embodiment, diagnosing a parvovirus B19 infection in a subject comprises quantifying B19 in a biological sample of the subject of interest, using the cell line of the invention. In such case, it may be useful to quantify B19 in the biological sample of the reference subject and compare the level of parvovirus B19 quantified in the biological sample of the subject of interest with the level of parvovirus B19 quantified in the biological sample of the reference subject.

In one embodiment, the use of the cell line according to the invention for detecting/diagnosing in vitro a parvovirus B19 infection in a subject of interest comprises, consists essentially of, or consists of, the following steps:
a) detecting parvovirus B19 in a biological sample from the subject of interest, using the cell line of the invention;
b) detecting parvovirus B19 in a biological sample from a reference subject, using the cell line of the invention;
c) comparing the level of parvovirus B19 quantified in step a) with the level of parvovirus B19 quantified in step b);
d) diagnosing a B19 infection in a subject of interest, preferably wherein the subject is diagnosed as being infected with B19 if the level of parvovirus B19 quantified in step a) is higher than the level of parvovirus B19 quantified in step b) and if the reference subject is a healthy subject.

The present invention also relates to the use of the cell line according to the invention, for in vitro screening of compounds/active agents for parvovirus B19 antiviral activity/effect. In this case, B19 production is quantified in the cell line of the invention cultivated in the absence (negative control sample) or presence (test sample) of multiple compounds/active agents to be screened, and preferably also in the cell line of the invention cultivated in the presence of a compound known as having anti-parvovirus B19 activity/effect (positive control). Any type of compound/active agent may be screened.

The present invention also relates to the use of the cell line according to the invention, for evaluating in vitro the parvovirus B19 antiviral activity/effect of a compound/active agent. This use is similar to the screening use, except that only one compound/active agent is to be evaluated.

In another aspect of uses of the cell line according to the invention, the present invention also relates to the use of the cell line according to the invention for the detection and evaluation of any pathogen with erythroid tropism.

### Methods of Using the Cell Lines of the Invention

The novel cell lines developed by the Inventors are highly permissive to parvovirus B19 infection. Importantly, the Inventors have shown that these cell lines allow an efficient, reliable and more sensitive B19 particle detection system, as well as a stable and efficient production of infectious B19 particles in vitro.

Accordingly, the present invention is directed to a method for producing parvovirus B19 in vitro, preferably infectious parvoviral B19 particles using the cell line according to the invention.

The present invention also relates to a method for detecting parvovirus B19 in vitro in a biological sample using the cell line according to the invention.

The present invention also relates to a method for quantifying parvovirus B19 (preferably infectious parvoviral B19 particles) in vitro using the cell line according to the invention, in particular for evaluating in vitro the efficiency of a viral reduction process on parvovirus B19 and/or for diagnosing a parvovirus B19 infection in a subject in vitro from a biological sample of said subject.

The present invention also relates to a method for evaluating in vitro the efficiency of a viral reduction process on parvovirus B19, using the cell line according to the invention.

The present invention also relates to a method for detecting/diagnosing a parvovirus B19 infection in a subject in vitro from a biological sample of said subject, using the cell line according to the invention.

The present invention also relates to a method for screening in vitro compounds/active agents for parvovirus B19 antiviral activity/effect, using the cell line according to the invention.

The present invention also relates to a method for evaluating in vitro the parvovirus B19 antiviral activity/effect of a compound/active agent, using the cell line according to the invention.

Any more precise embodiment described above with respect to uses of the cell line according to the invention (see the section “Uses of the cell lines of the invention”) is also contemplated for the above-described methods.

### DESCRIPTION OF THE FIGURES

FIG. 1. Comparison of the B19V sensitivity and permissiveness of hematopoietic cell lines. (A) B19V transcription profile (adapted from reference 23). The major transcription unit of the B19V duplex genome (GenBank accession no. AY386330) is shown to scale at the top, with the P6 promoter, 2 splice donors (D1, D2) and 4 acceptors (Al to A4) sites. In gray, mRNA encoding the VP2 viral proteins, with nucleotides (nts). At the bottom, the primers and probe used for the RT-PCR amplification of VP2. (B) Bone marrow-derived primary Erythroid Progenitor Cells (CD36⁺ EPCs), human leukemic cell lines (TF1, TF1-ER, UT7/Epo, UT7/Epo-STI) and isolated clones (KU812Ep6, UT7/Epo-cl3 and UT7/Epo-S1) were seeded in triplicate and inoculated with or without B19V in culture medium supplemented with Epo (2 U/mL)(/Epo), or granulocyte macrophage colony-stimulating factor (GM-CSF) (2.5 ng/mL)(/GM) for TF1 and TF1-ER. When specified, cells were cultivated with (+) or without (−) Chloroquine (CQ, 25 μM). No CQ treatment was applied to CD36+EPC. 72 h post-infection, cells were pelleted and lysed, RNA was extracted and analyzed by RT-qPCR for VP2 to quantify B19 viral genome expression, and for (3-actin for cell number normalization. For each cell line, the results without B19V correspond to the negative control. Relative B19V threshold cycle (Ct) values were normalized relative to b-actin Ct and expressed according to the 2^{-□□Ct} a method with normalization against mean VP2 expression for UT7/Epo-S1 cells without CQ (n=6). Results are presented as means±SEM of 3 independent experiments. *p<0.05; ** p<0.01; *** p<0.001; NS=No Significance. ND=Not Detected.

FIG. 2. Comparison of B19V sensitivity of hematopoietic cell lines. (A) Cell viability was assessed 72 h post-infection. The results shown are the means+SD of three independent experiments. (B) UT7/Epo-STI cells and CD36+EPCs were cultured in triplicate, with or without B19V, for 72 h. At 24, 48 and 72 h post-inoculation, cells were collected by centrifugation. RNA was extracted from the cell pellet and VP2 mRNA levels were analyzed to quantify B19 viral DNA expression, and β-actin mRNA levels were analyzed for cell number normalization. For each cell line, results without B19V correspond to the negative control. Relative B19V threshold cycle (Ct) values were normalized relatively to the β-actin Ct (log B19V/actin). The results shown are the means±SEM of three independent experiments. *p<0.05; ** p<0.01; ***p<0.001; NS=No Significance.

FIG. 3. B19V-sensitivity of UT7/Epo-STI cells is linked to maturation stage. UT7/Epo-STI cells were cultured for 48 h before inoculation with B19V, without (−) or with JQ1 (0.5 μM) or TGF-β (2 ng/mL). 72 h post-inoculation, relative levels of B19V VP-2 mRNA were evaluated with UT7/Epo-S1 cells as the reference.

FIG. 4. Generation of a UT7/Epo-STI cell line with stable expression of the Fluorescence Ubiquitination Cell Cycle Indicator (FUCCI). (A) Experimental design for the generation of the UT7/Epo-FUCCI cell line. Bottom: Two-color cell cycle mapping with the FUCCI2a Cell Cycle Sensor and right, flow cytometry analysis of exponentially growing UT7/Epo-STI and UT7/Epo-FUCCI cells. The profile shown corresponds to one representative experiment. (B) DNA content and FUCCI profiles for the same sample. Exponentially growing UT7/Epo-FUCCI cells were stained with Hoechst 33342. DNA content (Hoechst on the x-axis; cell count on the y-axis) and FUCCI proteins (m-Venus on the x-axis; m-Cherry on the y-axis) were concomitantly evaluated by flow cytometry. Bottom: Overlay of gated cell cycle populations, as determined by FUCCI analysis with DNA content profile.

FIG. 5: Cell cycle profile of the exponentially growing UT7/Epo-FUCCI cell line and clones, as determined by flow cytometry (m-Venus on the x-axis; m-Cherry on the y-axis). The profile shown corresponds to one representative experiment from four performed.

FIG. 6. Improvement of B19V sensitivity and permissiveness according to cell cycle status. (A) UT7/Epo-S1 cells (51), UT7/Epo-STI cells expressing the FUCCI system (FUCCI) and 11 UT7/FUCCI-derived isolated clones were inoculated with B19V. Relative levels of B19V mRNA were determined 72h post-infection, with UT7/Epo-S1 as the reference, and cell lines were classified based on B19V sensitivity as group I for 51-equivalent clones, group II for FUCCI-equivalent clones, and group III for highly permissive clones. The results shown are the means±SD of 3 independent experiments for groups I and II, and n=9 for group III clones. (B) The cell cycle status of exponentially growing FUCCI cell lines and isolated clones was assessed by flow cytometry. The results shown are the means±SEM of three independent measurements.

FIG. 7. Comparison of relative levels of B19 mRNA (as in FIG. 6A) and cell cycle status (as in FIG. 6B). Each dot corresponds to the mean result for a single cell line or clone (n=3), classified to groups I (white marks ∘), II (grey marks [symbol] ) and III (black marks ●). Logarithmic regression analysis and R² values are presented.

FIG. 8. Comparison of relative levels of B19 mRNA and percentage of cells in respective cell cycle status. Percentage of cells in: A) early-G1 phase (e-G1), B) G1 phase, C) transition from G1 to S phase (G1/S). Each dot corresponds to the mean result for a single cell line or clone (n=3) assigned to groups I (white marks ∘), II (grey marks [symbol] ) and III (black marks ●). A logarithmic transformation of the linear regression analysis and R² values are presented.

FIG. 9. Response to cytokines. Expression of Receptor for A) Erythropoietin (Epo-R) and B) GM-CSF (GM-CSF-R) mRNA was evaluated by RT-qPCR. The results shown are the means±SEM of three independent experiments performed with triplicates. C) Starved Cells (UT7/Epo-S1, UT7/Epo-STI, UT7/Epo-FUCCI) were stimulated with Epo (E: 10U/mL), GM-CSF (GM: 25ng/mL) or TPO (100 ng/mL) or left unstimulated (−). After lysis, cell extracts were analyzed by western-blot using antibodies raised against total (α-STAT-5, Cell Signaling Technology cat. No 94205) or phosphorylated forms of STAT-5 (α-pSTAT-5, Cell Signaling Technology cat. No 9351), and B23 for cell extract normalization (α-B23, Santa Cruz Biotechnology cat. No 271737).

FIG. 10. Proliferation of UT-7 cell lines. Cells (1.10⁵/mL) were grown in culture media containing Epo (2 U/mL). During 7 days, cell proliferation is daily assessed by counting live cells with an hemacytometer after a Trypan Blue staining. Results are means±SE of 6 independent experiments. Proliferation of cell lines are compared in graph A (UT7/Epo-S1 versus UT7/Epo-STI), B (UT7/Epo-E2 versus UT7/Epo-STI) and C (UT7/Epo-S1 versus UT7/Epo-E2)

FIG. 11. Permissivity to B19 infection of UT-7 cell lines. A) Schematic representation of the protocol used in B. B) Evaluation of B19 genome transcription (mRNA, left) and replication (DNA, right). After inoculation, cells were grown 72 hpi in culture media with (+) or without (−) chloroquine (CQ). Data are expressed as means±SD of 6 independent experiments.

FIG. 12. Production of B19 genome copies equivalent (Geq) per mL of cell culture. Cells (day 8 CD36⁺EPC, UT7/Epo-S1, UT7/Epo-STI and UT7/Epo-E2) were inoculated with B19. 24 h after inoculation (24 hpi), cells were washed. 3 days later (96 hpi), supernatant was collected, DNA was isolated and B19 DNA was quantified by qPCR according to a B19 genome DNA standard (GenBank accession no. AY386330). Results are means±SD of 3 independent experiments. For CD36⁺EPC, results are means±SD of three distinct day-8 erythroid culture from CD34⁺ hematopoietic stem cells isolated from 3 different umbilical cord blood.

FIG. 13. Permissivity to B19V infection of normal erythroid progenitor cells (CD36⁺EPC) and UT-7 cell lines. Bone marrow-derived primary Erythroid Progenitor Cells (CD36⁺EPCs) and UT-7/Epo cells lines (UT7/Epo-S1, STI and E2) were seeded in triplicate and inoculated with or without B19V in culture medium. When specified, cells were cultivated with (+) or without (−) Chloroquine (25 μM). 96 h post-infection, cells were pelleted and lysed, RNA was extracted and analyzed by RT-qPCR for VP2 to quantify B19 viral genome expression, and for β-actin for cell number normalization. For each cell line, the results without B19V correspond to the negative control. Relative B19V threshold cycle (Ct) values were normalized relative to β-actin Ct and expressed according to the 2^{-ΔΔCt} method. Data are expressed as means±SD of 6 independent experiments. B) Graph presenting data without chloroquine for magnification of the Y-axis scale.

FIG. 14. RNA sequencing of UT-7 cell lines (UT-7/Epo-S1, UT-7/Epo-STI cell line and derived clones (E2, G7 and H11). Low dimensional embedding (PCA: Principal Component Analysis) of all samples. N=3 for each cell line.

FIG. 15. Top 40 differentially expressed genes. Differentially Expressed Sequences (DESeq) between UT-7/Epo-S1 (S1, n=3) and UT-7/Epo-E2 (E2, n=3) cell lines. A) Top 20 up-regulated genes in UT7/Epo-E2 cell line versus UT7/Epo-S1. B) Top 20 down-regulated genes in UT7/Epo-E2 cell line versus UT7/Epo-S1. Each column corresponds to one sample, listed below the figure.

FIG. 16. Analysis of B19V receptor in UT-7 cell lines, Integrin-α5 (CD49e).

FIG. 17. Correlation of Integrin-α5 (CD49e) expression with cell cycle phases.

FIG. 18. Correlation between Integrin-α5 (CD4e) expression and permissivity to B19V. Without Chloroquine.

FIG. 19. Correlation between Integrin-α5 (CD49e) expression and permissivity to B19V. With Chloroquine.

### EXAMPLES

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

### 1. Example 1: New Human Erythroid Progenitor Cell Lines With Enhanced Permissivity to B19 Parvovirus Infection

### 1.1. Materials and Methods

### 1.1.1. Cell Lines

Three distinct UT-7/Epo cell lines were used: 1) UT-7/Epo-S1, a subclone of UT-7/Epo (16), was obtained from Dr Kazuo Sugamura (Tohoku University Graduate School of Medicine, Japan). 2) UT-7/Epo and UT7/Epo-Cl3, a subclone isolated from UT-7/Epo3) UT7/Epo-STI cells were derived from UT-7/GM cell line and were maintained at low passage, with stringency for erythroid features. UT-7 cells were maintained at 37° C., under an atmosphere containing 5% CO2, in alpha minimum essential medium (αMEM) supplemented with 10% fetal calf serum (FCS), 2 mM L-glutamine (Hyclone), 100 U/mL penicillin, 100 μg/mL streptomycin and 2 U/mL recombinant human Erythropoietin (rh-Epo, Euromedex, RC213-15). Where specified, 0.5 μM JQ1 (Sigma-Aldrich, France) or 2 ng/mL TGF-β (Peprotech, France) was added to the culture medium for two days before B19V infection. TF1 (12) and TF1-ER erythroleukemia cells were maintained in Roswell Park Memorial Institute (RPM!) 1640 medium supplemented with 10% FCS, 2 mM L-glutamine (Hyclone), 100 U/mL penicillin, 100 μg/mL streptomycin and 2 U/mL rh-Epo or 5 ng/mL human granulocyte macrophage colony-stimulating factor (GM-CSF, Peprotech).

KU812Ep6 cells (15), were maintained in RPMI-1640, 2 U/mL rh-Epo, 10% FCS, 100 U/mL penicillin, 100 μg/mL streptomycin and Insulin Transferrin Selenium-X supplement (ITS-X, Gibco), at 37° C., 5% CO2. Human embryonic kidney (HEK) 293T and NIH-3T3 cells were maintained in Dulbecco's modified Eagle's medium (DMEM) supplemented with 10% FCS, 2 mM L-glutamine, 100 U/mL penicillin and 100 μg/mL streptomycin.

### 1.1.2. CD36⁺ Erythroid Progenitor Cell (EPC) Line Generation

Umbilical cord blood (CB) units from normal full-term deliveries were obtained, with the informed consent of the mothers, from the Obstetrics Unit of Saint Louis Hospital, Paris, and collected in placental blood collection bags (Maco Pharma, Tourcoing, France).

Blood mononuclear cells were purified by Ficoll density gradient separation (Leucosep, Greiner Bio-one) and Hanks medium (Thermo-Fisher). Low-density cells were recovered and enriched for CD34⁺ cells by automated cell sorting (CD34 isolation kit and autoMACS System, Miltenyi Biotec). CD34⁺ cells were cultured in serum-free expansion medium: IMDM, 15% BIT 9500 (Stem Cell Technologies), 60 ng/mL rh-stem cell factor (SCF), 10 ng/mL rh-IL-3, 10 ng/mL rh-IL-6, 2 U/mL rh-Epo, 100 U/mL penicillin and 100 μg/mL streptomycin. After seven days of culture, CD36⁺ cells were isolated with biotin-coupled anti-CD36 antibody and anti-biotin microbeads on an autoMACS System. CD36⁺ EPCs were obtained by lentivirus-mediated transduction with the hTERT and E6/E7 genes from human papillomavirus type 16, as previously described, and were grown in expansion medium to generate a continuous CD36⁺ EPC line.

### 1.1.3. B19 Virus Stock and Cell Inoculation

Plasma samples containing high titers of infectious B19V from asymptomatic blood donors were provided by the Etablissement Français du Sang (EFS). Plasma samples were determined to be negative for both B19V IgG and IgM, with a viral titer of 10¹¹ B19V DNA genome equivalent (ge)/mL. Briefly, cells were maintained in exponential growth condition by dilution to 0.3×10⁶ cells/mL the day before infection. On the day of infection, cells were washed and diluted in FCS-free medium without Epo, at a density of 10·10⁶ cells/mL. B19V inoculation was carried out in a 96-well plate, with 10 μL of cell suspension (10⁵ cells) and 50 μL of a 100-fold dilution of B19V plasma (10⁹ ge/mL), corresponding to a mean of 500 ge/cell. The cells were then incubated at 4° C. for 2 h, and then at 37° C., for 1 h, under an atmosphere containing 5% CO2. We added 140 μL of complete medium and maintained the cells in culture until 72 h. Where specified, we added chloroquine (CQ) to the complete medium, at a final concentration of 25 μM. Cell viability was assessed by trypan blue exclusion test (0.4% in PBS, Thermo Fisher Scientific), by counting blue and total cells under a microscope, with a hemocytometer. After correction for the dilution factor, viability was calculated as follows: % viable cells=[1−(number of blue cells/number of total cell)]×100. At 24, 48 or 72 h post infection (hpi), cells were centrifuged (8 mins at 300×g), supernatants were discarded and cell pellets were frozen at −80° C. until analysis.

### 1.1.4. Detection of B19V: RNA Extraction and Duplex RT-qPCR

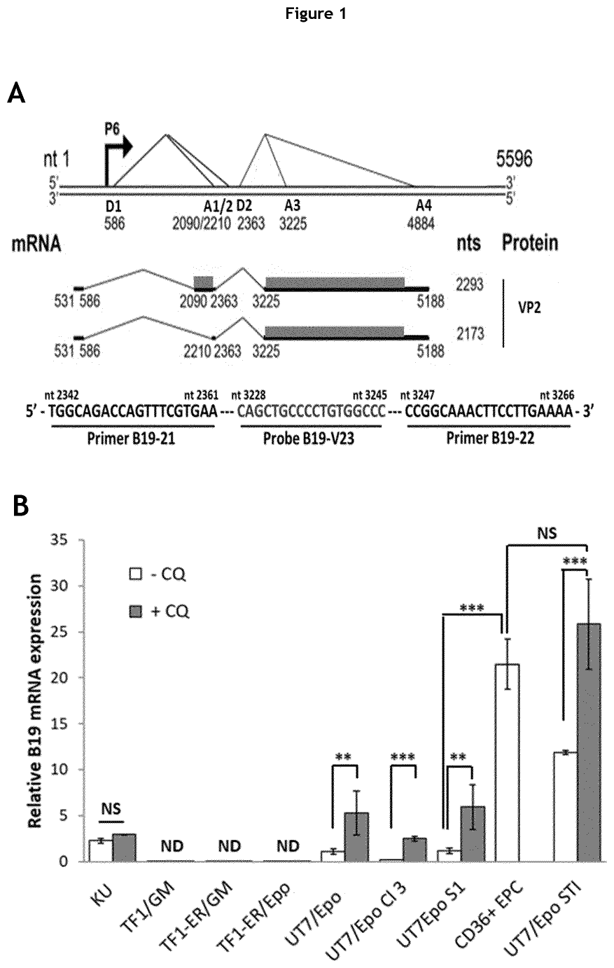

Total RNA was extracted from cell pellets with the RNeasy 96 QIAcubeHT kit and a QIAcubeHT machine, according to the manufacturer's instructions. The extraction step included DNase treatment for 15 minutes, to decrease the risk of genomic DNA amplification during PCR. Real-time reverse transcription-quantitative PCR (RT-qPCR) was performed with the Taqman Fast Virus one-step PCR kit (Applied Biosystems). B19 VP2 transcripts were amplified with the sense primer B19-21 5′-TGGCAGACCAGTTTCGTGAA-3′ (nts 2342-2361; SEQ ID NO:1), the antisense primer B19-22 5′-CCGGCAAACTTCCTTGAAAA-3′ (nts 3247-3266; SEQ ID NO:2) and the probe B19-V23 5′-VIC-CAGCTGCCCCTGTGGCCC-3′ (nts 3228-3245; SEQ ID NO:3). For control and normalization with respect to the number of cells, we used a duplex strategy. A target sequence of the spliced beta actin transcript was selected and amplified with the sense primer actin-S 5′-GGCACCCAGCACAATGAAG-3′ (SEQ ID NO:4), the antisense primer actin-AS 5′GCCGATCCACACGGAGTACT-3′ (SEQ ID NO:5) and the probe actin-FAM 5′ -FAM-TCAAGATCATTGCTCCTCCTGAGCGC-3′ (SEQ ID NO:6). Reactions were performed on 5 μL of extracted RNA with the QuantStudio 3 PCR system and. The reaction began with reverse transcription at 48° C. for 15 mins, followed by inactivation of the reverse transcriptase and activation of the polymerase by heating at 95° C. for 10 mins, followed by 40 cycles of 15s at 95° C. and 30 s at 60° C. The PCR program was optimized for amplification of the VP2 spliced transcripts rather than the VP2 genomic sequence (FIG. 1A).

### 1.1.5. Fucci2a Lentivirus Production and Cell Transduction

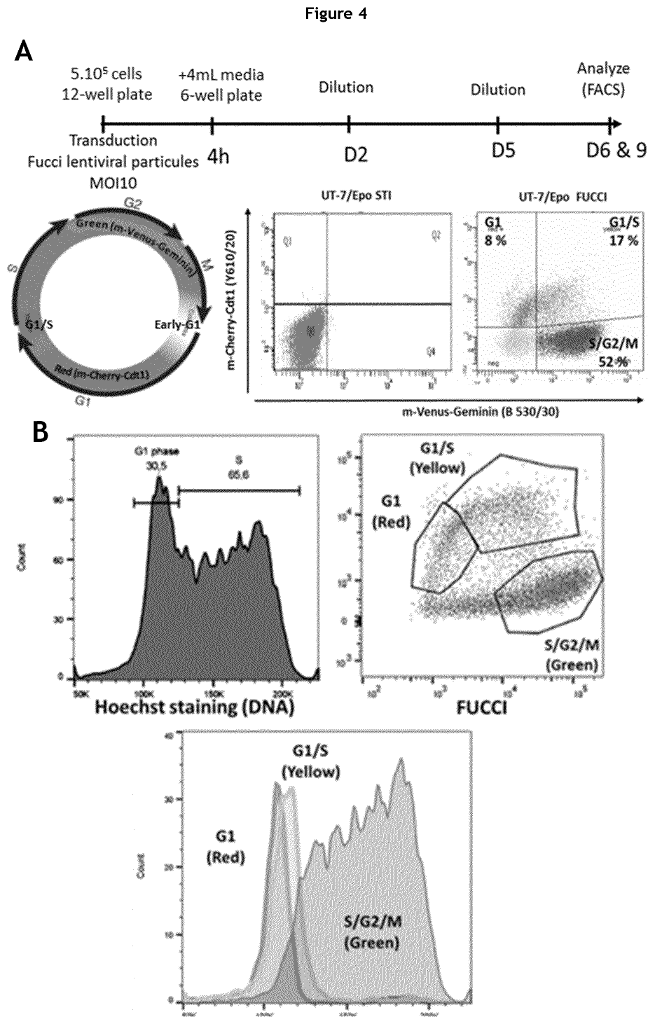

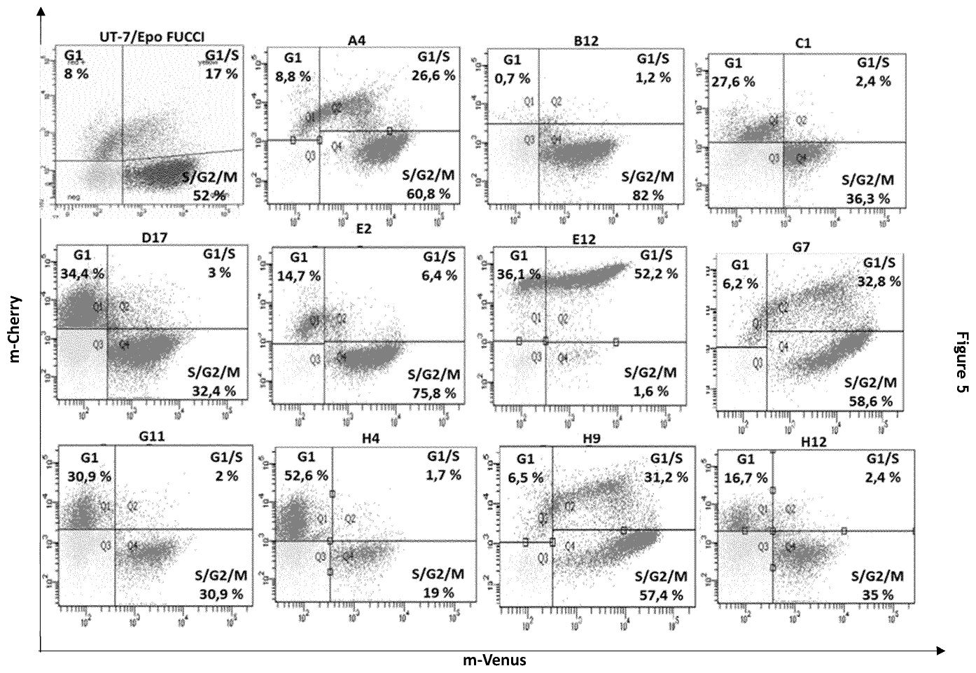

The Fucci2a DNA sequence (RDB13080, RIKEN BioSource Center; SEQ ID NO:7; resulting protein sequence shown in SEQ ID NO:8) was synthetized into the LTGCPU7 lentiviral vector backbone without the puromycin resistance-gene cassette, and under the control of the EF1α promoter and enhancer. Lentiviral particles were produced by the transient transfection of HEK293T cells with the five-plasmid packaging system, by PElpro (Polyplus transfection). These particles were then concentrated by ultracentrifugation. Infectious titres were determined in NIH-3T3 cells. We transduced 0.5×10⁶ UT7/Epo-STI cells with FUCCI particles at a mean of infection of 10, in 200 μL of complete medium, and the cells were kept at 37° C. for 4 h. Cells were subsequently diluted at 0.1×10⁶ cells/mL. On days 6 and 9 post-transduction, cells were analysed by cytometry for the expression of FUCCI proteins (FIG. 4A).

### 1.1.6. FACS Analysis of Cell Cycle Status

Fucci2a bicistronic expression was monitored with an LSRFortessa cytometer (BD Biosciences).

Fluorescent fusion proteins were detected with the 488 nm blue laser and a 530/30 nm bandpass filter (B530/30) for mVenus-hGeminin, and the 590 nm yellow laser and a 610/20 nm bandpass filter (Y610/20) for mCherry-hCdt1 (FIG. 4A). For alternative monitoring of the cell cycle according to DNA content, cells were stained with the permeable DNA dye Hoechst 3342 (10 μg/mL) for 1 h at 37° C., and immediately analysed for DNA content with the 355 nm violet laser and a 450/40 nm bandpass filter (V450/40).

FACSDiVa and FlowJo X software (BDBiosciences) were used to operate the instrument and for data analysis, respectively.

### 1.1.7. UT7/Epo-FUCCI Clone Generation

UT7/Epo-FUCCI refers further to a UT7/Epo-STI pool expressing FUCCI. UT7/Epo-FUCCI clones were isolated in a U-bottom 96-well plate, by limiting dilution, with one seeded cell per well in 100 μL of complete medium. Cells were visualized by microscopy, and wells containing more than one cell, or non-fluorescent cells were excluded. Clones were then separately expanded with an assigned name corresponding to their location on the plate. After expansion, each clone was considered further as a new cell line. A cell bank of 156 isolated clones was constituted (stored at −80° C. in 90% FCS, 10% DMSO) and isolated clones were subjected to FUCCI expression profiling. The stability of the cell cycle profiles of the isolated clones was controlled both sequentially, for at least five independent cultures, and for 10 passages of the same culture.

### 1.2. Results

To assess and compare the degree of permissiveness to B19V, hematopoietic cell lines were infected with B19V and maintained for 72h. Where specified, chloroquine (CQ) was added to boost virus entry and prevent the degradation of incoming viruses through a blockade of lysosome transfer. Active transcription of the B19V genome in host cells was evaluated by RT-qPCR for the VP2 capsid gene (FIG. 1A), with normalization to beta-actin gene expression. As a reference to calculate relative B19 mRNA expression, the value for UT7/Epo-S1 without chloroquine was set to 1 (FIG. 1B). As previously reported, UT7/Epo-S1 and KU812Ep6 cells were less permissive to B19V than CD36⁺ EPCs.

Chloroquine treatment markedly enhanced UT7/Epo-S1 sensitivity (5-fold), but without reaching the level obtained for CD36⁺ EPCs. VP2 expression was undetectable in both the parental TF1 erythroleukemia cell line and a TF1-ER cell line expressing a full Epo-receptor, under the control of GM-CSF or Epo, with or without chloroquine treatment.

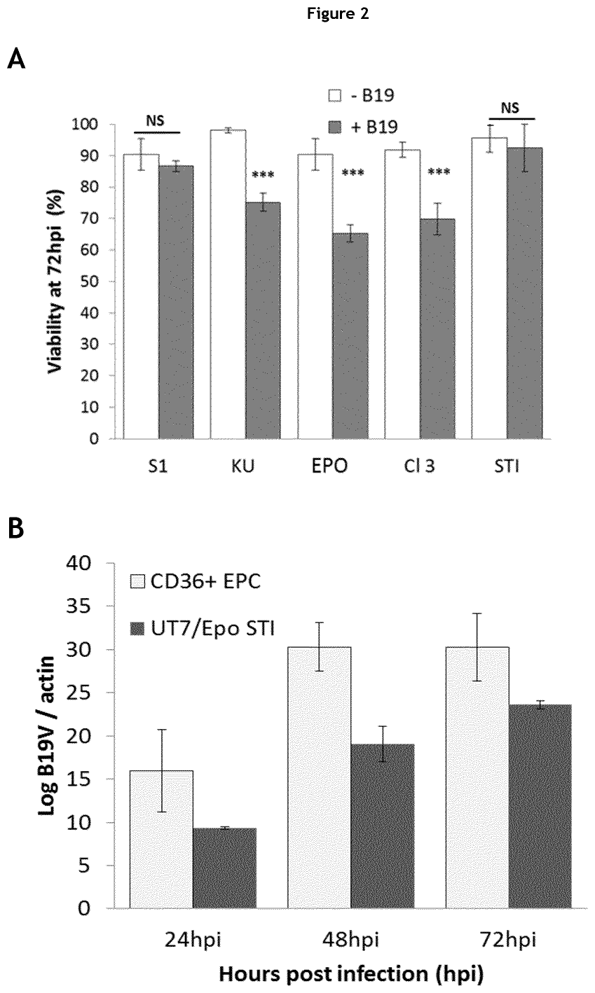

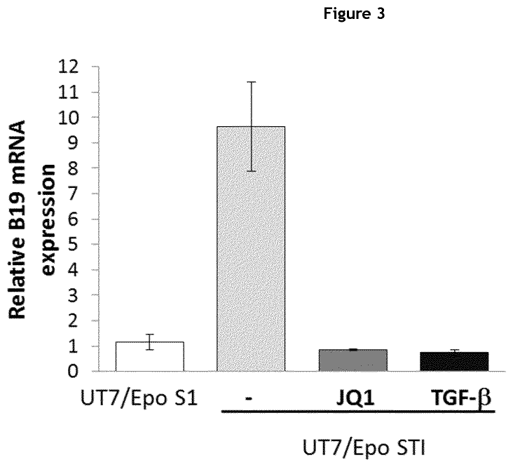

Among all UT7/Epo cell lines tested, UT7/Epo-STI was the UT7/Epo cell line tested with the highest sensitivity to B19V, with B19 mRNA levels 11.8+0.2 times higher than those in UT7/EpoS1. Sensitivity was enhanced by chloroquine treatment and reaches an equivalent level compared to CD36⁺ EPCs (UT7/Epo-STI+CQ: 25.8+4.9 vs. CD36⁺ EPCs 21.49+2.7). This increase in sensitivity was not due to resistance to B19V-induced cytotoxicity (FIG. 2A). The expression kinetics of UT7/Epo-STI B19V were similar to those for CD36⁺ EPC, with a maximum reached at 72 hours post-infection for both cell lines (FIG. 2B). Sensitivity to B19V is directly linked to maturation stage. We therefore subjected UT7/Epo-STI cells to the chemical (JQ1) or hormonal (TGF-B) induction of erythroid differentiation two days before B19V infection. Both treatments decreased B19V infection by a factor of about 10, to levels similar to those obtained for UT7/Epo-S1 (FIG. 3).

The cell cycle is known to be crucial for erythroid differentiation, ensuring precise coordination of the critical differentiation process by Epo and erythroid-specific transcription factors. We hypothesized that sensitivity to B19V may be correlated cell cycle status. We thus chose to select clones according to cell cycle status. UT7/Epo-STI cells were transduced with FUCCI lentiviral particles to generate the UT7/Epo-FUCCI cell line (FIG. 4A). The FUCCI cell cycle sensor allows cell cycle analysis of living cells. The UT7/Epo-FUCCI cell line presents three different colour profiles, from green, corresponding to the S, G2 and M phases, to red, consequent to G1 phase, with a green plus red (yellow) overlay indicating the G1-to-S transition. We checked that these dynamic color changes correctly represented progression through the cell cycle and division, by staining the DNA content of UT7/Epo-FUCCI cells with Hoechst stain (FIG. 4B). An overlay of the DNA staining and FUCCI profiles resulted in a perfect match between the cell cycle status assigned by the FUCCI technique and that assigned on the basis of DNA content: G1 (red) FUCCI cells were detected at a DNA content of 2N, whereas cells at S-G2-M (green) had DNA content peaks of 2N to 4N, consistent with the expected replication of the DNA replication before mitosis. The G1 /5 transition phase (yellow) population was located at the 2N peak, with a slight shift from G1 cells. Overall, these results confirm that FUCCI is an appropriate cell cycle indicator for UT7/Epo cells. We then generated different UT7/Epo-FUCCI clones, each obtained by limiting dilution and culture from a single fluorescent cell. Unlike the UT7/Epo-FUCCI pool, these clones generated from single cells and 100% of the cells were therefore transduced: the colourless cells of the FUCCI profile correspond to the early G1 (eG1) phase (FIG. 4A) and were included in the G1 phase for the purposes of this analysis. We isolated 156 independent clones. FUCCI-negative clones, accounting for one third of the cells isolated, were excluded. We studied the cell cycle status of FUCCI-positive clones. We defined three types of cell cycle profile in a total of 97 clones: 1) 54 clones presented a cell cycle with more than 60% of the cells in G1 phase (55.7% of clones); 2) 29 clones presented a balanced distribution of cells between the G1 and S/G2/M phases (29.9% of clones); 3) 14 clones had a high percentage of cells in the S/G2/M phases (14.4% of clones). In aim to analyse these three types of cell cycle profile, we selected 11 isolated clones in regards to the diversity of their cell cycle patterns (FIG. 5). All these clones had cell cycle patterns that remained stable over time.

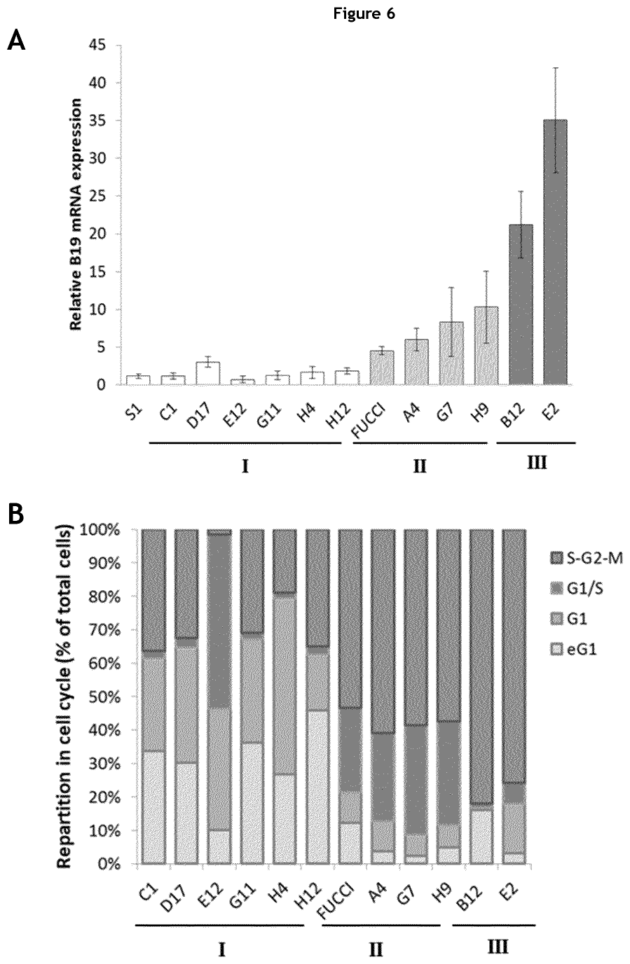

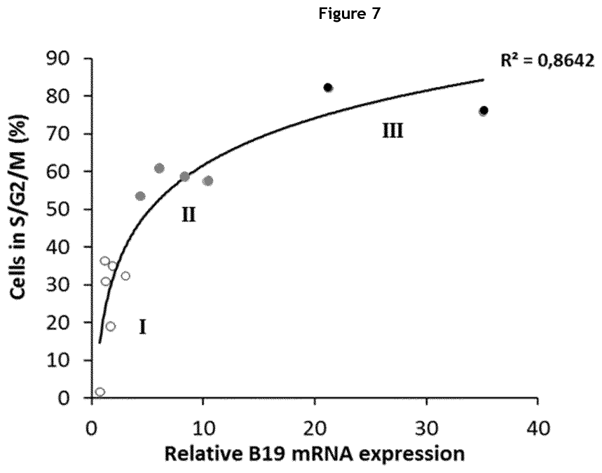

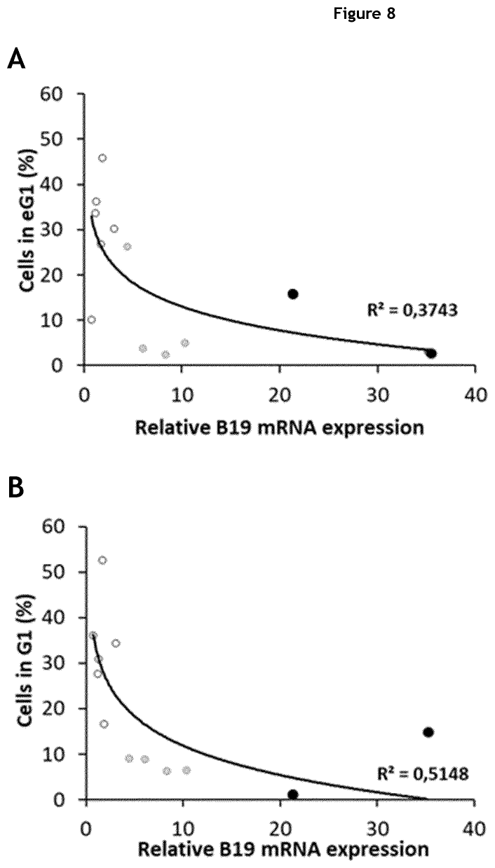

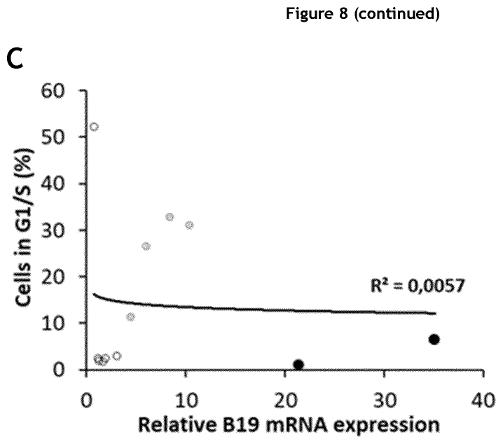

We then evaluated sensitivity to B19V (FIG. 6A). Permissivity ranged from 1-fold to 35-fold relative to UT7/Epo-S1. Three populations were assigned: group I, with a sensitivity close to that of UT7/Epo-S1 (six clones); group II, gathering clones with UT7/Epo-FUCCI-like permissivity (three clones); group III, containing clones B12 and E2, displaying remarkable sensitivity to B19V infection. Interestingly, classification based on B19V sensitivity seemed to group together clones with similar cell cycle patterns (FIG. 6B). The cell cycle profiles of group I clones displayed a predominance of the G1 phase. Group II clones displayed a balance between the G1 and S/G2/M phases, as observed for the original UT7/Epo-FUCCI pool. Finally, the S/G2/M cell population predominantly represents the group III profile, with 82% and 75.8% for clones B12 and E2 respectively. We evaluated the correlation between cell cycle stage and B19V sensitivity, by analysing the correlation of the coefficient of determination (R²) obtained for eG1, G1, G1 /S and S/G2/M with B19V mRNA levels (FIGS. 7 and 8). For G1 cell cycle parameters, R² was low, with values of 0.3743 for early G1 (eG1) and 0.5148 for G1. The highest value was obtained for the S/G2/M phase of the cell cycle, with R²=0.8642, demonstrating an excellent agreement between the percentage of cells in S/G2/M stage and B19V sensitivity (FIG. 6B). Overall, our results identify two highly permissive UT7 clones, B12 and E2, and show that the S/G2/M phase is essential for B19V sensitivity.

### 1.3. Discussion

Most of the currently available approaches focus on the detection of B19V DNA, but there is a need for a suitable in vitro method for the direct quantification of virion infectivity, for use to assess neutralizing antibodies, to evaluate viral inactivation assays or in antiviral research field. However, efforts to develop such methods have been hampered by the lack of suitable B19-sensitive cell lines in vitro. We describe here a new cell model with high sensitivity to B19V infection. As expected, hematopoietic cell lines of different origins were heterogeneous but, surprisingly, our results also demonstrate considerable variability among cell lines derived from the same patient, as all named UT-7/Epo. This variability of B19V sensitivity may depend on erythroid stage, B19V entry receptor expression and/or the activation of specific signaling pathways (7). Our findings highlight the need for tracking criteria to ensure the stability of the cell line used. As we show here that B19V sensitivity is linked to S/G2/M cell cycle status, we propose the use of cell cycle status to define the optimal cells for selection and as a keeper of clone stability. This study proposes an improved cellular model for the detection of B19V infectious units, with a sensitivity 35 times higher than previously achieved. B19V has an extremely strong tropism for human erythroid progenitor cells. Since the discovery that B19V inhibits erythroid colony formation in bone marrow cultures by inducing the premature apoptosis of erythroid progenitor cells, numerous approaches and studies attempt to find a method of virus culture in vitro. Primary or immortalized CD36⁺ erythroid progenitor cells (EPC) derived from hematopoietic stem cells were the most permissive cell models for B19V infection (21). CD36⁺ EPCs reflect the natural etiologic B19V cell host, but the main problem with the use of this model is the difficulty obtaining a continuously homogeneous cell line, with respect to differentiation stage, proliferation rate and metabolic activity. Moreover, the reagents and cytokines required for cell culture (SCF, Il-3, Il-6, Epo) preclude the use of CD36⁺ EPCs for routine B19V cell-based detection methods. To counteract this lack of suitability, cancer cell lines constitute a sound, practical, cost-effective alternative model, overcoming these difficulties. During past years, many cancer cell lines have been tested, but only a few erythroid leukemic (KU812) (15) or megakaryoblastoid cell lines (UT-7)(16) with erythroid characteristics support B19V replication. In our study, we chose also to investigate TF-1 permissivity. The TF-1 cell line is derived from the bone marrow aspirate of an erythroleukemic patient (12). These cells display marked erythroid morphological and cytochemical features common to CD36⁺ EPCs, and the constitutive expression of globin genes highlights the commitment of the cells to the erythroid lineage. Surprisingly, Gallinella et al. showed that TF-1 cells allow only B19V entry, with impaired viral genome replication and transcription, as shown by the presence of single-stranded DNA, and the absence of double-stranded DNA and RNA in B19V-infected TF-1 cells (14). As previously described, no B19V RNA was detectable in the TF-1 cell line. The cellular factors involved in the transcriptional activation of the B19V promoter contribute to the restriction of permissiveness. Two factors, erythropoietin (Epo) and STAT-5, are key factors involved in B19V replication and transcription. TF-1 cells express a truncated and mutated form of the Epo receptor, leading to impaired STAT-5 activation. In the TF-1-ER cell line, stable ectopic expression of a full-length Epo receptor restores Epo-induced proliferation and STAT-5 activation. Here, despite Epo receptor signaling and STAT-5 activation, we found no evidence of B19V transcription, reflecting the involvement of unknown processes in the molecular mechanisms controlling B19V permissivity.

The first cell line reported to be permissive for B19 infection was an Epo-dependent subclone of UT-7, a megakaryoblastoid cell line. In 2006, Wong et al. published a comparative study of B19V sensitivity and permissivity in various cell lines (22). They obtained evidence for the B19V infection of UT7/Epo and KU812Ep6 cells, although the percentage of B19V-positive cells was low (<1% immunofluorescent B19V⁺ cells).

UT7/Epo-S1, a subclone of UT7/Epo obtained by limiting dilution and screening for B19V susceptibility (16), had the highest sensitivity, with approximately 15% of the cells staining positive for B19V (18). Permissivity is restricted to a subset of cells, but the degree of viral DNA replication in these cells is similar to that in EPCs. Since its characterization, the UT7/Epo-S1 cell line has been widely used to investigate the molecular mechanisms of B19V infection and to develop antiviral strategies against B19. We used UT7/Epo-S1 as a reference, and compared the sensitivity of UT7/Epo cells from different laboratories. B19V permissivity seemed to be similar in the various UT7/Epo cells, but UT7/Epo-STI cells displayed levels of B19V gene expression almost 10 times higher than those in UT7/Epo-S1 cells. UT7/Epo-STI cells have been cultured with great care to ensure the preservation of their erythroid features, and they undergo erythroid differentiation following treatment with JQ1, a Bet-domain protein inhibitor or TGF-B1. However attempts to characterize cell lines have been hampered by the heterogeneity of continually evolving multiple subclonal leukemic populations, as revealed at the cytogenetic level by the unstable karyotype documented at various time points for UT-7: at the admission of the patient to hospital (44 chromosomes, XY), at the first cell line characterization (92+/−6 chromosomes, XXYY)(27), in subsequent publication (82+/−4 chromosomes, XXYY and in our own cell line in 2017 (72+/−13 chr., XXYY; unpublished data). This karyotype heterogeneity highlights the presence of heterogeneous subclones within cell lines, and might account for the variation of B19V sensitivity among UT-7 cell lines and clones.

The cell cycle is known to be crucial for erythroid differentiation, ensuring precise coordination of the critical differentiation process by Epo and erythroid-specific transcription factors. We decided to select clones on the basis of cell cycle status. The FUCCI system represents a convenient approach to track cell cycle as its readability allows analyse of living cells at a single cell level. By using clones with different cell cycle status, we demonstrated a strong correlation between S/G2/M cell cycle status and permissivity.

B19V has been shown to induce cell cycle arrest at G2 phase, but the importance of cell cycle status for B19V entry has not been investigated. A complex combination of multiple factors, including differentiation stage, specific cell cycle status, surface receptor and co-receptor, signaling pathways and transcription factors, may account for the difficulty identifying the best cellular model for completion of the B19 viral cycle. We describe here two clones, E2 and B12, with a permissivity for B19 35 times higher than that of the previously described references. By comparison with their less sensitive counterparts (groups I Et II), these new highly permissive cell models (group III) constitute a potential advance towards understanding the crucial molecular determinants of B19V infectivity.

In addition to the use of E2 and B12 clones to investigate the molecular mechanisms of B19 infection, cell-based methods can be used for the detection/quantification of B19 infectious units, at low levels (<10⁴ DNA geq), in human fluids and tissues. There is a need for a practical in vitro method for the direct quantification of virion infectivity, as applied for the screening and/or assessment of neutralizing antibodies, antiviral drugs and viral inactivation assays.

In the context of plasma-derived medicinal products, due to the lack of a suitable in vitro culture assay for B19, animal parvoviruses are currently used as a model for B19V, to assess B19 viral reduction during manufacturing processes. However, it remains unclear whether these models accurately reflect the behaviour of B19V. Animal model parvoviruses display a certain resistance to heat inactivation and pH stability, but comparative studies have indicated that they may behave differently from human B19. As E2 and B12 were the most sensitive cells in our study, with a permissivity 35 times higher than that of previously established references, they could allow the use of human parvovirus for the testing of viral inactivation processes, and the results of these tests would reflect the behaviour of the native human virus.

Given the severity of B19V infection in immunocompromised patients, the development of antiviral strategies and drugs directed against B19V should require the highest relevance. Depending on the immune state of the infected patient, acute infections can be clinically severe, and an impaired immune response can lead to persistent infections. The administration of high-dose intravenous immunoglobulins (IVIG) is currently considered the only available option for neutralizing the infectious virus. In addition to the use of IVIG, the discovery of antiviral drugs with significant activity against B19 would offer important opportunities in the treatment and management of severe clinical manifestations. Two factors have critically limited the search for compounds to date. Firstly, the lack of a standardized and sensitive in vitro cell culture model has hampered advances in this field. Due to its usefulness, practicability and sensitivity, our cell model could replace the use of CD36⁺ EPCs and UT7/Epo-S1 cells in the discovery and evaluation of antiviral candidate compounds. Secondly, antiviral research requires native B19 infectious particles. But B19V particles from viremic patients limit the feasibility of high-throughput screening against the available chemical libraries. No appropriate system for cell culture and in vitro virus production were available to date. UT-7 cells have been reported to produce infectious viral particles in vitro (but only a few UT-7 cells are infected and virions are produced in small numbers. The strategies used here for the selection of clones permissive for B19V could also be used to select even more productive clones, in particular by single cell cloning of UT7/Epo-FUCCI and selection of clones with the highest proportion of cells in the one of the S/G2/M phases. Altogether, we propose here an improved cell model with a high degree of permissivity to B19V, allowing the sensitive detection of infectious particles of B19. This finding opens up challenging new perspectives for basic research on B19V life cycle. It may also offer opportunities for improving key steps in a number of critical applied approaches, including the sensitive evaluation of B19V virions in manufactured blood-derived products, and new strategies for B19V production in vitro.

### 2. Example 2: Characterization of UT-7/Epo-STI Cell Line and of the Selected Clones

### 2.1. Materials and Methods

### 2.1.1. Cell Lines

UT7/Epo-S1 and UT7/Epo-STI cells are as in example 1 (see sections 1.1.1 and 1.2 above). UT7/Epo-FUCCI cells are as in example 1 (see sections 1.1.5 and 1.2 above). UT7/Epo-E2 is E2 clone as in example 1 (see section 1.2 above).

### 2.1.2. Evaluation of Response to Cytokines

### 2.1.2.1. RT-qPCR

Expression of Receptor for Erythropoietin (Epo-R) and GM-CSF (GM-CSF-R) mRNA was evaluated by RT-qPCR. Briefly, for each cell lines, exponentially growing cells were collected by centrifugation. RNA was extracted from the cell pellet, and mRNA levels were analysed by RT-qPCR to quantify Epo-R and GM-CSF-R expression. 18S rRNA levels were analysed for cell number normalization. Taqman primers (Thermofisher Scientific) used are Epo-R (Hs00959427-m1), GM-CSF-R-alpha (Hs00531296-g1) and 18S (Hs99999901-s1). Relative threshold cycle (Ct) values were normalized to the 18S Ct (log mRNA/18S). The results shown are the means+/−SEM of three independent experiments performed with triplicates.

### 2.1.2.2. Western Blot of Total Cell Extracts

Starved Cells (UT7/Epo-S1, UT7/Epo-STI, UT7/Epo-FUCCI) were stimulated with Epo (E: 10U/mL), GM-CSF (GM: 25ng/mL) or TPO (100 ng/mL) or left unstimulated (−). After lysis, cell extracts were analysed by western-blot using antibodies raised against total (α-STAT-5, Cell Signaling Technology cat. No 94205) or phosphorylated forms of STAT-5 (α-pSTAT-5, Cell Signaling Technology cat. No 9351), and B23 for cell extract normalization (α-B23, Santa Cruz Biotechnology cat. No 271737).

### 2.1.3. Proliferation Capacities

At day 0, exponentially growing cells were cultivated in culture media at a starting concentration of 1.10⁵ cells/mL and incubated until 7 days at 37° C., 5% CO₂. Each day, cell concentration was calculated by counting cells under microscope using an hemacytometer. To exclude dying cells, Trypan blue exclusion dye is added to cell suspension before counting, and only non-colored cells were counted.

### 2.1.4. Permissivity to B19 Infection

### 2.1.4.1. Evaluation of B19 Genome Transcription (B19 ARN) and Replication (B19 DNA)

Total nucleic acids were extracted from cell pellets with the RNeasy 96 QIAcubeHT kit and a QIAcubeHT machine, according to the manufacturer's instructions. The final extraction step included:
a) for B19 genome replication, a RNAse treatment to remove RNA for DNA analysis;
b) for B19 transcription evaluation, a DNase treatment for 15 min, to remove DNA and keep RNA.

A reverse transcription step ensure the production of cDNA.

Quantitative PCR (qPCR) is then performed with the Taqman Fast Virus one-step PCR kit (Applied Biosystems). B19 VP2 transcripts were amplified with the sense primer B19-21 5′-TGGCAGACCAGTTTCGTGAA-3′ (nts 2342-2361; SEQ ID NO: 1), the antisense primer B19-22 5′-CCGGCAAACTTCCTTGAAAA-3′ (nts 3247-3266; SEQ ID NO: 2) and the probe B19-V23 5′-VIC-CAGCTGCCCCTGTGGCCC-3′ (nts 3228-3245; SEQ ID NO: 3). For control and normalization with respect to the number of cells, we used a duplex strategy. A target sequence of the spliced beta actin transcript was selected and amplified with the sense primer actin-S 5′-GGCACCCAGCACAATGAAG-3′ (SEQ ID NO: 4), the antisense primer actin-AS 5′GCCGATCCACACGGAGTACT-3′ (SEQ ID NO: 5) and the probe actin-FAM 5′-FAM-TCAAGATCATTGCTCCTCCTGAGCGC-3′ (SEQ ID NO: 6). Reactions were performed on 5 μL of extracted nucleic acids with the Quant Studio 3 PCR system. The reaction began with activation of the polymerase by heating at 95° C. for 10 min, followed by 40 cycles of 15 s at 95° C. and 30 s at 60° C. The PCR program was optimized for amplification of the VP2 spliced transcripts rather than the VP2 genomic sequence (FIG. 1A).

### 2.1.4.2. Production of B19 Genome Copies Equivalent (Geq) per mL of Cell Culture

Cells (day 8 CD36⁺EPC, UT7/Epo-S1, UT7/Epo-STI and UT7/Epo-E2) were inoculated with B19. 24 h after inoculation (24 hpi), cells were washed. 3 days later (96 hpi), supernatant was collected, DNA was isolated and B19 DNA was quantified by qPCR according to a B19 DNA standard (GenBank accession no. AY386330). Results are means±SD of 3 independent experiments. For CD36+ EPC, results are means±SD of three day-8 erythroid culture from CD34+ hematopoietic stem cells isolated from 3 different umbilical cord blood.

### 2.1.5. RNA Seq

Total RNA extraction was extracted from 3 independent cultures and with TRIzol reagent and the Purelink RNA kit (Ambion). The quality of the RNA was checked with an Agilent 2100 Bioanalyzer before analysis. Libraries were prepared at Active Motif Inc. using the Illumina TruSeq Stranded mRNA Sample Preparation kit, and sequencing was performed on the Illumina NextSeq 500 as 42-nt long-paired end reads (PE42). Fastp (v. 0.19.5) was used to filter low quality reads (Q >30) and trim remaining PCR primers. Read mapping against the human genome (hg19) was done using HISAT2 (v. 2.1.0) and fragment quantification was done using string tie (v. 2.1.1). Differential gene expression analysis was performed using the DESeq2 R package. The Wald test was performed for pair-wise comparison, and genes were considered significantly differentially expressed if absolute value of their log2 fold change (FC) was over 1 and if FDR (False Discovery Rate) was less than 0.05.

### 2.1.6. Analysis of B19V Receptor in UT-7 Cell Lines, Integrin-α5 (CD49e)

Analysis of CD49e expression on UT7 cells was performed by flow cytometry after labelling with anti-CD49e antibody crosslinked to APC (allophycocyanin) fluorescent marker (Invitrogen MA5-23585) at different concentrations. After washing, fluorescence was subsequently analysed with a LSRFortessa cytometer (BD Biosciences) with the 633 nm red laser and a 670/14 nm bandpass filter. Unstained cells and cells stained with an isotype antibody (IgG-APC) are used as negative controls.

### 2.2. Results

### 2.2.1. Response to Cytokines

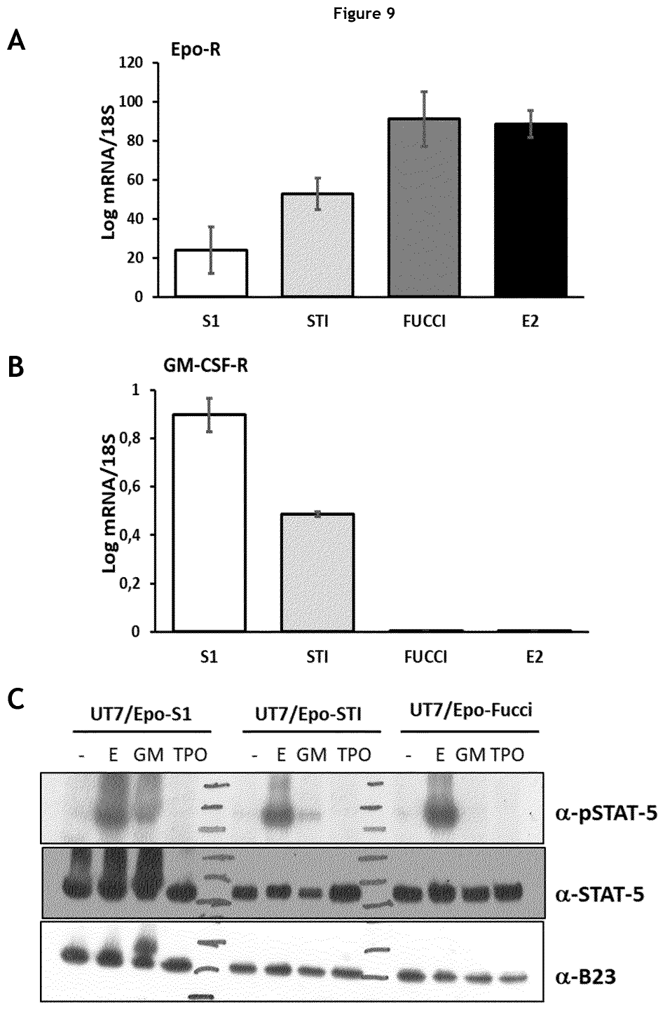

Expression of Receptor for Erythropoietin (Epo-R; FIG. 9A) and GM-CSF (GM-CSF-R; FIG. 9B) mRNA was evaluated by RT-qPCR. In addition, starved Cells (UT7/Epo-S1, UT7/Epo-STI, UT7/Epo-FUCCI) were stimulated with Epo (E: 10 U/mL), GM-CSF (GM: 25 ng/mL) or TPO (100 ng/mL) or left unstimulated (−) were analyzed by western-blot (FIG. 9C). Results obtained demonstrate that Epo-R signaling is conserved in all three cell lines, whereas GM-CSF-R induced STAT-5 phosphorylation is activated in UT7/Epo-S1, significantly reduced in UT7/Epo-STI and undetected in UT7/Epo-Fucci cells (FIG. 9). These results are consistent with the expression of relative receptors as presented in FIG. 9B.

### 2.2.2. Proliferation Capacities of UT-7/Epo-STI Cell Line and of the Selected Clone E2

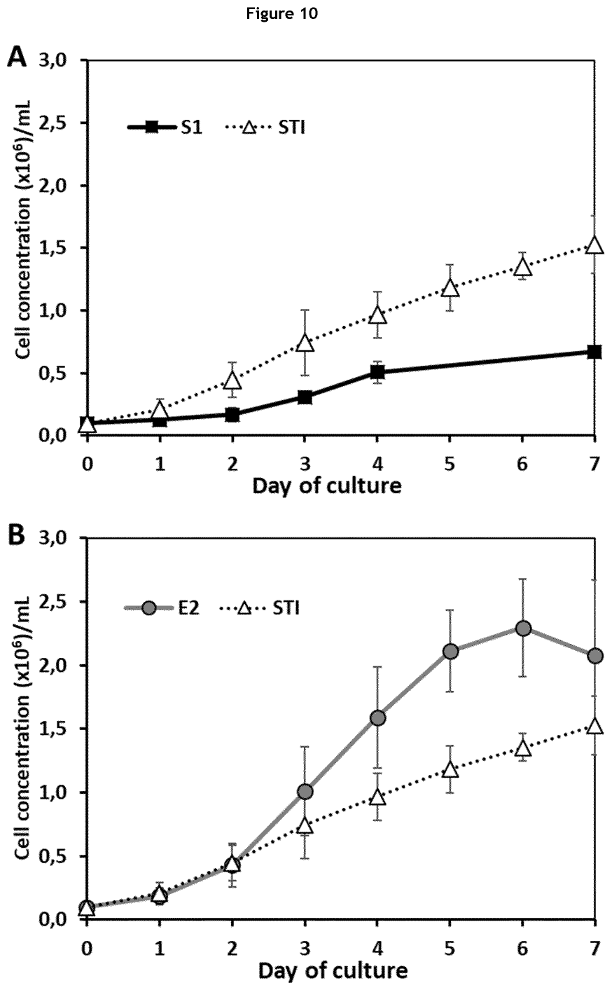

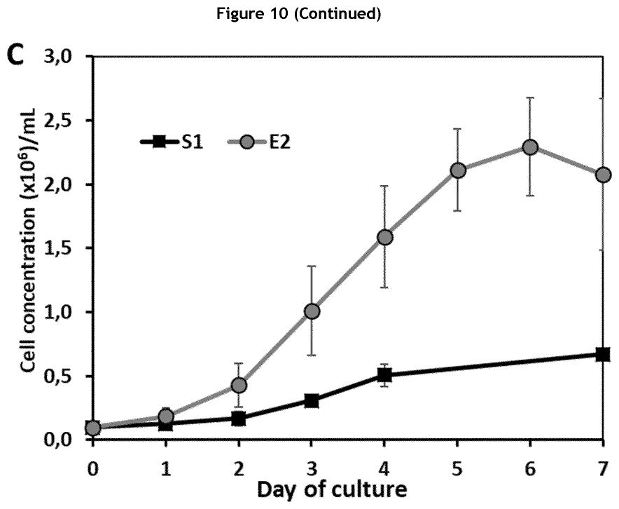

Cell proliferation capacity was evaluated by cell counting of live cells. The data show that proliferation is significantly higher in UT-7/Epo-STI cell line, compared to UT-7/Epo-S1 cell line. Proliferation is significantly enhanced in UT7/Epo-STI-derived clone E2 (FIG. 10). These results suggest that UT-7/Epo-S1 cells are less sensitive to Epo for cell growth than UT-7/Epo-STI and UT-7/Epo-E2. Those data are in correlation with UT-7 cells response to cytokines (FIG. 9).

### 2.2.3. Permissivity of UT-7/Epo-STI Cell Line and of the Selected Clones

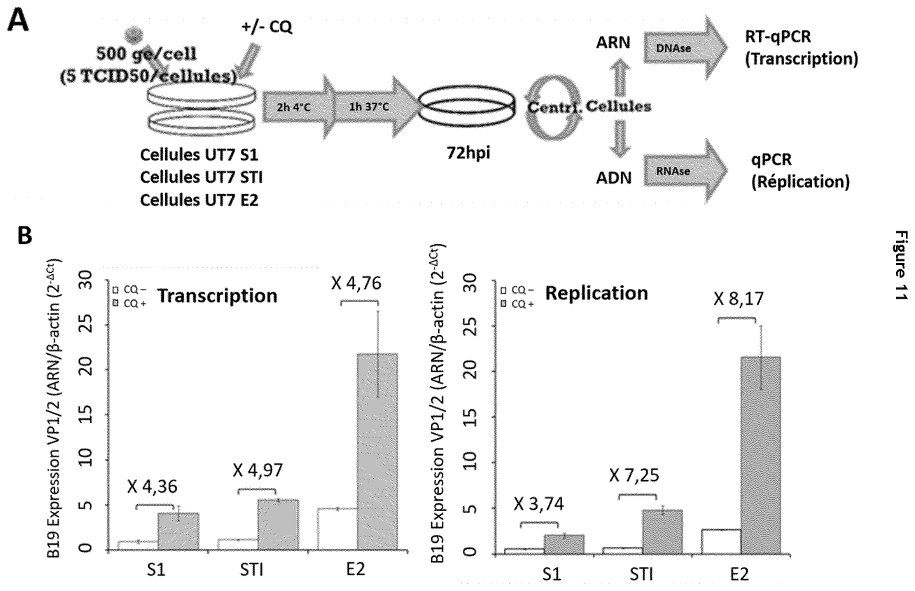

B19 genome transcription and replication were evaluated by RT-qPCR at 72 h post-infection. The data show that B19 genome transcription (mRNA; FIG. 11B) and replication (DNA; FIG. 11C) are significantly higher in UT-7/Epo-STI cell line, compared to UT-7/Epo-S1 cell line, confirming that UT-7/Epo-STI cell line is more permissive to B19 infection compared to UT-7/Epo-S1. UT7/Epo-E2 clone possesses the highest B19 genome transcription and replication levels, corroborating the excellent permissivity to B19 infection of this new cell line.

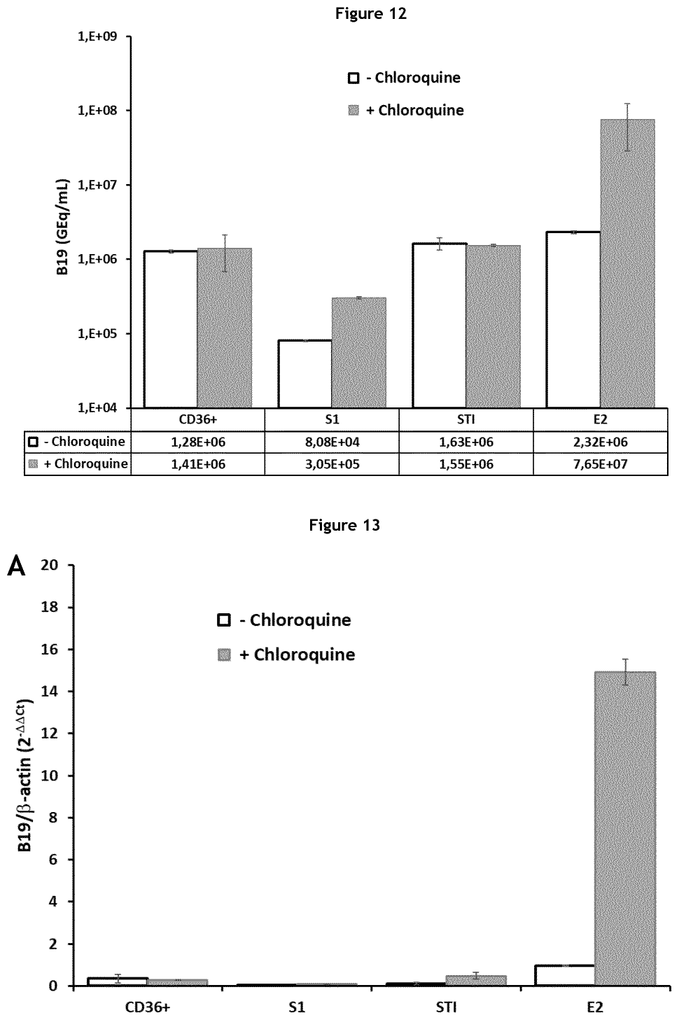

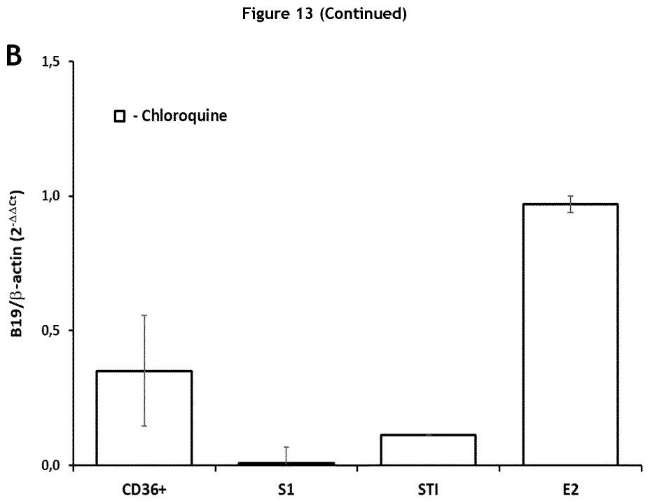

B19 genome production (FIG. 12) and transcription (FIG. 13) was evaluated at 96h post infection in CD36+EPC cells (day 8) and UT7 cell lines with or without addition of chloroquine (FIG. 13A and B). 96 h post infection, DNA was extracted from an aliquot of 1 mL of cell culture and RNA from cell pellets. B19 genome production was evaluated by qPCR and genome equivalent (GEq)/mL was calculated according to a B19 genome calibration curve. Transcription was analysed by RT-qPCR as previously described. FIG. 12 demonstrate that UT-7/Epo-S1 produced the lower yield of B19 GEq/mL (around 4 to 5) while CD36+ EPC and UT-7/Epo-STI show a yield reaching 6 log. UT7/Epo-E2 cell line presents the highest quantity of B19 genome equivalent produced, with at least 7.65×10⁷ GEq/mL after chloroquine treatment. For the same culture, transcription of B19 genome (FIG. 13A and 13B for magnification of Y-axis without chloroquine) seems to reach the highest level for UT7/Epo-E2 treated with chloroquine, thus corroborating the excellent permissivity to B19 infection of this new cell line.

### 2.2.4. RNA Sequencing of UT-7/Epo-STI Cell Line and of the Selected Clones

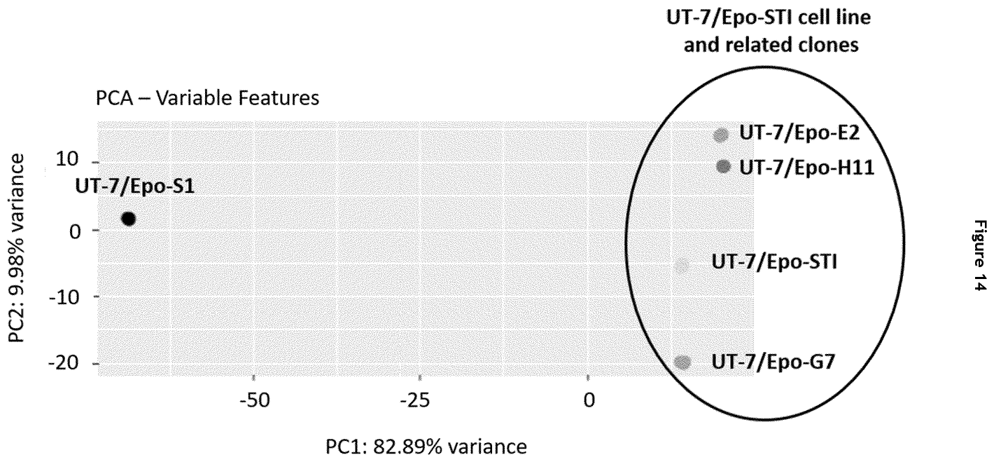

RNA sequencing of transcriptomes was used to evaluate the molecular signature of each cell lines. UT7/Epo-S1, UT7/Epo-STI cell line and 3 different UT-7/Epo-STI derived clones (E2, H11 and G7) whole transcript were analyzed by RNA sequencing. FIG. 14 shows the repartition of all the data in a two-dimension scale as PCA (Principle Component Analysis). PCA is a mathematical transformation to reduce the dimensionality of data. The high dimensional expression data is converted to a set of new variables called Principle Components. Principle component 1 (PC1) accounts for the most amount of variation cross samples, PC2 the second most, and so on. The PCA plot summarizes the expression values for each cell lines (in triplicate) in the 2D plane of PC1 and PC2. As shown in FIG. 14, UT-7/Epo-S1 segregates in the PC1 plane from the other UT-7 cell lines, with 82.89% of variance, clearly demonstrating that UT-7/Epo-STI cell line and derived clones are strictly distinguishable from UT-7/Epo-S1 cell line. In the opposite, UT7/Epo-STI derived clones segregate in the same PC1 plane, with a PC2 plane of 9.98% variance, demonstrating common shared characteristics between UT-7/Epo-STI derived cell lines and clones.

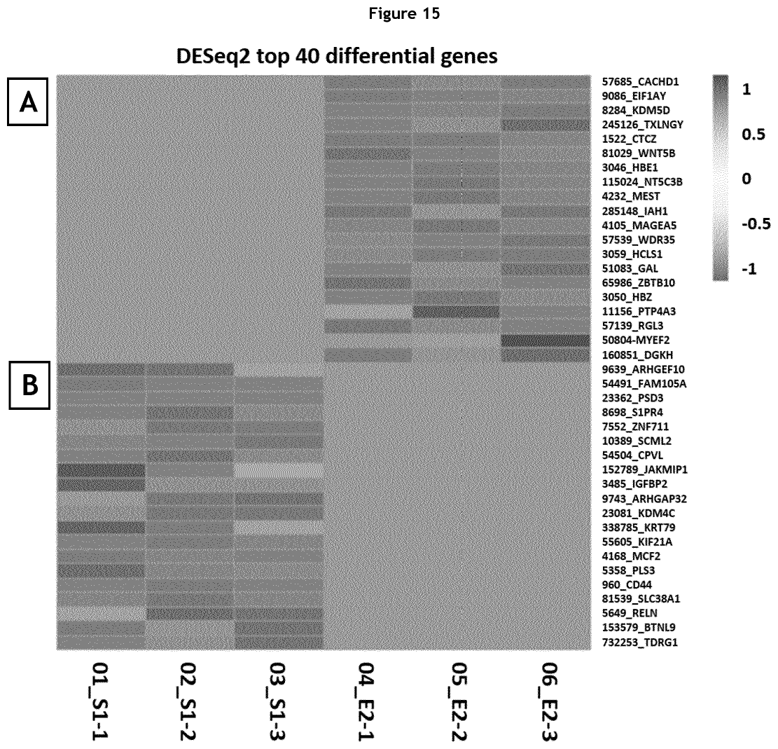

FIG. 15 corresponds to the heatmap of top 40 differential genes expressed between UT7/Epo-S1 and UT7/Epo-E2. After row standardization (i.e. data scaling with a mean of zero and a standard deviation of one), each row is a gene and each column is a sample. Entrez gene identifier and symbol are also shown in the heatmap for top 40 differential genes, equally distributed between up (FIG. 15A) and down (FIG. 15B) regulated genes in UT-7/Epo-E2 versus S1 cell line. This figure demonstrate that molecular signature and subset of genes permits to distinguish UT-7/Epo-E2 from UT-7/Epo-S1.

Table 1 and 2 show subsets of genes respectively up and down regulated in UT7/Epo-E2 cell lines compared to UT-7/Epo-S1, providing a list of candidate gene illustrating the erythroid engagement of UT7/epo-E2 cell line, where erythroid-related pathways are up-regulated (table 1) and non-erythroid related pathways are down-regulated (table 2). Altogether, those date demonstrate that UT-7/Epo-S1 are distinguishable from UT-7/Epo-STI cell lines and derived clones, and corroborates the erythroid engagement of UT-7/Epo-E2.

TABLE 1
RNA sequencing of UT7/Epo-E2 compared to UT7/Epo-S1: List of up-regulated genes in UT7/Epo-E2
versus UT7/Epo-S1 (among 5747 up-regulated genes) related to erythroid specification.
Data (Fold change, FC) are expressed as means ± SD of 3 independent experiments.

| Gene | Gene | | | | | | | Log2 FC | pValue | |
|---|---|---|---|---|---|---|---|---|---|---|
| ID | Symbol | S1-1 | S1-2 | S1-3 | E2-1 | E2-2 | E2-3 | E2 vs S1 | E2 vs S1 | Pathway |
| 48 | ACO1 | 1272 | 1356 | 1304 | 3060 | 3057 | 3107 | 1.25 | 3.67E−166 | Iron response |
| | | | | | | | | | | element |
| 208 | AKT2 | 2935 | 2801 | 2869 | 6849 | 6910 | 6596 | 1.26 | 1.61E−282 | Signaling |
| | | | | | | | | | | pathway |
| 118788 | PIK3AP1 | 1232 | 1148 | 1113 | 2994 | 2939 | 3016 | 1.37 | 1.96E−178 | Signaling |
| | | | | | | | | | | pathway |
| 1536 | CYBB | 118 | 98 | 91 | 249 | 285 | 314 | 1.46 | 1.63E−22 | Glycophorin B |
| 2994 | GYPB | 98 | 85 | 124 | 266 | 307 | 274 | 1.46 | 3.11E−22 | Glycophorin B |
| 5465 | PPARA | 1030 | 950 | 989 | 2741 | 2849 | 2652 | 1.49 | 3.30E−184 | Signaling |
| | | | | | | | | | | pathway |
| 3040 | HBA2 | 2323 | 2424 | 2653 | 7310 | 7637 | 7224 | 1.6 | 7.27E−04 | Hemoglobin |
| | | | | | | | | | | alpha |
| 2993 | GYPA | 1394 | 1458 | 1493 | 4750 | 4446 | 4444 | 1.67 | 3.71E−305 | Glycophorin B |
| 55363 | HEMGN | 3017 | 2885 | 2776 | 10814 | 10923 | 10798 | 1.92 | 4.79E−05 | Hemogen |
| 3077 | HFE | 8 | 9 | 11 | 39 | 53 | 48 | 2.07 | 1.15E−08 | Iron response |
| | | | | | | | | | | element |
| 3044 | HBBP1 | 385 | 393 | 392 | 5256 | 5156 | 5108 | 3.74 | 4.60E−05 | Hemoglobin |
| 3050 | HBZ | 32 | 32 | 40 | 4404 | 4509 | 4287 | 6.9 | 1.16E−04 | Hemoglobin |
| 3046 | HBE1 | 620 | 644 | 648 | 204700 | 207309 | 203586 | 8.34 | 1.45E−05 | Hemoglobin |

TABLE 2
RNA sequencing of UT7/Epo-E2 compared to UT7/Epo-S1: List of down-regulated genes in UT7/Epo-E2
versus UT7/Epo-S1 (among 6749 down-regulated genes) related to hematopoietic signaling.
Data (Fold change, FC) are expressed as means ± SD of 3 independent experiments.

| Gene | Gene | | | | | | | Log2 FC | pValue | |
|---|---|---|---|---|---|---|---|---|---|---|
| ID | Symbol | S1-1 | S1-2 | S1-3 | E2-1 | E2-2 | E2-3 | E2 vs S1 | E2 vs S1 | Pathway |
| 960 | CD44 | 2915 | 2949 | 2906 | 2 | 2 | 0 | −9.25 | 1.69E−151 | Immature |
| | | | | | | | | | | hematopoiesis |
| 5358 | PLS3 | 2490 | 2349 | 2309 | 0 | 2 | 0 | −9.18 | 1.21E−131 | Immature |
| | | | | | | | | | | hematopoiesis |
| 3485 | IGFBP2 | 1715 | 1538 | 1523 | 3 | 0 | 0 | −8.55 | 3.29E−116 | IGF signaling |
| 2260 | FGFR1 | 701 | 702 | 676 | 1 | 5 | 2 | −6.99 | 1.73E−92 | Fibroblast |
| | | | | | | | | | | receptor |
| 970 | CD70 | 1903 | 1959 | 1931 | 13 | 15 | 26 | −6.53 | 5.74E−261 | TNF signaling |
| 2621 | GAS6 | 252 | 257 | 279 | 0 | 1 | 0 | −6.5 | 3.92E−49 | Matrix receptor |
| 2833 | CXCR3 | 229 | 207 | 226 | 0 | 0 | 0 | −6.44 | 3.10E−44 | Interleukin |
| | | | | | | | | | | signaling |
| 3553 | IL1B | 1183 | 1157 | 1204 | 9 | 13 | 14 | −6.34 | 6.39E−175 | Interleukin |
| | | | | | | | | | | signaling |
| 53335 | BCL11A | 889 | 858 | 861 | 19 | 9 | 5 | −5.97 | 1.50E−144 | Immature |
| | | | | | | | | | | hematopoiesis |
| 2258 | FGF13 | 1411 | 1381 | 1365 | 20 | 25 | 24 | −5.76 | 2.22E−235 | Fibroblast |
| | | | | | | | | | | Growth Factor |
| 3561 | IL2RG | 6506 | 6343 | 6588 | 181 | 200 | 224 | −4.98 | 5.884E−05 | Interleukin |
| | | | | | | | | | | signaling |
| 3604 | TNFRSF9 | 90 | 98 | 114 | 2 | 1 | 3 | −4.57 | 6.12E−28 | TNF signaling |
| 3560 | IL2RB | 582 | 560 | 579 | 23 | 16 | 29 | −4.52 | 1.03E−124 | Interleukin |
| | | | | | | | | | | signaling |
| 3580 | CXCR2P1 | 97 | 94 | 97 | 1 | 2 | 3 | −4.52 | 3.37E−27 | Interleukin |
| | | | | | | | | | | signaling |
| 3667 | IRS1 | 44 | 61 | 40 | 0 | 0 | 0 | −4.51 | 9.55E−18 | IGF signaling |
| 10225 | CD96 | 1443 | 1442 | 1479 | 102 | 101 | 108 | −3.76 | 2.739E−05 | T-Cell receptor |
| 3488 | IGFBP5 | 247 | 232 | 235 | 17 | 23 | 13 | −3.6 | 2.11E−54 | IGF signaling |
| 3577 | CXCR1 | 107 | 127 | 128 | 10 | 7 | 6 | −3.59 | 7.38E−30 | Interleukin |
| | | | | | | | | | | signaling |
| 10642 | IGF2BP1 | 56 | 50 | 61 | 4 | 2 | 2 | −3.56 | 1.26E−16 | IGF signaling |
| 147920 | IGFL2 | 1545 | 1574 | 1577 | 158 | 161 | 153 | −3.28 | 3.034E−05 | IGF signaling |
| 23765 | IL17RA | 27 | 15 | 16 | 0 | 0 | 1 | −3.11 | 3.59E−08 | Interleukin |
| | | | | | | | | | | signaling |
| 8832 | CD84 | 180 | 150 | 168 | 18 | 19 | 15 | −3.08 | 8.51E−36 | Lymphocyte |
| | | | | | | | | | | signaling |
| 3574 | IL7 | 546 | 571 | 490 | 72 | 65 | 68 | −2.91 | 1.64E−98 | Interleukin |
| | | | | | | | | | | signaling |
| 3557 | IL1RN | 118 | 124 | 113 | 17 | 16 | 12 | −2.79 | 8.09E−25 | Interleukin |
| | | | | | | | | | | signaling |
| 1524 | CX3CR1 | 13 | 13 | 11 | 0 | 0 | 0 | −2.78 | 2.70E−06 | Interleukin |
| | | | | | | | | | | signaling |
| 3815 | KIT | 8489 | 8698 | 8419 | 1521 | 1491 | 1578 | −2.46 | 1.17E−04 | Immature |
| | | | | | | | | | | hematopoiesis |
| 9308 | CD83 | 135 | 144 | 154 | 27 | 27 | 20 | −2.43 | 1.40E−25 | B-Cell signaling |
| 968 | CD68 | 5323 | 5163 | 5233 | 1496 | 1424 | 1384 | −1.85 | 0 | Monocyte/ |
| | | | | | | | | | | macrophage |
| 1439 | CSF2RB | 7006 | 7040 | 6962 | 4508 | 4610 | 4469 | −0.61 | 1.98E−88 | GM-CSF R beta |
| | | | | | | | | | | sous unité |

### 2.2.5. Analysis of B19V Receptor in UT-7 Cell Lines, Integrin-α5 (CD49e)

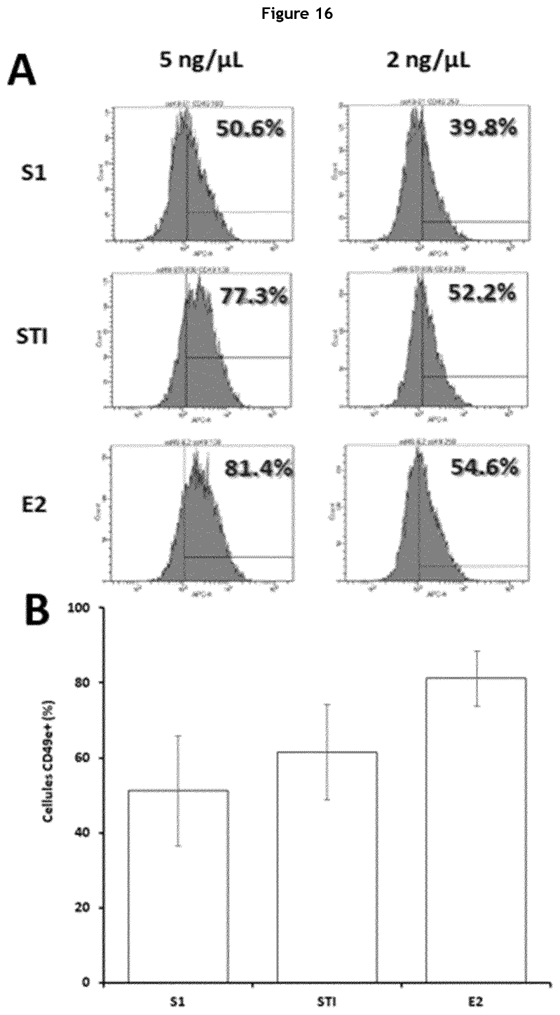

CD49e (Integrin α-5) is a receptor for B19 virus at the surface of host cells, essential for viral particles entry. We have analyzed and compared the expression of CD49e at cell surface of UT-7/Epo-S1, UT-7/Epo-STI and UT-7/Epo-E2 cell lines by cytometry (FIG. 16). Specific fluorescent antibody raised against CD49e was used at two different concentrations (2 or 5 ng/mL). Percentage of CD49e+ cells was measured (FIG. 16A) Results demonstrate that UT-7/Epo-E2 shows the highest percentage of CD49e+ cells, with at least 81% of CD49e+ cells.

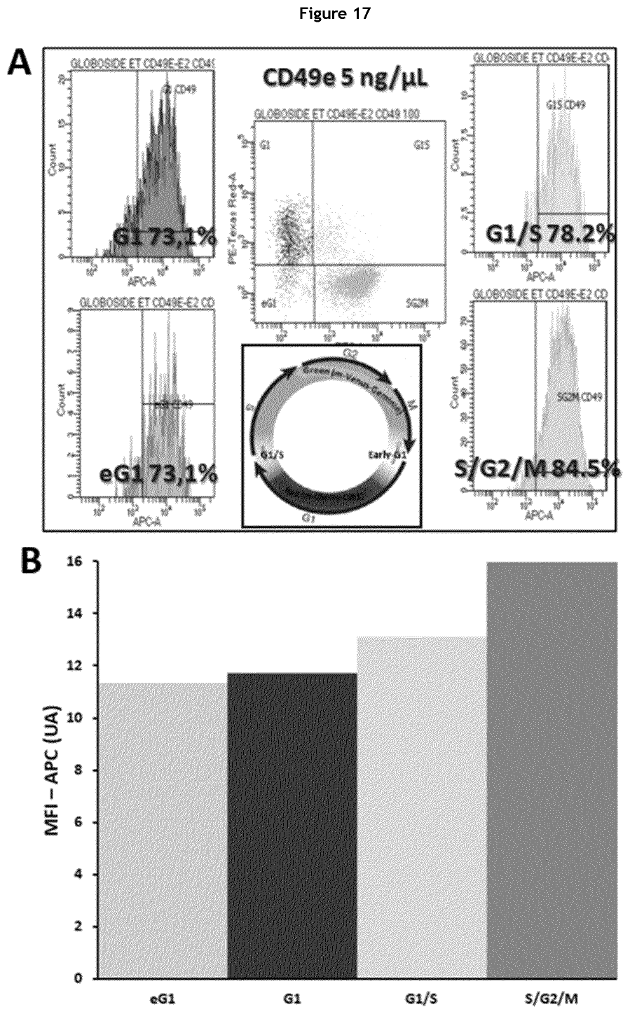

The repartition of these CD49e+ according to cell cycle was analyzed by scoring the CD49e+ cells inside cell cycle phases according to FUCCI repartition (FIG. 17A). To analyze the level of expression of CD49e, mean of fluorescence Intensity (MFI) of CD49e-associated fluorescence was evaluated. Results shows that intensity of CD49e signal increase according to cell cycle phases, with the highest level reached by cells in S/G2/M phase (FIG. 17B). This data suggest that cells in S/G2/M cell cycle phase express more CD49e.

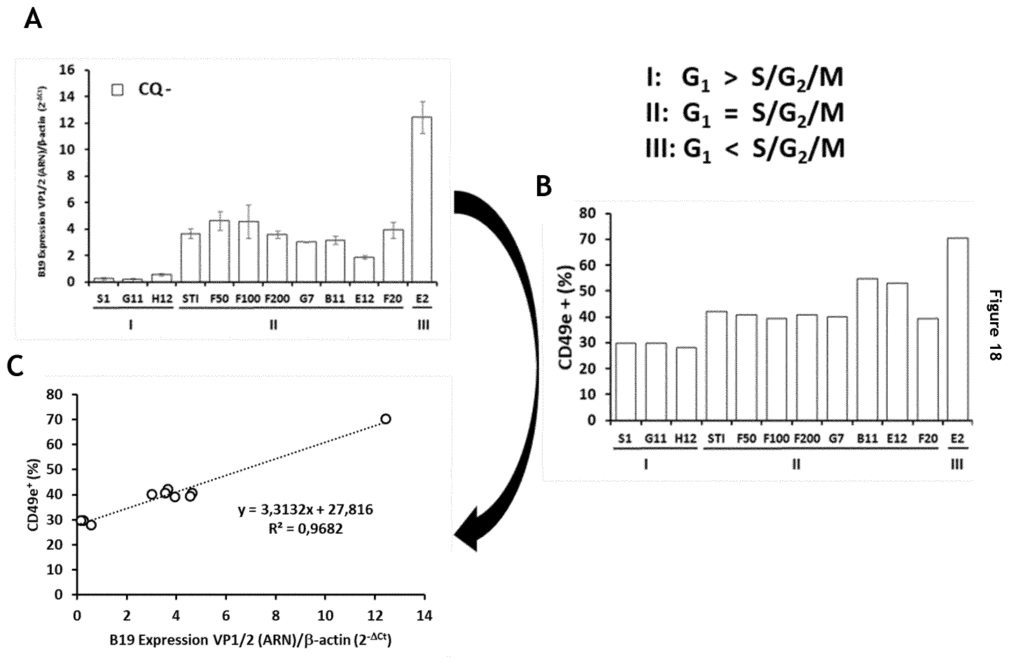

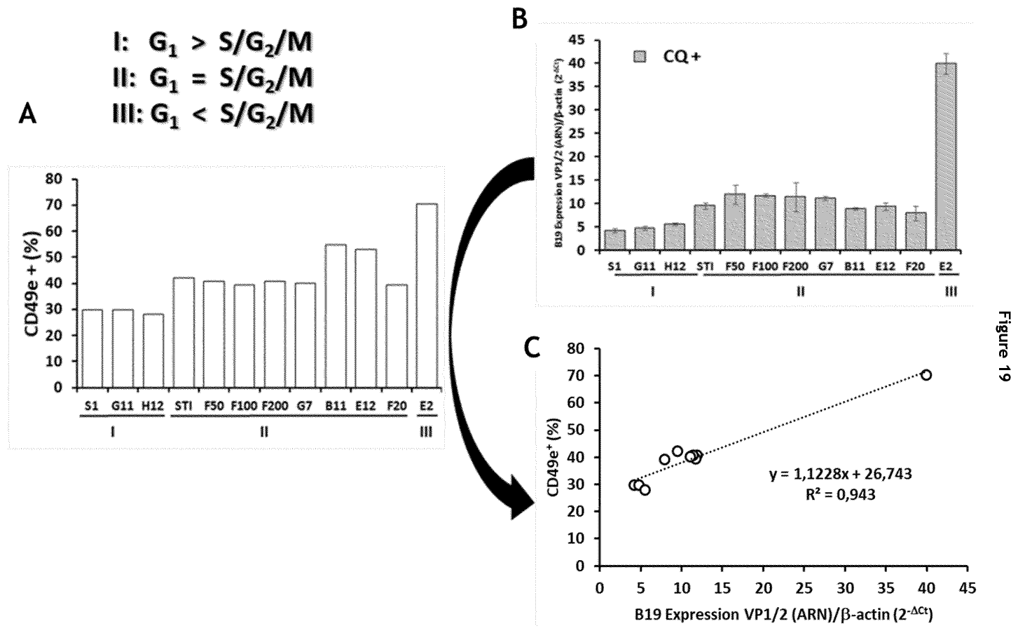

In order to analyze the link between CD49e expression and B19 permissivity, these two parameters were measured in UT-7/Epo-S1, UT-7/Epo-STI and UT-7/Epo-STI derived clones with (FIG. 19) or without (FIG. 18) chloroquine treatment. Clones and cell lines were classified in three groups according to the repartition of their cell cycle. As previously described in FIG. 6, cells showed a graded B19 permissivity as S/G2/M percentage increase, with the highest level reached for E2 cell line (FIG. 18A and 19A). CD49e expression seems to be also correlated with cell cycle (FIG. 18B and 19B), with the highest percentage of CD49e+ cells for E2 cell line. We evaluated the correlation between CD49e expression and B19V sensitivity, by analyzing the correlation of the coefficient of determination (R2) obtained for CD49e+ cells with B19V mRNA levels (FIG. 18C and 19C). Results showed that with or without chloroquine, CD49e expression correlates with B19 permissivity, with the highest value for UT-7/Epo-E2 cell line.

Altogether, these data provide a set of molecular clues (S/G2/M cell cycle phase, CD49e expression, STAT-5 activation and pronounced erythroid features) to the improved permissivity of UT-7/Epo-E2 towards B19 infection.

### BIBLIOGRAPHIC REFERENCES

1. Cotmore S F, Agbandje-McKenna M, Canuti M, Chiorini J A, Eis-Hubinger A M, Hughes J, Mietzsch M, Modha S, Ogliastro M, Penzes JJ, Pintel D J, Qiu J, Soderlund-Venermo M, Tattersall P, Tijssen P, Ictv Report C. 2019. ICTV Virus Taxonomy Profile: Parvoviridae. J Gen Virol 100:367-368.
2. Ganaie S S, Qiu J. 2018. Recent Advances in Replication and Infection of Human Parvovirus B19. Front Cell Infect Microbiol 8:166.
3. Kurtzman G J, Ozawa K, Cohen B, Hanson G, Oseas R, Young N S. 1987. Chronic bone marrow failure due to persistent B19 parvovirus infection. N Engl J Med 317:287-94.
4. Obeid K M. 2019. Infections with DNA Viruses, Adenovirus, Polyomaviruses, and Parvovirus B19 in Hematopoietic Stem Cell Transplant Recipients and Patients with Hematologic Malignancies. Infect Dis Clin North Am 33:501-521.
5. Brown T, Anand A, Ritchie L D, Clewley J P, Reid T M. 1984. Intrauterine parvovirus infection associated with hydrops fetalis. Lancet 2:1033-4.
6. Guidance for Industry Nucleic Acid Testing (NAT) to Reduce the Possible Risk of Human Parvovirus B19 Transmission by Plasma-Derived Products, U.S. Department of Health and Human Services Food and Drug Administration Center for Biologics Evaluation and Research, July 2009.
7. Molenaar-de Backer M W, Russcher A, Kroes A C, Koppelman M H, Lanfermeijer M, Zaaijer H L. 2016. Detection of parvovirus B19 DNA in blood: Viruses or DNA remnants? J Clin Virol 84:19-23.
8. Eid A J, Ardura M I, Practice ASTIDCo. 2019. Human parvovirus B19 in solid organ transplantation: Guidelines from the American society of transplantation infectious diseases community of practice. Clin Transplant 33:e13535.
9. Watanabe K, Otabe K, Shimizu N, Komori K, Mizuno M, Katano H, Koga H, Sekiya I. 2018. High-sensitivity virus and mycoplasma screening test reveals high prevalence of parvovirus B19 infection in human synovial tissues and bone marrow. Stem Cell Res Ther 9:80.
10. Bua G, Manaresi E, Bonvicini F, Gallinella G. 2016. Parvovirus B19 Replication and Expression in Differentiating Erythroid Progenitor Cells. PLoS One 11:e0148547.
11. WO2007/140011
12. Kitamura T, Tange T, Terasawa T, Chiba S, Kuwaki T, Miyagawa K, Piao Y F, Miyazono K, Urabe A, Takaku F. 1989. Establishment and characterization of a unique human cell line that proliferates dependently on GM-CSF, IL-3, or erythropoietin. J Cell Physiol 140:323-34.
13. Kitamura T, Tojo A, Kuwaki T, Chiba S, Miyazono K, Urabe A, Takaku F. 1989. Identification and analysis of human erythropoietin receptors on a factor-dependent cell line, TF-1. Blood 73:375-80.
14. Gallinella G, Manaresi E, Zuffi E, Venturoli S, Bonsi L, Bagnara G P, Musiani M, Zerbini M. 2000. Different patterns of restriction to B19 parvovirus replication in human blast cell lines. Virology 278:361-7.
15. Miyagawa E, Yoshida T, Takahashi H, Yamaguchi K, Nagano T, Kiriyama Y, Okochi K, Sato H. 1999. Infection of the erythroid cell line, KU812Ep6 with human parvovirus B19 and its application to titration of B19 infectivity. J Virol Methods 83:45-54.
16. Morita E, Tada K, Chisaka H, Asao H, Sato H, Yaegashi N, Sugamura K. 2001. Human parvovirus B19 induces cell cycle arrest at G(2) phase with accumulation of mitotic cyclins. J Virol 75:7555-63.
17. Komatsu N, Nakauchi H, Miwa A, Ishihara T, Eguchi M, Moroi M, Okada M, Sato Y, Wada H, Yawata Y, et al. 1991. Establishment and characterization of a human leukemic cell line with megakaryocytic features: dependency on granulocyte-macrophage colony-stimulating factor, interleukin 3, or erythropoietin for growth and survival. Cancer Res 51:341-8.
18. Manaresi E, Bua G, Bonvicini F, Gallinella G. 2015. A flow-FISH assay for the quantitative analysis of parvovirus B19 infected cells. J Virol Methods 223:50-4.
19. Miura et al., 1990
20. Norio Komatsu, Keita Kirito, Ritsuko Shimizu, Masae Kunitama, Minami Yamada, Mie Uchida, Masaaki Takatoku, Mituoki Eguchi, and Yasusada Miura. In Vitro Development of Erythroid and Megakaryocytic Cells From a UT-7 Subline, UT-7/GM. Vol: 89, Issue: 11, Page: 4021-4033, 1997.
21. Wong S, Zhi N, Filippone C, Keyvanfar K, Kajigaya S, Brown K E, Young N S. 2008. Ex vivo-generated CD36+ erythroid progenitors are highly permissive to human parvovirus B19 replication. J Virol 82:2470-6.
22. Wong S, Brown K E. 2006. Development of an improved method of detection of infectious parvovirus B19. J Clin Virol 35:407-13.
23. Ganaie et al., J Virol. 2018.

1. A human erythroid progenitor cell line, wherein at least 90% of the cells are CD36⁺CD44⁻CD71⁺; and wherein the cells:
do not express the gene encoding the receptor of Granulocyte-macrophage colony-stimulating factor (GM-CSF-R gene) or express GM-CSF-R gene at a lower level than the cells of human UT-7/Epo-S1 cell line; and
express the gene encoding the receptor of erythropoietin (Epo-R gene).

2. The cell line of claim 1, wherein Signal Transducer and Activator of Transcription 5 (STAT-5) is not phosphorylated or phosphorylated at a lower level than in human UT-7/Epo-S1 cell line, when stimulated with GM-CSF.

3. The cell line of claim 1, wherein Integrin-α5 (CD49e) is expressed at a higher level than in human UT-7/Epo-S1 cell line.

4. The cell line of claim 1, wherein the cells are strictly dependent on erythropoietin (Epo) for growth.

5. The cell line of claim 1, wherein the cell line is immortalized.

6. The cell line of claim 1, wherein the cell line is derived directly or indirectly from a human megakaryoblastoid cell line.

7. The cell line of claim 1, wherein the cell line is UT7/Epo-STI, deposited under the provisions of Budapest treaty, at the Collection Nationale de Cultures de Microorganismes (CNCM, having the address: CNCM, Institut Pasteur, 25 rue du Docteur Roux, F-75724 Paris Cedex 15), on 5 Oct. 2020, under the deposit number CNCM I-5599.

8. The cell line of claim 1, wherein the cell line expresses at least one gene encoding a cell cycle indicator.

9. The cell line of claim 8, wherein the cell cycle phases are detected using the FUCCI system.

10. The cell line of claim 8, wherein the cells do not express the gene encoding GM-CSF-R.

11. The cell line of claim 8, wherein the cell line is UT7/Epo-STI-derived clone E2, deposited under the provisions of Budapest treaty, at the Collection Nationale de Cultures de Microorganismes (CNCM, having the address: CNCM, Institut Pasteur, 25 rue du Docteur Roux, F-75724 Paris Cedex 15), on 5 Oct. 2020, under the deposit number CNCM I-5600.

12. The cell line of claim 1, having a high permissivity/sensitivity for human parvovirus B19 infection.

13. Method for producing parvovirus B19 in vitro, using the cell line according to claim 1.

14. The method of claim 13, wherein the parvovirus B19 is a native parvovirus B19 or a recombinant parvovirus B19.

15. Method for detecting infectious parvovirus B19 in vitro in a biological sample; or for diagnosing in vitro a B19 infection in a subject of interest, from a biological sample of said subject; or for screening compounds/active agents in vitro for parvovirus B19 antiviral activity/effect; using the cell line according to claim 1.

16. The method of claim 15, for quantifying infectious parvovirus B19 in vitro, in particular for evaluating the efficiency of a viral reduction process on infectious parvovirus B19 and/or for diagnosing a B19 infection in a subject of interest.

17. (canceled)

18. (canceled)

19. The cell line of claim 6, wherein the cell line is derived directly or indirectly from human UT-7 cell line.

20. The cell line of claim 8, wherein the cell cycle indicator is a fluorescent cell cycle Indicator.

21. The cell line of claim 20, wherein the cell cycle indicator is Fluorescent Ubiquitination Cell Cycle Indicator (FUCCI).

22. The cell line of claim 12, wherein the permissivity/sensitivity of the cell line for human parvovirus B19 infection is at least 5 times higher compared to human UT-7/Epo-S1 cell line when detected/quantified at the RNA level.
